(19) United States
(12) Patent Application Publication
(10) Pub. No.: US 2025/0152324 A1
(43) Pub. Date: May 15, 2025

(54) MULTILAYER IMPLANTABLE CELL ENCAPSULATION DEVICES AND METHODS THEREOF

(71) Applicant: ViaCyte, Inc., San Diego, CA (US)

(72) Inventors: Mario Zamarripa, San Diego, CA (US); Craig McGreevy, San Diego, CA (US); Jonathan Valdez, Chula Vista, CA (US); Christopher Folk, San Diego, CA (US); Michael Scott, North York (CA); Vahid Hamzeinejad, San Diego, CA (US); Qiang Zhang, Annandale, NJ (US)

(73) Assignee: ViaCyte, Inc., San Diego, CA (US)

(21) Appl. No.: 18/838,343

(22) PCT Filed: Feb. 24, 2023
(86) PCT No.: PCT/US2023/013878

§ 371 Date: Aug. 14, 2024

Related U.S. Application Data
(60) Provisional application No. 63/313,888, filed on Feb. 25, 2022.

(51) Int. Cl. A61F 2/02

(52) U.S. Cl. CPC A61F 2/022

(57) ABSTRACT

Described is a multilayer cell encapsulation device and methods of assembly and uses thereof. In some examples, a multilayer cell encapsulation device can include a non-non-woven fabric (NWF) layer defining at least one chamber configured to receive cells therein, and a non-woven fabric (NWF) layer arranged external to the non-NWF layer, where the NWF layer and the non-NWF layer are suction welded together. At least a portion of a perimeter of at least the non-NWF layer which forms an outer weld of the device comprises a plurality of spaced apart flow holes extending along the perimeter.

### CROSS-REFERENCE TO RELATED APPLICATIONS

This application claims the benefit of U.S. Provisional Application No. 63/313,888, filed Feb. 25, 2022, which is incorporated by reference herein in its entirety.

### FIELD OF DISCLOSURE

The present disclosure relates generally to a cellular therapy and methods for making and using an implantable multilayer device capable of being loaded with a therapeutic agent or biologically active agent, such as cells. Such an implantable multilayer device can then be transplanted into a mammal, such as a human, and used for the treatment of a human disease, for example, diabetes mellitus.

### BACKGROUND OF DISCLOSURE

A therapeutic agent encapsulation device can comprise multiple layers with the center two layers of the device comprising a non-woven fabric (NWF) layer attached to a semi-permeable membrane. The semi-permeable membrane contains the therapeutic agent, for example, cells. The purpose of the membrane is to allow nutrient transport to the contained cell population, as well as providing the cell population with protection from a host's immune system. The remaining layers can include one or more mesh layers and film layers. The film layers are intercalated in between the mesh layer and the NWF layer/membrane layer, and are melted during the assembly process in order to create a device perimeter weld and interior weld necessary to achieve encapsulation of the cells. The NWF layer attached to the membrane enhances in-vivo integration and promotes host vascularization. The implanted devices can remain in the subject for several months to several years, thus, the integrity of the device must be preserved.

Currently, ultrasonic welding or heat staking are common assembly methods used to melt the film layers in order to achieve bonding of the device perimeter weld and internal welds. During the welding process, ultrasonic welding energy is transmitted through the multiple device layers using a sonotrode that applies force from the top of a stack of multiple layers. During the heat staking process, heat and pressure are applied in a similar manner using a tool called an iron. Both ultrasonic and heat energy attenuate as energy is transmitted through the multiple layers; the energy is particularly attenuated by the membrane component, which has a high melting point as compared to the film layers. Thus, using either method, it is challenging to apply the appropriate combination of parameters (e.g., heat or energy) to achieve adequate bonding of the layers to create a strong weld, without damaging layers that have a lower melting point than the rest of the layers.

As a result of uneven welds in the device, the integrity of the device can be reduced and may become degraded and/or separate along the perimeter (or external weld) of the device or along an internal weld, due to the internal pressure of the growing cells, or external shear stress from the surrounding host tissue. The weakened welds can result in broken or burst welds or layers of the device “peeling” back or delaminating.

Therefore, there is a need for device membrane design features in the weld area, that allow for better heat transfer or ultrasonic welding energy transfer through multiple layers, in order to enhance overall weld strength of multilayered devices containing therapeutic agents. There is also a need for improved methods of assembling multilayer devices, that result in enhanced overall weld strength, thus preventing weld failures (e.g., broken or burst welds) or delamination of layers of the device which can lead to cell egress from the device or host tissue ingrowth into the device. The mesh has a lower melting point than the membrane. Therefore, there is a need for protecting the additional layers of the device, such as the mesh component, by minimizing the amount of energy applied during the manufacturing process of the multilayered device.

### SUMMARY

The following membrane features can added to enable stronger bonding: 1) flow holes are laser cut in portions of the membrane perimeter that will form the exterior (outer) weld, 2) an interior middle slit is laser cut along the longest axis of the device (in between the device lumens), that will ultimately form an interior weld, and 3) a recessed outer edge, wherein the membrane is recessed along the device perimeter in relationship to the surrounding layers (e.g. mesh, film), and results in the formation of an “end cap”. Any one or all three of these features can be added to both the membrane and the NWF layer but can also be added only to the membrane. Any one or more of these features allows the intercalated film layers to come in direct contact with each other during the welding process, allowing for increased flow of melted film material through each layer, creating a stronger bond around the perimeter and in between the lumens of the device.

In addition, the shape of the device can also be cut during the laser tack welding process. Laser tack welding the NWF to the membrane results in increased stiffness of the laser tack welded materials, thus reducing the likelihood of damage to the membrane.

In the finished device, the NWF/membrane layer is “sandwiched” between two layers of film. The end cap is made by recessing the NWF/membrane layer around the perimeter, so that the two film layers that sandwich the NWF/membrane melt into each other (film-on-film contact) when the NWF/membrane layer is combined with the remaining layers and welded together.

Any one of the three membrane features, significantly increase the weld strength of a cell encapsulation device. During the welding process (e.g., heat staking or ultrasonic welding, which can also be referred to as ultrasonic vibration), the film melts through the flow holes located along the perimeter of the NWF/membrane and also melts the film into the interior middle slit of the NWF/membrane, creating strong welds throughout the cell encapsulation device. The exterior welds are further strengthened by recessing the NWF/membrane layer to allow film-on-film melting during welding. The film (e.g., Bionate) has a melting temperature that is significantly lower than the other materials in the assembly such as the membrane).

Disclosed herein are multi-layered cell encapsulation devices, comprising: a non-woven fabric (NWF) layer and a non-NWF layer, wherein the NWF layer and the non-NWF layer are welded (e.g., suction welded) together and comprise device perimeters comprising flow-holes. In one embodiment, the non-NWF layer is a semi-permeable membrane. In another embodiment, the semi-permeable membrane is made of polytetrafluoroethylene (PTFE) or expanded polytetrafluoroethylene (ePTFE). In another embodiment, the non-NWF layer is a cell-excluding membrane. In another embodiment, the non-NWF layer is a vascularizing membrane. In one embodiment, the non-NWF layer is a mesh. In another embodiment, the non-NWF layer is perforated. In one embodiment, the NWF layer and the non-NWF layer are perforated. In another embodiment, the suction welding involves laser tack welding. In one embodiment, the flow holes have a length or a diameter of about 0.001 mm to about 20 mm. In another embodiment, the device has a longest axis and the flow holes are located on the top and bottom perimeters of the device along the longest axis. In one embodiment, cells are loaded into the device. In another embodiment, the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells. In one embodiment, the cells are aggregates. In another embodiment, the NWF layer and the non-NWF layer further comprise a recessed perimeter when placed in between a first and second film layer.

Disclosed herein is a multi-layered cell encapsulation device, comprising: a NWF layer and a non-NWF layer, wherein the NWF layer and non-NWF layer are welded (e.g., suction welded) together and comprise device perimeters comprising flow-holes; and a first film layer welded to one side of the suction welded NWF layer and non-NWF layer and a second film layer welded to the opposite side of the suction welded NWF layer and non-NWF layer.

Also, disclosed herein is a multi-layered cell encapsulation device, comprising: a NWF layer and a non-NWF layer, wherein the NWF layer and the non-NWF layer are welded (e.g., suction welded) together and are placed between a first and second film layer, wherein the perimeter of the NWF layer and non-NWF layer are recessed in relationship to the first and second film layers.

Also disclosed herein is a multi-layered cell encapsulation device, comprising pancreatic lineage cells and a NWF layer and a non-NWF layer, wherein the NWF layer and the non-NWF layer are welded (e.g., suction welded) together and comprise device perimeters comprising flow-holes.

Disclosed herein are methods for welding and cutting a multi-layered cell encapsulation device, comprising obtaining a NWF layer and a non-NWF layer, and welding (e.g., suction welding) the NWF layer and non-NWF layer while cutting a device perimeter and flow holes in the perimeter of the device. In one embodiment, the suction welding and cutting is laser tack welding. In another embodiment, the non-NWF layer is a semi-permeable membrane. In one embodiment, the semi-permeable membrane is made of polytetrafluoroethylene (PTFE) or expanded polytetrafluoroethylene (ePTFE). In another embodiment, the non-NWF layer is a cell-excluding membrane. In one embodiment, the non-NWF layer is vascularizing membrane. In another embodiment, the non-NWF layer is a mesh. In one embodiment, the non-NWF layer is perforated. In another embodiment, the NWF layer and the non-NWF layer are perforated. In one embodiment, the suction welding is laser tack welding. In another embodiment, the flow holes have a length or a diameter of about 0.001 mm to about 20 mm. In one embodiment, the device has a longest axis and the flow holes are located on the top and bottom perimeters of the device along the longest axis.

### BRIEF DESCRIPTION OF THE DRAWINGS

These and other features, aspects, and advantages of the present disclosure will become better understood with regard to the following description, appended claims and accompanying figures where:

FIG. 1 is an exploded view of a cell encapsulation device comprising multiple layers stacked together, the multiple layers including (from the outside to the middle of the device) oppositely arranged non-woven fabric rings, film rings, membrane layers including non-woven fabric welded thereto, and additional film rings.

FIG. 2 is an exploded view of a cell encapsulation device comprising multiple layers stacked together, the multiple layers including (from the outside to the middle of the device) oppositely arranged non-woven fabric layers, film rings, mesh layers, and membrane layers including non-woven fabric welded thereto, and additional film rings.

FIG. 3 is an exploded view of a cell encapsulation device comprising multiple layers stacked together, the multiple layers including (from the outside to the middle of the device) oppositely arranged mesh layers, film rings, membrane layers including non-woven fabric welded thereto, and additional film rings.

FIG. 4 is an exploded view of a cell encapsulation device comprising multiple layers stacked together, the multiple layers including six layers of film and two membrane layers forming therapeutic agent-receiving chambers of the device, where the membrane layers include flow holes, recessed outer edges forming an end cap feature, and an internal flow slit which are configured to increase a strength of the internal and outer welds of the device.

FIG. 5 is a plan view of a membrane layer of the device of FIG. 4, with a non-woven fabric layer welded thereto, which illustrates the flow holes and internal flow slit in the non-woven fabric/membrane layer.

FIG. 6A is a detail view of a portion of the non-woven fabric/membrane layer of FIG. 5 showing the flow holes arranged in a linear array along a longitudinally extending edge of the layer.

FIG. 6B is a detail view of a portion of a non-woven fabric/membrane layer that could be used in lieu of the non-woven fabric/membrane layer in the device of FIG. 4, where the flow holes have an alternative arrangement of two staggered linear arrays of flow holes.

FIG. 6C is a detail view of a portion of a non-woven fabric/membrane layer that could be used in lieu of the non-woven fabric/membrane layer in the device of FIG. 4, where the layer includes a linear arrange of flow slits instead of flow holes.

FIG. 6D is a detail view of a portion of a non-woven fabric/membrane layer that could be used in lieu of the non-woven fabric/membrane layer in the device of FIG. 4, where the layer includes a single longitudinally extending flow slit instead of a plurality of flow holes.

FIG. 7 is a plan view of the welded together non-woven fabric/membrane layer of FIGS. 4 and 5 with an outline of the underlying/overlying film rings provided for reference, thereby showing the recessed outer edge and the chambers of the device.

FIG. 8 is a cross-sectional view of the device of FIG. 4 showing the stacking of device layers and a recessed edge of the non-woven fabric/membrane layers relative to the other device layers.

FIG. 9 is a plan view of an exemplary non-woven fabric/membrane layer that could be used in the device of FIG. 4, where the non-woven fabric/membrane layer include a plurality of spaced apart internal slits in a region that forms the interior weld of the device.

FIG. 10 is a plan view of a portion of a cell encapsulation device comprising three chambers and a membrane layer comprising flow holes along its outer long edges, two rows of spaced apart internal slits, and an alternate arrangement of perforations.

FIG. 11 is a plan view of a portion of a cell encapsulation device comprising three chambers and a membrane layer comprising flow holes along its outer long edges, two rows of spaced apart internal slits, and no perforations.

FIG. 12 is a plan view of a portion of a cell encapsulation device that comprises two rows of staggered flow slits along the outer, long edges of the membrane layer.

FIG. 13 is a detail view of a portion of one of the outer, long edges of the membrane layer of the device of FIG. 12.

FIG. 14 is a plan view of the non-woven fabric/membrane layer of the device of FIG. 12.

FIG. 15 is an exploded view of one half of a cell encapsulation device comprising multiple layers stacked together, the multiple layers of the one half including three layers of film and a membrane layer including a recessed outer edge forming an end cap feature and an internal flow slit.

FIG. 16 is a plan view of the device of FIG. 15 which shows the recessed outer edge forming the end cap feature of the device.

FIG. 17A is an assembled view of the device of FIG. 15 showing the two chambers of the device.

FIG. 17B is a semi-assembled view of the device of FIGS. 15-17A with the device layers of each half of the device stacked and assembled together, but the two assembled halves shown separated from one another with a device port disposed therebetween.

FIG. 18 is an exploded view of a cell encapsulation device comprising multiple layers stacked together, the multiple layers including four layers of film and two membrane layers forming therapeutic agent-receiving chambers of the device, where the membrane layers do not include flow holes, a recessed outer edge forming an end cap, or an internal flow slit.

FIG. 19A is a plan view of a non-woven fabric layer attached to one of the membrane layers of the device of FIG. 18 with an outline of the film rings shown for the purpose of illustrating the chambers and outer edges of the layers of the device.

FIG. 19B is a plan view of one of the non-woven fabric/membrane layers of the device of FIG. 4 with an outline of the film rings shown for the purpose of illustrating the chambers and outer edges of the layers of the device, and to show the presence of the recessed outer edge, flow holes, and internal slit relative to the non-woven fabric/membrane layer of FIG. 19A.

FIG. 20 is a plan view of an example of the device shown in FIG. 18, but with different weld widths for the internal and outer welds.

FIG. 21A is a plan view of an example of the device shown in FIG. 18, but with different weld widths for the internal and outer welds.

FIG. 21B is a plan view of an example of the device shown in FIG. 4, but with an outer weld width that is larger than that of the device of FIG. 21A in order to accommodate the end cap in the device of FIG. 21B.

FIG. 22 is a perspective view of an exemplary device, apparatus, or holder for holding the layers of a cell encapsulation device during ultrasonic welding or heat staking of all the device layers together to form the device welds.

FIG. 23 is a bar graph of weld peel strength for a Test Device 2 compared to a Test Device 1, where Test Device 2 was made in accordance with the device of FIG. 18 and Test Device 1 was made in accordance with the device of FIG. 15.

FIG. 24 is a bar graph of burst pressure for Test Device 2 compared to Test Device 1.

FIG. 25 is a bar graph illustrating mean peel strength of the end cap feature of Test Device 1.

FIG. 26 is a bar graph illustrating weld peel strength of Test Device 2 for several different weld locations of the device.

FIG. 27 is a graph of weld peel data (or weld peel strength) of Test Device 2 articles compared to Test Device 3 articles.

FIG. 28 is a graph of burst pressure (or value) for Test Device 2 articles compared to Test Device 3 articles.

FIG. 29 is a graph of weld peel data (or weld peel strength) of Test Device 1 articles compared to Test Device 3 articles.

FIG. 30 is a graph of burst pressure (or value) for Test Device 1 articles compared to Test Device 3 articles.

FIG. 31 is a graph 960 of weld peel data (or weld peel strength) for a membrane comprising a single flow slit (such as that shown in FIG. 6D) compared to a membrane having flow holes (such as that shown in FIG. 7).

FIGS. 32A and 32C are SEM images of cross-sections of Test Device 3 articles, the SEM images illustrating good mesh layer/film ring/NWF-membrane layer intercalation after ultrasonic welding of the device layers to form the device.

FIG. 32B is a SEM image of a cross-section of a Test Device that is similar to Test Device 2 and illustrates a poor mesh layer/film ring/NWF-membrane intercalation after ultrasonic welding of the device layers to form the device.

FIG. 33 is a SEM image of a portion of a Test Device 2, which illustrates degradation of the device in the form of broken welds and mesh delamination.

FIGS. 34A and 34B are SEM images of detail portions of Test Device 2, which illustrate degradation of the device by mesh delamination.

FIG. 35A is an SEM image of a cross-section of Test Device 2, which does not include the end cap feature.

FIG. 35B is a SEM image of a cross-section of Test Device 1, which includes the end cap feature.

FIG. 36 is a plan view of Test Device 2 which illustrates delamination location measurements for the device.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

References in the specification to “one embodiment,” “an embodiment,” “an exemplary embodiment,” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. In addition, a range of “0.1% to 5%” should be interpreted to include not just 0.1% to 5%, but also the individual values (e.g., 1%, 2%, 3%, and 4%) and the sub-ranges (e.g., 0.1% to 0.5%, 1.1% to 2.2%, 3.3% to 4.4%) within the indicated range.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement).

Various publications including scientific journal articles, patent publications and patents are herein referred to and the disclosure of each of them is incorporated herein by reference in its entirety. For example, methods of making insulin-producing cells and the implantation of implantable devices with pancreatic progenitors derived from human pluripotent stem cells for the production of insulin-producing cells are disclosed in Applicants U.S. Pat. Nos. 7,534,608; 7,695,965; 7,993,920; 8,338,170; 8,278,106; and 8,425,928; all of which are incorporated by reference herein in their entireties. In the event of inconsistent usages between this document and those documents so incorporated by reference, the usage in the incorporated reference should be considered supplementary to that of this document; for irreconcilable inconsistencies, the usage in this document controls.

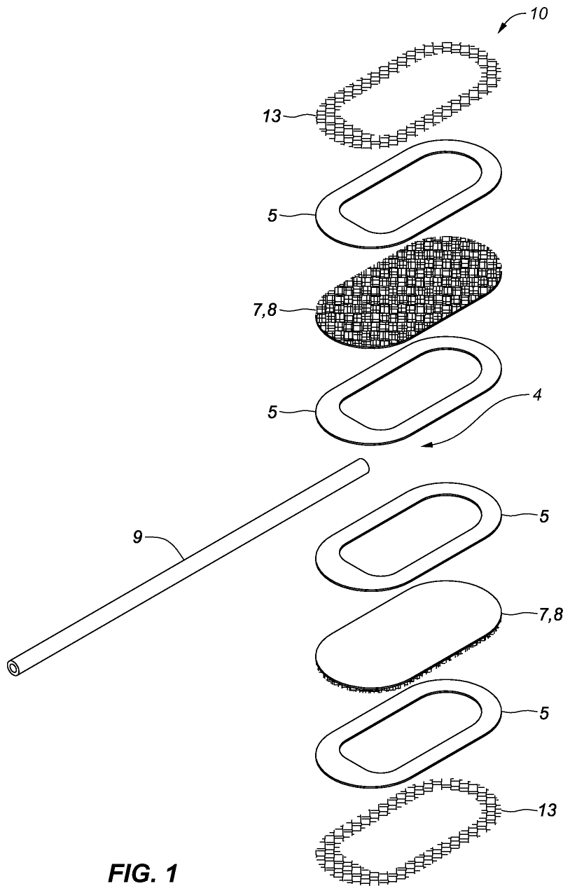

Further features and advantages of the disclosure, as well as the structure and operation of various embodiments of the disclosure, are described in detail below with reference to the accompanying FIGS. 1-36, wherein like reference numerals refer to like elements.

### INTRODUCTION

As used herein, the term “device” refers to any implantable device, macro-encapsulation device, cell-encapsulation device, or large capacity device, capable of being loaded with a therapeutic agent or biologically active agent.

In some examples, the therapeutic agents described herein can be encapsulated in a biological and/or non-biological mechanical device, where the encapsulated device separates and/or isolates the therapeutic agents from the host.

Methods of encapsulation are described in detail in U.S. Application 61/114,857, filed Nov. 14, 2008, titled ENCAPSULATION OF PANCREATIC PROGENITORS DERIVED FROM HES CELLS, and U.S. Application No. 61/121,086 filed Dec. 12, 2008, titled ENCAPSULATION OF PANCREATIC ENDODERM CELLS, all of which are incorporated by reference herein in their entireties.

In one example, cells derived from human embryonic stem cells are encapsulated using a bio-compatible polyethylene glycol (PEG). PEG-based encapsulation is described in more detail in U.S. Pat. No. 7,427,415, entitled IMPLANTATION OF ENCAPSULATED BIOLOGICAL MATERIALS FOR TREATING DISEASES; U.S. Pat. No. 6,911,227, entitled GELS FOR ENCAPSULATION OF BIOLOGICAL MATERIALS; and U.S. Pat. Nos. 6,911,227, 5,529,914, 5,801,033, 6,258,870, entitled GELS FOR ENCAPSULATION OF BIOLOGICAL MATERIALS, all of which are incorporated by reference herein in their entireties.

In some examples, one or more of the improvements disclosed herein (e.g., the flow holes, internal slit, or recessed outer edge forming device end caps) could be incorporated into a TheraCyte device (Irvine, Calif.). TheraCyte cell encapsulation devices are further described in U.S. Pat. Nos. 6,773,458; 6,156,305; 6,060,640; 5,964,804; 5,964,261; 5,882,354; 5,807,406; 5,800,529; 5,782,912; 5,741,330; 5,733,336; 5,713,888; 5,653,756; 5,593,440; 5,569,462; 5,549,675; 5,545,223; 5,453,278; 5,421,923; 5,344,454; 5,314,471; 5,324,518; 5,219,361; 5,100,392; and 5,011,494.

The devices described herein can be employed for treating pathologies requiring a continuous supply of biologically active substances to a host organism. Such devices, for example, can also be referred to as, bio artificial organs, which contain homogenous or heterogenous mixtures of biologically active agents (therapeutic agents) and/or cells, or cells producing one or more biologically active substances of interest. Ideally, the biologically active agents and/or cells are wholly encapsulated or enclosed in at least one internal space or encapsulation chambers, which are bounded by at least one or more semi-permeable membranes. Such a semi-permeable membrane should allow the encapsulated biologically active substance of interest to pass (e.g., insulin, glucagon, pancreatic polypeptide and the like), making the active substance available to the target cells outside the device and in the host organism's body. In one embodiment, the semi-permeable membrane allows nutrients naturally present in the host to pass through the membrane to provide essential nutrients to the encapsulated cells. At the same time, such a semi-permeable membrane prohibits or prevents the host's cells, particularly cells of the immune system, from passing through and into the device and harming the encapsulated cells in the device. For example, in the case of diabetes, this approach can allow glucose and oxygen to stimulate insulin producing cells to release insulin as required by the body in real time, while preventing immune system cells from recognizing and destroying the implanted cells. In one embodiment, the semi-permeable membrane prohibits the implanted cells from escaping encapsulation.

In some examples, the encapsulation device contains a pluripotent-derived cell, for example, a PDX-1 positive foregut endoderm cell, a PDX-1 positive pancreatic endoderm cell or progenitor cell, an endocrine or endocrine progenitor/precursor cell, such as an NGN3 positive endocrine progenitor/precursor cell, or a functional differentiated hormone secreting cell, such as an insulin, glucagon, somatostatin, ghrelin, or pancreatic polypeptide secreting cell, in a semipermeable membrane that prevents passage of the transplanted cell population, retaining them in the device, while at the same time permitting passage of certain secreted polypeptides, e.g., insulin, glucagon, somatostatin, ghrelin, pancreatic polypeptide and the like. Alternatively, the device has a plurality of membranes, including a vascularizing membrane.

An encapsulation device can be implanted into a mammal to treat a variety of diseases and disorders. In one embodiment, the device comprises a biocompatible, immuno-isolating device that is capable of wholly encapsulating a therapeutically biologically active agent and/or cells therein. For example, such devices can house therapeutically effective quantities of cells within a semipermeable membrane having a pore size such that oxygen and other molecules important to cell survival and function can move through the semi-permeable membrane, but the cells of the immune system cannot permeate or traverse through the pores. Similarly, such devices can contain therapeutically effective quantities of a biologically active agent, e.g., an angiogenic factor, a growth factor, a hormone and the like.

In some examples, the device contains a first membrane which is impermeable to cells (0.4 microns) but at the same does not restrict movement of oxygen and various nutrients in and out of the inner membrane, e.g., glucose from outside the inner membrane can permeate into the capsule containing the mature pancreatic hormone secreting cells, which in response to the glucose, can secrete insulin which then permeates out of the inner membrane. The device also contains an outer vascularizing membrane.

The disclosed devices may have certain characteristics which are desirable but are not limited to one or a combination of the following: i) comprised of a biocompatible material that functions under physiologic conditions, including pH and temperature; examples include, but are not limited to, anisotropic materials, polysulfone (PSF), nanofiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, as well as hydroxylpropyl methyl cellulose (HPMC) membranes; ii) releases no toxic compounds harming the biologically active agent and/or cells encapsulated inside the device; iii) promotes secretion or release of a biologically active agent or macromolecule across the device; iv) promotes rapid kinetics of macromolecule diffusion; v) promotes long-term stability of the encapsulated cells; vi) promotes vascularization; vii) comprised of membranes or housing structure that is chemically inert; viii) provides stable mechanical properties; ix) maintains structure/housing integrity (e.g., prevents unintended leakage of toxic or harmful agents and/or cells); x) is refillable and/or flushable; xi) is mechanically expandable; xii) contains no ports or at least one, two, three or more ports; xiii) provides a means for immuno-isolating the transplanted cells from the host tissue; xiv) is easy to fabricate and manufacture; and xv) can be sterilized.

The embodiments of the devices described herein are in not intended to be limited to certain device types, sizes, shapes, materials, configurations, designs, volume capacities, and/or materials used to make the encapsulation devices, so long as one or more of the above elements are achieved. For example, the device design can be in the shape of a tube or flattened tube or any other such shape which satisfies one of the above requirements for a device of the disclosure. One skilled in the art can modify the devices described herein without departing from the general embodiments.

A device of any size or shape reasonable can be further compartmentalized into having at least 1, at least 2, at least 3, at least 4, at least 5, at least 6, at least 7, at least 8, at least 9, at least 10, at least 11, at least 12, at least 13, at least 14, at least 15, at least 16, least 17, at least 18, at least 19, at least 20, at least 21, at least 22, at least 23, at least 24 or more chambers or compartments. One purpose for creating a plurality of compartments is that it increases the surface area for nutrient and oxygen exchange between the encapsulated cells and, for example, the interstitial space surrounding the device. In other embodiments, the device assemblies consist of one or two or more seals that further partition the lumen of the device, i.e., a partition seal. See, e.g., Applicant's U.S. Design applications 29/408,366, 29/408,368, 29/408,370 and 29/423,365. Such designs prohibit, reduce, or do not promote large cell aggregates or clusters or agglomerations such that cells packed in the center of the large clusters/agglomerations are denied, or receive less, nutrients and oxygen and therefore potentially do not survive. Devices containing a plurality of chambers or compartments therefore are better capable to disperse the cells throughout the chamber/compartment or chambers/compartments. In this way, there is more opportunity for each cell to receive nutrients and oxygen, thereby promoting cell survival and not cell death.

One example relates to a device or assembly consisting of substantially elliptical to rectangular shape cell chambers. These devices are further compartmentalized or reconfigured so that there is a weld or seam running through the center of the device, either sealing off each half of the device, thus forming two separate reservoirs, lumens, chambers, void spaces, containers or compartments; or the weld or seam creates an accordion-shaped chamber which is separated or divided in the middle due to the weld but such a weld in this instance does not completely seal off the chambers.

In some examples, the device is a perforated semi-permeable device comprising human pancreatic endocrine cells or PDX1-positive pancreatic endoderm cells within a semi-permeable membrane comprising a synthetic material, wherein the synthetic material is polysulfone (PSF), nano-fiber mats, polyimide, tetrafluoroethylene/polytetrafluoroethylene (PTFE), expanded polytetrafluoroethylene (ePTFE), polyacrylonitrile, polyethersulfone, acrylic resin, cellulose acetate, cellulose nitrate, polyamide, or hydroxylpropyl methyl cellulose (HPMC); a cell encapsulation chamber; and at least one seal that is within the cell encapsulation chamber, wherein the at least one seal within the cell encapsulation chamber does not increase the surface area of the cell encapsulation chamber relative to the absence of the at least one seal.

Also provided is an implantable device, which is immobilized at an implantation site to maintain the encapsulated cell and/or biological active agent at the implantation site and permit diffusion of, for example, an expressed and secreted therapeutic polypeptide from the implantation site.

CELL ENCAPSULATION DEVICE CONFIGURATIONS: Cell encapsulation devices include various layers each of which serves a function or multiple functions. In some examples, the cell encapsulation device includes both a cell-excluding membrane (which can also be referred to as a “membrane” herein) and a non-woven fabric.

CELL EXCLUDING MEMBRANE: A cell excluding membrane inhibits cellular components of the immune system such as T-cells and the like from entering the device. This layer also serves to keep the therapeutic cells from exiting the device. This layer allows the encapsulated biologically active substance of interest to pass (e.g., insulin, glucagon, pancreatic polypeptide and the like), making the active substance available to the target cells outside the cell encapsulation device and in the patient's body. This layer ideally allows nutrients naturally present in the host to pass through the membrane to provide essential nutrients to the encapsulated cells. Cell-excluding membranes have been described in the art including those patents previously described above by Baxter including, U.S. Pat. Nos. 6,773,458; 6,520,997; 6,156,305; 6,060,640; 5,964,804; 5,964,261; 5,882,354; 5,807,406; 5,800,529; 5,782,912; 5,741,330; 5,733,336; 5,713,888; 5,653,756; 5,593,440; 5,569,462; 5,549,675; 5,545,223; 5,453,278; 5,421,923; 5,344,454; 5,314,471; 5,324,518; 5,219,361; 5,100,392; and 5,011,494 which are all incorporated herein by reference in their entirety. In some embodiments this layer is perforated.

FILM: In some examples, the cell encapsulation device includes a film layer, film ring, or film weld. The film is a binding or adhesive layer that is only present in the weld that helps adhere or bond at least two or more layers together. In some examples the film is only on the interior face (chamber facing) of the fabric (see below), to eliminate the smooth surface that it creates if it were on the outer face (host facing), which inhibits anchoring. In some examples, the film is on the interior face (chamber facing) of the cell-excluding membrane. The film is not part of the chamber; it is located in the weld. Upon welding, the film melts through the layers of the device creating external welds. Upon welding, the film melts through the layers of the device creating internal welds. The film has a lower melting point than the other layers of a device.

MESH: A woven mesh provides structural rigidity to each device and protects the cell excluding membrane by serving as a protective exoskeleton. In some examples, a woven mesh is not included in the device. To address the loss of rigidity resulting from not including woven mesh from the device design, a double layer of non-woven fabric and/or a ring of non-woven fabric on the outside of the device may be used (e.g., as shown in FIG. 1).

NON-WOVEN FABRIC: A cell encapsulation device that becomes well-integrated into a host after implantation is provided. To this end, reducing, inhibiting or decreasing biofouling at the device-host interface is critical for device integration. In some examples, to improve the interface between the host's tissue and the implantable device, a non-woven fabric (NWF) (or NWF layer) is provided, for example, a non-woven polyester fabric (NWPF), external to a cell excluding membrane to improve graft vascularization. In addition, perforations can occur in either the NWF and cell-excluding membrane, or just the cell-excluding membrane, to improve graft vascularization. Such devices are described in PCT/US2016/061442 entitled PDX1 PANCREATIC ENDODERM CELLS IN CELL DELIVERY DEVICES AND METHODS THEREOF, filed Nov. 10, 2016, which is incorporated by reference herein in its entirety.

The term “non-woven fabric” or equivalents thereof, includes, but is not limited to, bonded fabrics, formed fabrics, or engineered fabrics, that are manufactured by processes other than, weaving or knitting. There are numerous types of non-woven fabrics, varying in density of fibers, amount of fibers, porosity and/or thickness of the non-woven fabric sheet. In some examples, the non-woven fabric comprises fibers or filaments that are trilobal in cross section. In some examples, the non-woven fabric is a porous, textile-like material, usually in flat sheet form, composed primarily or entirely of fibers, such as staple fibers assembled in a web, sheet or batt. The structure of the non-woven fabric is based on the arrangement of, for example, staple fibers that are typically arranged more or less randomly. Non-woven fabrics can be created by a variety of techniques known in the textile industry. Various methods may create carded, wet laid, melt blown, spun bonded, or air laid nonwovens. Exemplary methods and substrates are described in U.S. Application Publication No. 2010/0151575, which is incorporated by reference herein in its entirety. In one embodiment the non-woven fabric is polytetrafluoroethylene (PTFE). In one embodiment the non-woven fabric is a spunbound polyester.

The density of the non-woven fabric varies depending upon the processing conditions. In one example, the non-woven fabric is a spunbound polyester with a basic weight from about 0.40 to about 1.00 (oz/yd²), a nominal thickness of about 127 to about 228 μm, and a fiber diameter of about 0.5 to about 26 μm. In one example, the filament cross section is trilobal. In some examples, the non-woven fabrics are biocompatible and/or bioabsorbable.

In one example, a non-woven fabric is used to explore whether, along with providing structural integrity, it can increase vascularization and decrease or inhibit biofouling. In one example, the non-woven fabric provides protection to the cell-excluding membrane from direct contact with the woven mesh, and additional material for device anchoring to the host or device integration.

A NON-NWF LAYER: A non-NWF layer can be, for example, a membrane (e.g., a cell excluding membrane, semi-permeable membrane, or a vascularizing membrane), a film layer, a woven layer (e.g., mesh), or any other layer that is not defined herein as a NWF. Wherever the term “membrane” is used herein, the term “non-NWF” can also be used interchangeably. A non-NWF layer can be, for example, a layer of the device that is fragile and that could be damaged during manufacturing of the device.

Historically, cell encapsulation devices have a simple configuration, including just the cell-excluding membrane for therapeutic cell containment, film for welding, and the woven mesh.

### Examples of the Disclosed Technology

As introduced above, a therapeutic agent or cell encapsulation device (which is also referred to herein as a “device”) can comprise multiple layers with more central layers of the device comprising a non-woven fabric (NWF) layer attached to a semi-permeable membrane (or membrane layer). In some examples, two opposing semi-permeable membrane layers (arranged on opposite sides or halves of the device) can define one or more chambers or lumens therebetween that are configured to contain the therapeutic agent, for example, cells. The remaining layers of the device can include one or more mesh layers and film layers. The film layers are intercalated in between the mesh layer and the NWF layer/membrane layer, and are melted during the assembly process in order to create a device perimeter weld and interior weld necessary to achieve encapsulation of the cells.

In some examples, a cell encapsulation device, such as those described herein, could alternatively be referred to as a cell delivery device in so far as it is used to implant or position cells within a body of a host.

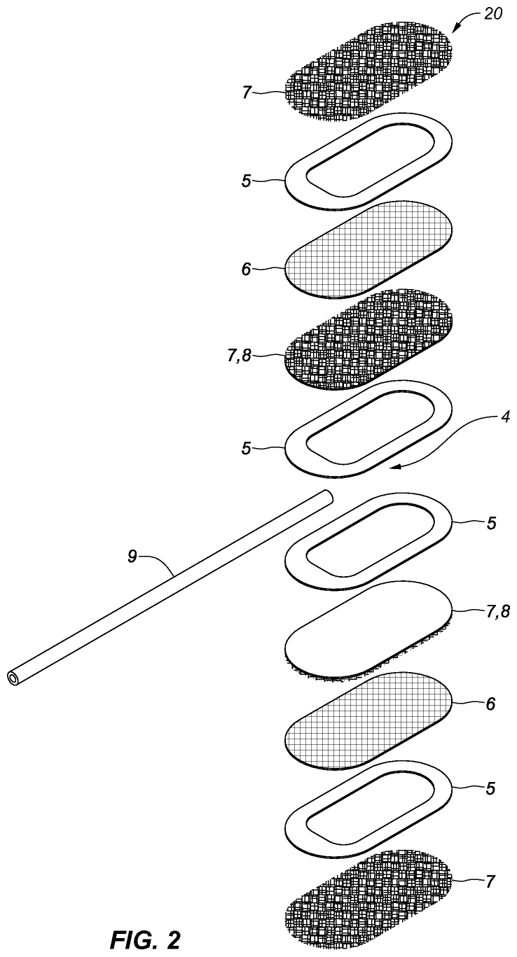

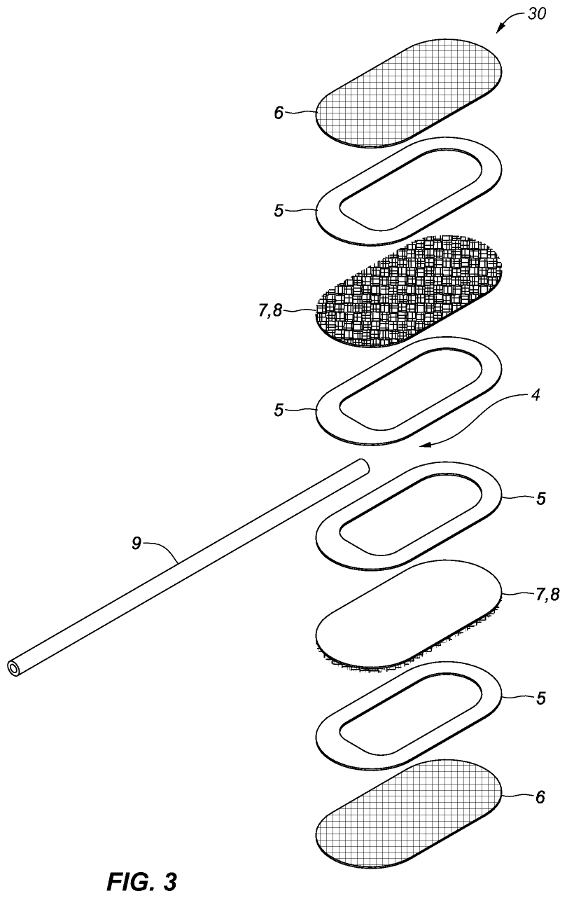

FIGS. 1-3 are exploded views of several examples of multi-layer cell encapsulation devices comprising a NWF layer 7 coupled (e.g., tack welded) to a membrane 8 and various additional layers that can include one or more of a film ring 5, mesh layer 6, additional NWF layer 7, and/or NWF ring 13. Each of the devices 10 (FIG. 1), 20 (FIG. 2), and 30 (FIG. 3) can be manufactured as a “sandwich” that is assembled as a stack of materials or layers (in various orders) and sealed.

FIG. 1 is an exploded view of an exemplary cell encapsulation device 10 that comprises (from the exterior to the interior or middle) a non-woven fabric (NWF) ring 13, a first film ring 5, a membrane layer 8 (which can also be referred to as a cell-excluding membrane 8) attached or fixed to a NWF layer 7. The NWF layer 7 attached to the membrane layer 8 enhances in-vivo integration and promotes host vascularization.

The NWF layer 7 can be heat laminated, welded, bonded, or laser tack welded to the membrane layer 8. A lay-up of one or more NWF layers and a membrane layer welded or otherwise bonded together can be referred to as a combination or composite NWF/membrane layer. In some examples, the NWF layer 7 can comprise two layers of NWF that are laminated, heat laminated, welded, bonded, or laser tack welded to the membrane layer 8. The NWF layer 7 faces out toward the host while the cell-excluding membrane layer 8 faces in toward a chamber 4 (or lumen) configured to receive implanted cells (or another therapeutic agent). The cell encapsulation device 10 further comprises a second film ring 5 at the periphery or weld. This pattern is then repeated for the other device wall of the device 10. The opposing side of the device 10 (from the exterior to the interior) includes a NWF ring 13, a third film ring 5, a membrane layer 8 wherein two layers of NWF, forming a NWF layer 7, are coupled, such as laminated, heat laminated, welded, bonded, or laser tack welded to the cell-excluding membrane 8. The NWF layer 7 faces out toward the host while the cell-excluding membrane layer 8 faces in toward the chamber 4 (and the implanted cells contained therein). A fourth film ring 5 can be included at the periphery or weld of the device 10. In this way, the two sides or walls of the device, with the chamber 4 contained therebetween, are mirror images of each other. The device 10 further comprises a port 9 sandwiched between the two walls of the device 10 (e.g., between the two central-most film rings 5) and extending from an exterior of the device 10 to an interior of the chamber 4 (or lumen). As such, the port 9 is configured for loading cells (or alternate therapeutic agent) into the chamber 4. In this example, the mesh layer is not included. In some examples, rather than a NWF ring 13, the entire surface of the device 10 is covered in NWF 7 (or a NWF layer 7) (such as shown in FIG. 2, as described below).

FIG. 2 is an exploded view of an exemplary cell encapsulation device 20 that comprises (from the exterior to the interior or middle) a NWF layer 7, a first film ring 5, a mesh layer 6, and a cell-excluding membrane layer 8 attached or fixed to a NWF layer 7.

The NWF layer 7 can be laminated, heat laminated, welded, bonded, or laser tack welded to the membrane layer 8. The NWF layer 7 faces out toward the host while the cell-excluding membrane layer 8 faces in toward a chamber 4 (or lumen) configured to receive implanted cells (or another therapeutic agent). The cell encapsulation device 20 further comprises a second film ring 5 (which forms the periphery or outer weld of the device with the peripheries of the other device layers).

This pattern is then repeated for the other device wall (or side) of the device 20. The opposing side of the device 20 (from the exterior to the interior) includes a NWF layer 7, a third film ring 5, a mesh layer 6, a cell-excluding membrane layer 8, wherein the NWF layer 7 is heat laminated, welded, bonded, or laser tack welded to the membrane layer 8, and a fourth film ring 5 at the periphery of the outer weld.

In this way, the two sides or walls of the device, with the chamber 4 contained therebetween, are mirror images of each other. The device 20 further comprises a port 9 sandwiched between the two wall of the device 20 (e.g., between the two central-most film rings 5) and extending from an exterior of the device 20 to an interior of the chamber 4 (or lumen). As such, the port 9 is configured for loading cells (or alternate therapeutic agent) into the chamber 4.

FIG. 3 is an exploded view of an exemplary cell encapsulation device 30 that comprises (from the exterior to the interior or middle) a mesh layer 6, a first film ring 5, a cell-excluding membrane layer 8 with a NWF layer 7 heat laminated, welded, bonded, or laser tack welded to the membrane 8, and a second film ring 5 at the periphery of the weld.

This pattern is then repeated for the other device wall (or side) of the device 30. The opposing side of the device 30 (from the exterior to the interior) includes a mesh layer 6, a third film ring 5, a cell-excluding membrane layer 8 with a NWF layer 7 heat laminated, welded, bonded, or laser tack welded to the membrane layer 8, and a fourth film ring 5 at the periphery of the outer weld.

In this way, the two sides or walls of the device, with the chamber 4 contained therebetween, are mirror images of each other. The device 30 further comprises a port 9 sandwiched between the two wall of the device 30 (e.g., between the two central-most film rings 5) and extending from an exterior of the device 30 to an interior of the chamber 4 (or lumen). As such, the port 9 is configured for loading cells (or alternate therapeutic agent) into the chamber 4.

The implanted devices described herein can remain in the subject for several months to several years, thus, the integrity of the device must be preserved. As introduced above, ultrasonic welding or heat staking are common assembly methods used to melt the film layers in order to achieve bonding of the device perimeter weld and internal welds. During the welding or heat staking process, ultrasonic welding energy or heat and pressure is transmitted through the multiple device layers from the top of a stack of the multiple device layers. Both ultrasonic and heat energy attenuate as energy is transmitted through the multiple layers, and the energy is particularly attenuated by the membrane layer, which has a high melting point as compared to the film layers. Thus, it is challenging to apply the appropriate combination of parameters (e.g., heat or energy) to achieve adequate bonding of the layers to create a strong weld, without degrading layers that have a lower melting point than the rest of the layers. In some examples, this can result in uneven welds in the device, which can reduce the integrity and longevity of the device.

Therefore, there is a need for device membrane design features in the weld area(s), that allow for better heat transfer or ultrasonic welding energy transfer through multiple layers, in order to enhance overall weld strength of multilayered devices containing therapeutic agents. There is also a need for improved methods of assembling multilayer devices, that result in enhanced overall weld strength and a reduction in the amount of energy applied to the device layers during the manufacturing process.

In some examples, the membrane layer or the combined NWF layer and membrane layer of a cell encapsulation device can include one or more features than enable stronger bonding between the multiple layers of the cell encapsulation device, thereby increasing the strength of the welds of the device and increasing an integrity and longevity of the device.

For example, the membrane layer or the combined NWF layer and membrane layer can include one or more of flow holes (or apertures or slits) that are cut through (e.g., laser cut during laser tack welding) the membrane layer or NWF/membrane layer along its perimeter which will form the exterior (outer) weld, an interior middle slit (or slits) (also referred to herein as an “internal slit” or “internal slits”) that is cut (e.g., laser cut) through the membrane layer or NWF/membrane layer along a portion of its interior (in between the chambers or lumens of the device) which will form an interior weld of the device, and/or a recessed outer edge where the membrane layer or NWF/membrane layer is recessed (inward toward its interior or central longitudinal axis) along its perimeter relative to the surrounding layers (e.g., the mesh, film, and/or the like) (and which results in the formation of device end caps). Any one of or all three of these features can be added to both the membrane layer and the NWF layer or added only to the membrane layer (and not the NWF layer attached to the membrane layer).

Any one or more of these features (the flow holes, internal slit, or recessed outer edge) allows the intercalated film layers of the device to come in direct contact with each other during the welding process, thereby allowing for increased flow of melted film material through each layer and creating a stronger bond around the perimeter of the device and in between the chambers or lumens of the device.

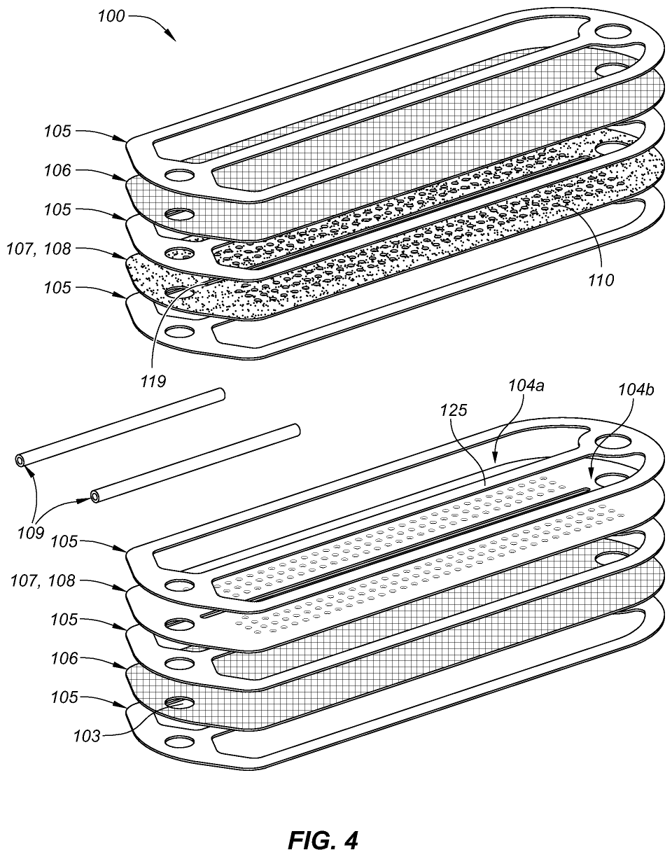

FIGS. 4-8 show an exemplary cell encapsulation device 100 that includes a membrane or membrane layers 108 with one or more of the flow features (or enhanced bonding features) described above (e.g., one or more of the flow holes, internal slit(s), and recessed perimeters forming end caps). In particular, the membrane layer 108 of the device 100 includes flow holes 102, an internal slit 119, and a recessed outer edge 114. However, in some examples, the device 100 may include only flow holes 102 (and not the internal slit 119 and the recessed outer edge 114), only the recessed outer edge 114 (and not the internal slit 119 and flow holes 102), only an internal slit 119 (or slits, and not the recessed outer edge 114 and flow holes 102), or only two of the three flow features (e.g., the recessed outer edge 114 and flow holes 102, the flow holes 102 and internal slit 119, or the recessed outer edge 114 and internal slit 119).

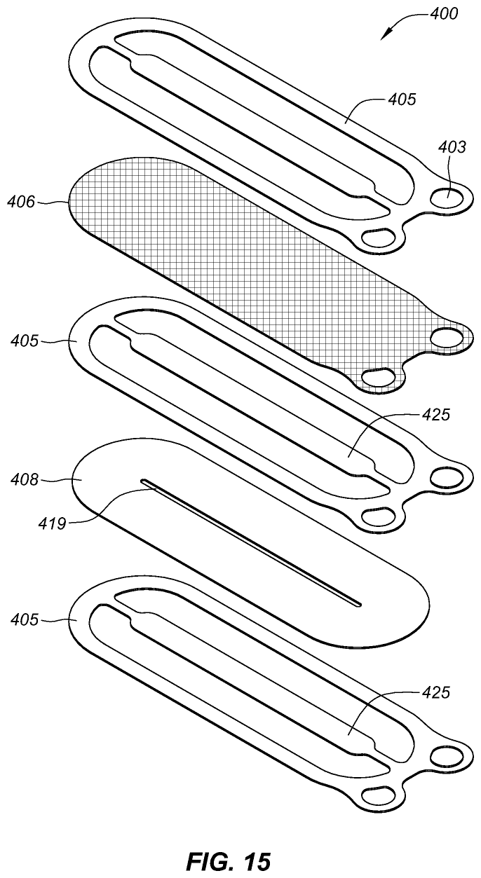

An exemplary cell encapsulation device 400 which includes a membrane or membrane layers including a recessed outer edge and an internal slit (and no flow holes along the perimeter) is shown in FIGS. 15-17B, as described in further detail below.

Turning to FIGS. 4, 5, 7, and 8, the device 100 comprises a plurality of layers that are stacked and bonded (e.g., welded) together to form one or more central chambers 104 (or cavities or lumens) that are configured to receive a therapeutic agent (e.g., cells) therein. The plurality of layers can include two opposing membrane layers 108 that form a membrane with the one or more central chambers 104 defined between the two membrane layers 108.

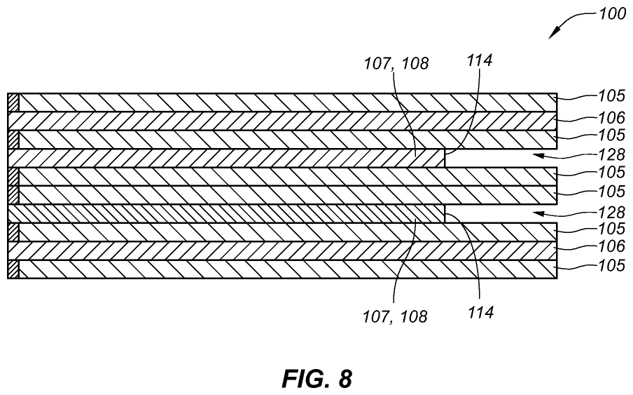

In particular, as shown in the exploded view of FIG. 4 and the stacked (assembled), cross-sectional view of FIG. 8, the device 100 can comprise (from top to bottom in the views of FIGS. 4 and 8): a first film ring 105, a mesh layer 106, a second film ring 105, a non-woven fabric (NWF) layer 107 that is welded (e.g., laser tack welded as described herein) to a membrane layer 108, a third film ring 105, device port tubing 109, a fourth film ring 105, a second membrane layer 108 welded to a second NWF layer 107, a fifth film ring 105, a second mesh layer 106, and a sixth film ring. As used herein, a “film ring 105” can also be referred to as a “film layer 105.”

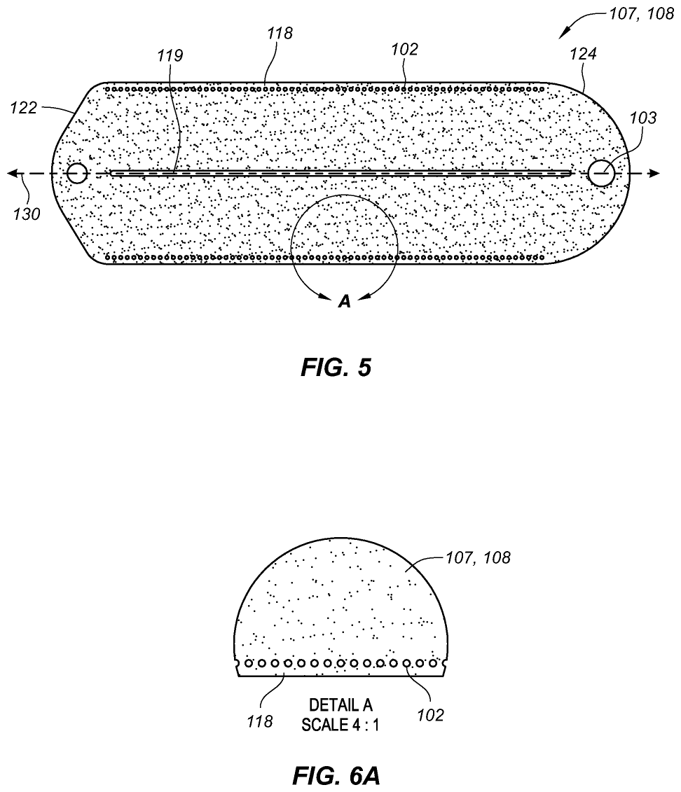

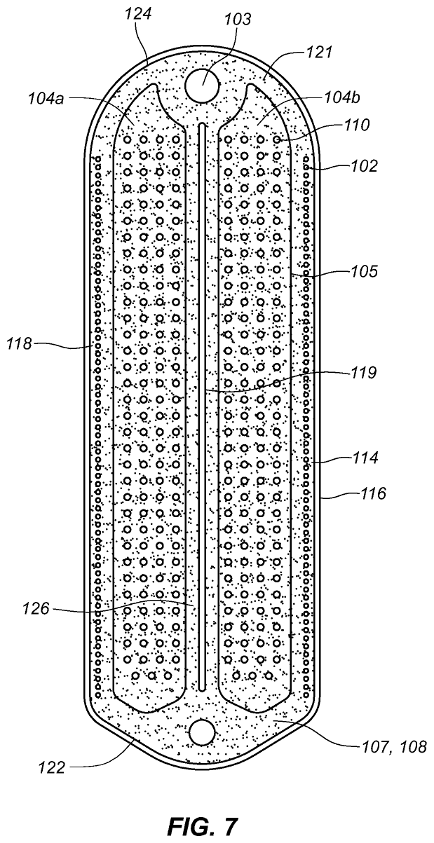

Thus, the device 100 can include six film rings 105 (or film layers). In order to better enable the film rings 105 to melt and bond together during ultrasonic or thermal welding, the membrane 108 (or membrane layers 108) or the membrane and NWF layers 107, 108 are provided with flow holes 102 (FIGS. 5 and 7), an internal slit 119 (FIGS. 4, 5, and 7), and a recessed outer edge or perimeter 114 (which forms an end cap or end caps of the device, as described herein) (FIGS. 7 and 8). For the purposes of illustration only, flow holes 102 are not shown in FIG. 4. However, FIG. 5 depicts the welded together NWF layer 107 and membrane layer 108 with the flow holes 102 and internal weld 119. Additionally, FIG. 7 depicts the welded together NWF layer and membrane layer 107, 108 with an outline of the underlying/overlying film rings 105 provided for reference, thereby showing the recessed outer edge 114 (e.g., the outer edges 118, 122, 124 of the NWF layer/membrane layer 107, 108 relative to the outer edges 116 of the other device layers (e.g., the film rings 105 and mesh layers 106)).

In some examples, as shown in FIG. 5, the flow holes 102 and internal weld 119 extend through both the NWF layer 107 and membrane layer 108.

In some examples, the flow holes 102 and internal weld 119 may extend through only the membrane layer 108.

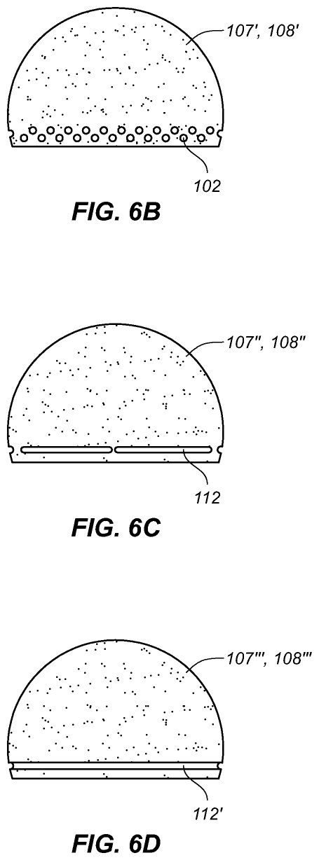

As shown in FIGS. 5 and 7, and the enlarged insert “A” from FIG. 5 shown in FIG. 6A, the flow holes 102 can be round holes spaced apart from one another and arranged in a line along the long edges 118 of the NWF layer/membrane layer 107, 108.

In some examples, the long edges 118 can be opposing long edges of the membrane layer 108 (or welded together NWF layer/membrane layer 107, 108) which are parallel to one another and connected together by curved or non-straight edges 122, 124 (opposing curved edges). In this way, the outer perimeter or recessed outer edge 114 of the NWF layer/membrane layer 107, 108 can be formed by the edges 118, 122, and 124.

In some examples, the long edges 118 can be parallel to a central longitudinal axis 130 of the device 100 which is a longest axis of the device 100 and extends through a center of the device 100 between the non-straight edges 122, 124 (and through the suture holes 103, for example) (FIG. 5).

In some examples, the flow holes 102 can be disposed adjacent but spaced away from the outer perimeter of the NWF layer/membrane layer 107, 108.

In some examples, the flow holes 102 can have a diameter of 0.200 mm with a tolerance maximum of 0.275 mm and tolerance minimum of 0.175 mm.

In some examples, the flow holes 102 can have a spacing, from center-to-center, of 1.0 mm with a tolerance maximum of 1.1 mm and a tolerance minimum of 0.9 mm.

In some examples, instead of round holes, the flow holes 102 can be differently shaped, such as oblong, square, diamond-shaped, rectangular, triangular, or the like.

In some examples, instead of a single row of spaced apart flow holes (e.g., a linear array of flow holes 102) formed along the long edges 118, the NWF layer/membrane layer 107, 108 (or NWF/membrane layer or NWF/non-NWF layer, as defined further below) can comprise two or more adjacent rows of flow holes 102 (as shown in FIG. 6B for the alternative NWF layer/membrane layer 107′, 108′ which can, in some examples, be used in lieu of the NWF layer/membrane layer 107, 108 in the device 100).

As shown in FIG. 6B, in some examples, the flow holes 102 of two adjacent rows can be offset from one another in the direction of the long edges 118.

FIG. 6C shows another alternative NWF layer/membrane layer 107″, 108″ which can, in some examples, be used in lieu of the NWF layer/membrane layer 107, 108 in the device 100 where the flow holes 102 are replaced by a row of spaced apart flow slits 112 (or elongated holes).

FIG. 6D shows yet another alternative NWF layer/membrane layer 107″′, 108″′ which can, in some examples, be used in lieu of the NWF layer/membrane layer 107, 108 in the device 100 where the flow holes 102 are replaced by a single flow slit 112′ extending along each of the long edges 118 (e.g., extending along an entirety of each long edge 118).

As shown in FIGS. 4, 5, and 7, the internal slit 119 can extend through the NWF layer/membrane layer 107, 108.

Alternatively, in some examples, the internal slit 119 can extend only through the membrane layer 108 (and not the NWF layer).

The internal slit 119 extends longitudinally along a central portion of the NWF layer/membrane layer 107, 108, between but spaced away from the non-straight edges 122 and 124. In some examples, the internal slit 119 can extend along a majority of a length of the NWF layer/membrane layer 107, 108, where the length is defined between the non-straight edges 122 and 124.

In this way, the internal slit 119 can be aligned with a longitudinally extending central portion 125 of the film rings 105 (FIG. 4) that is configured to form the internal weld 126 of the device 100 (FIG. 7). As a result, during welding together of the layers of the device 100, the central portion 125 of the film rings 105 can melt through the internal slits 119, thereby melting the central portions 125 of the film rings 105 together and creating a strong internal weld 126.

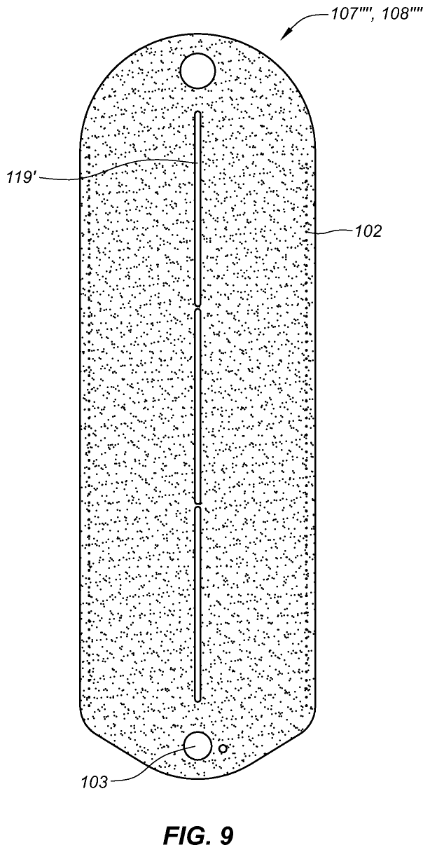

In some examples, instead of one single longitudinally extending slit 119, the membrane layer or the combined NWF layer/membrane layer can have multiple (or a plurality of) longitudinally extending slits 119′ that are spaced apart from one another along the central portion of the NWF layer/membrane layer 107″″, 108″″, as shown in FIG. 9. Though FIG. 9 shows three slits 119′, in some examples, the NWF layer/membrane layer can include a different number of slits 119′, such as two, four, five, or the like.

As shown in FIG. 7 and the cross-sectional view of FIG. 8, the outer edge (outer edges 118, 122, and 124, or perimeter) of the NWF layer/membrane layer 107, 108 can be recessed or offset relative to the perimeter or outer edges of the surrounding layers of film rings 105, thereby creating the recessed outer edge 114 around the perimeter of the NWF layer/membrane layer 107, 108. The recessed outer edge 114 can also be recessed or offset relative to the mesh layers 106.

As used herein, “end cap” or “end caps” refers to the recessed outer edge 114 (formed by edges 118, 122, and 124) of the NWF layer/membrane layer 107, 108 which creates a gap 128 (FIG. 8) that the adjacent film rings 105 can flow through when melted (e.g., during the welding process described herein), thereby melting the film rings 105 to one another to form an end cap and creating a strong perimeter or outer weld 121 (FIG. 7). Thus, as shown in FIG. 8, the perimeter or at least the long edges of the device 100 can have a stepped profile between the film rings 105 and NWF layer/membrane layer 107, 108.

In some examples, the recessed outer edge 114 of the NWF layer/membrane layer 107, 108 can be recessed or offset relative to the perimeter or outer edges of the surrounding layers of film rings 105 by 1.0 mm+/−0.5 mm around the entire perimeter of the device 100.

During assembly of the device 100, the NWF layer 7 can be attached or welded to the membrane layer 108, such as laser tack welded, as described further below.

The various cuts in the membrane layers 108 and/or the welded together NWF and membrane layers 107, 107, such as the flow holes 102, perforations 110, and/or suture holes 103 can be made by laser cutting, for example, or another cutting process.

During assembly of the device 110, the welded together NWF/membrane layer 107, 108 is “sandwiched” between two layers of film (film rings 105). As described above, the end cap is made by recessing the NWF/membrane layer 107, 108 around the perimeter, so that the two film layers 105 that sandwich the NWF/membrane layer 107, 108 melt into each other (film-on-film contact) when the NWF/membrane layer 107, 108 is combined with the remaining layers and welded together.

Any one of the three membrane features described herein (the flow holes 102, end caps formed by the recessed outer edge 114, and internal slit 119), significantly increase the weld strength of the cell encapsulation device. During the welding process (e.g., heat staking or ultrasonic welding), the film or film rings 105 melt through the flow holes 102 located along the perimeter of the NWF/membrane layers 107, 108 and also melts the film or film rings 105 into the interior middle slit 119 of the NWF/membrane layers 107, 108, thereby creating strong internal welds 126 throughout the cell encapsulation device. The outer welds 121 are further strengthened by recessing the NWF/membrane layer to allow film-on-film melting during welding. The film of the film rings 105 (which can comprise Bionate, for example) has a melting temperature that is significantly lower than the other materials in the device, such as the membrane layers 108 (which can comprise PTFE or ePTFE, for example).

Each additional outer most layer of film or film ring 105 on top of the mesh layer 106 can maximize film and mesh intercalation and produce better “anchoring” of the mesh fibers onto the weld.

As used herein “intercalation” can refer to film intercalation into the mesh layer as a result of welding. The extent of film intercalation is determined by the amount of film that surrounds the mesh fibers in the weld. Qualitative assessments, such as visual inspection and SEM imaging, can be used to evaluate film intercalation, as described further below.

The formed internal weld 126 and outer weld 121 create the distinct chambers 104a, 104b defined between the membrane layers 108 (FIG. 7). These chambers 104a, 104b can be configured to receive a therapeutic agent (e.g., cells) therein via respective device port tubing 109 (or device ports 109) that are sandwiched between the opposing film rings 105 and membrane layers 108 and extend from an exterior of the device 100 into the chambers 104a, 104b.

In some examples, as shown in FIGS. 4 and 7, the welded together NWF layer/membrane layer 107, 108 can comprise perforations 110 through both layers (107 and 108). The perforations 110 can be disposed in the areas of the membrane layer 108 that form the central chambers 104a, 104b. In this way, the perforations 110 are different than and separate from the flow holes 102.

The function and design of the perforations 110 are described in more detail below (e.g., see sections entitled “Perforated Cell Encapsulation Devices”, “Perforated Devices Surrounded by a Non-Woven Fabric”, “Diameter of the Perforations”, and “Density of Perforations”).

It should be noted that the texture shown by stippling in the figures (e.g., FIGS. 4-7, 9-14, 18-21B), represents the NWF layer. The stippling is not to be confused with the perforations in the NWF layer and membrane layer (e.g., the perforations 110 shown in FIGS. 4 and 7).

The device 100 can also include one or more suture holes 103. Specifically, the device 100 is depicted in FIGS. 4, 5, and 7 with two suture holes 103, one arranged adjacent to the non-straight edge 124 and one arranged adjacent to the non-straight edge 122. The suture holes 103 can help to align the device layers during assembly and/or implant the device 100 in vivo.

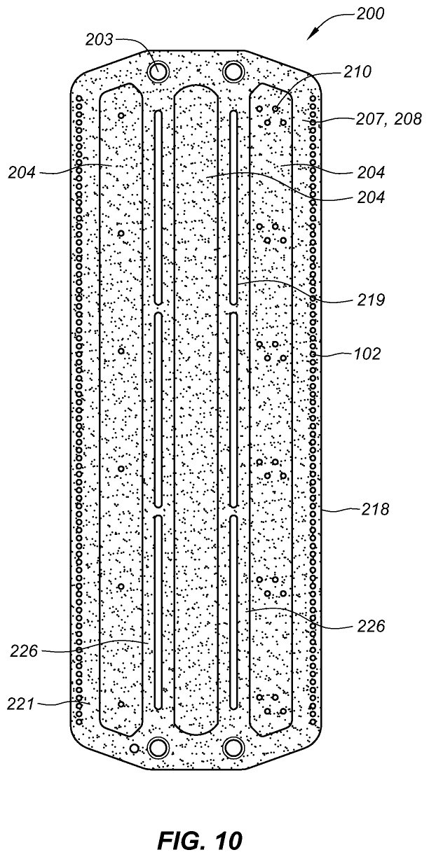

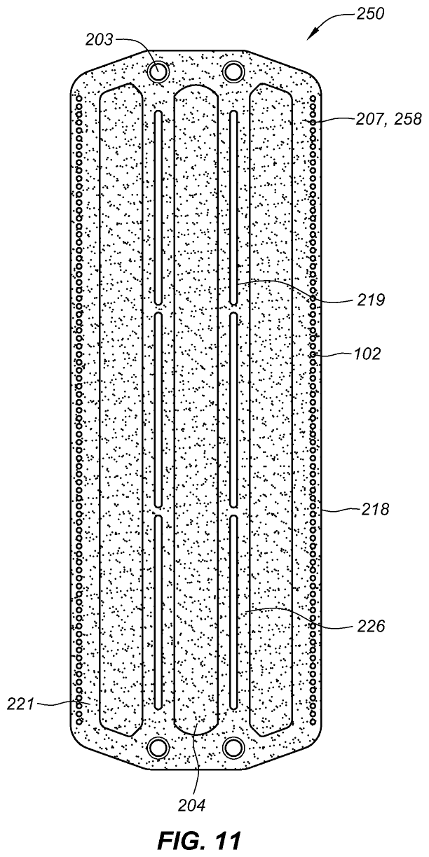

FIGS. 10 and 11 depict plan views of two exemplary cell encapsulation devices 200 and 250, respectively, which have three chambers 204 configured to receive a therapeutic agent (e.g., cells).

Specifically, the cell encapsulation device 200 shown in FIG. 10 includes a membrane layer 208 comprising a linear array of flow holes 102 along its outer long edges 218 (and the outer weld 221). The membrane layer 208 can comprise two rows of spaced apart internal slits 219 (disposed between adjacent chambers 204), which results in six internal slits 219 for the device 200, along the two interior welds 226.

As shown in FIG. 10, the membrane layer 208 comprises an alternate arrangement of perforations 210. For example, FIG. 10 shows a different number and arrangement of perforations 210 in each of the three cell chambers 204 (e.g., none in the central chamber 204 and less in the left chamber 204 than the right chamber 204).

The device 200 is also depicted with four spaced apart suture holes 203.

In some examples, as depicted in FIG. 10, a non-woven fabric layer 207 is welded to the membrane layer 108 and the holes of the membrane layer 208 (e.g., the flow holes 102, perforations 210, and suture holes 203) can extend through the non-woven fabric layer 207 as well.

The cell encapsulation device 250 shown in FIG. 11 is configured similarly to the cell encapsulation device 200, except the membrane layer 258 does not include any perforations in the areas of the cell chambers 204.

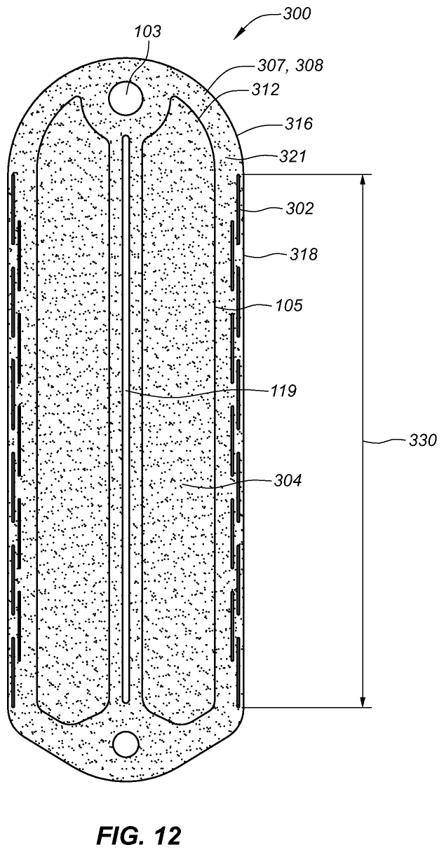

FIGS. 12-14 depict a portion of an exemplary cell encapsulation device 300 that is similar to the cell encapsulation device 100, except its welded together (e.g., tack welded together) NWF layer 307 and membrane layer 308 include a “double slit” configuration of flow slits 302 rather than a single linear array of flow holes 102.

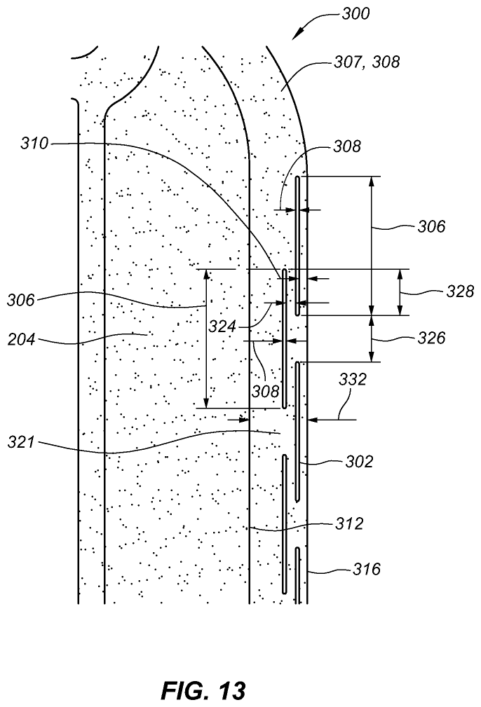

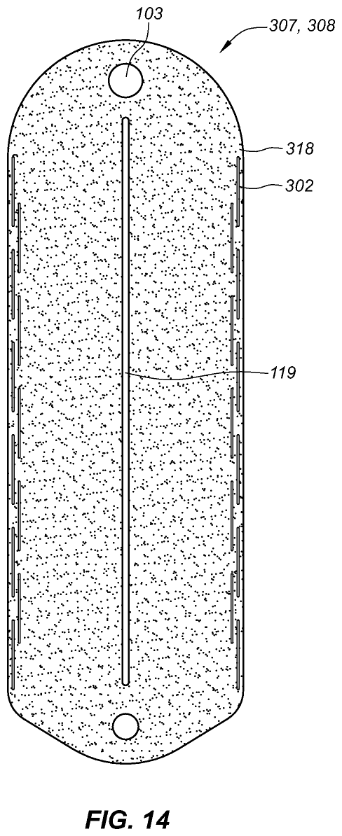

The double slit configuration of flow slits 302 include a plurality of slits 302 arranged along the long edges 318 of the membrane layer 308 into two rows of offset slits 302. Specifically, the two, longitudinally extending rows of flow slits 302 can fit within the area of the outer weld 321 which is defined between the outer perimeter 316 (or edge) of the device 300 and the perimeter 312 of the flow chambers 304 which is depicted in FIG. 12 by the outline of the film rings 105. FIG. 14 shows the NWF layer 307 welded (e.g., tack welded) to the membrane layer 308, with the outline of the film rings removed.

A first row of multiple spaced apart flow slits 302 can be disposed adjacent to the respective long edge 318. A second row of multiple spaced apart flow slits 302 (which is spaced apart from the first row) can be disposed closer to the perimeter 312 of the flow chambers 304 than the first row. The flow slits 302 in the first row are staggered or offset in the longitudinal direction (in the direction of the long edge 318) from the flow slits 302 in the second row.

In some examples, the first and second rows of flow slits 302 can have the same number of flow slits 302.

In some examples, the first row can include more flow slits 302 than the second row. For example, as shown in FIG. 12, the first row can include six flow slits 302 and the second row can include five flow slits 302.

However, in alternate examples, different numbers of flow slits 302 than shown in FIG. 12 is possible.

Though not shown in FIGS. 12-14, the outer edges (including long edges 318) of the membrane layer 308 (or the NWF/membrane layer 307, 308) can be offset from the perimeter of the device 316 to form an end cap (similar to as shown in FIG. 7).

The arrangement of the two rows of offset or staggered flow slits 302 is shown in more detail in the detail view of FIG. 13, which also depicts exemplary dimensions of the flow slits 302.

Each flow slit 302 can have a length 306 and width 308. In some examples, as shown in FIG. 13, the length 306 is longer than the width 308.

In some examples, the width 308 is 0.20 mm.

In some examples, the length 306 is 8.60 mm.

The first row of slits 302 can be spaced away from the outer perimeter 316 of the device 300 by a distance 310. In some instances, the distance 310 is 0.50 mm

The second row of slits 302 can be spaced away from first row of slits 302 by an edge-to-edge distance 324. In some instance, the distance 324 is 0.60 mm.

The slits 302 of the first row of slits can be spaced apart from one another (from end-to-end) by a distance 326. In some instances, the distance 326 is 2.87 mm.

Likewise, in some examples, the slits 302 of the second row of slits can be spaced apart from one another by the distance 326.

The slits 302 of the first row and the second row are staggered with an overlap 328. In some instances, the overlap 328 can be 2.87 mm from end-to-end.

The slits 302 can be symmetrical along the horizontal axis.

The long edge 318 can have a length 330 (FIG. 12). In some examples, the length 330 is 65.74 mm.

A width 332 of the outer weld 321 is depicted in FIG. 13. In some examples, the width 332 is 3.50 mm.

FIGS. 15-17B show an exemplary cell encapsulation device 400 that includes a membrane or membrane layers 408 with an internal slit 419 and recessed outer edge 414. As noted above, the membrane layers 408 of device 400 do not include flow holes.

However, in alternate examples, the device 400 can be configured with flow holes in its membrane layers 408, similar to as shown for device 100.

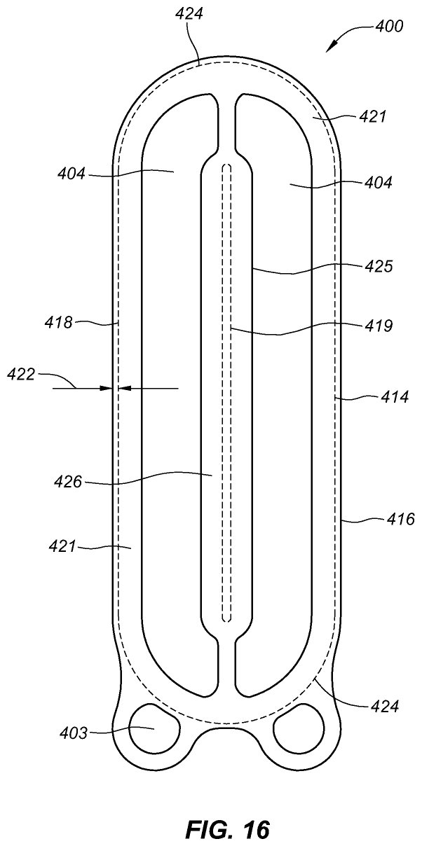

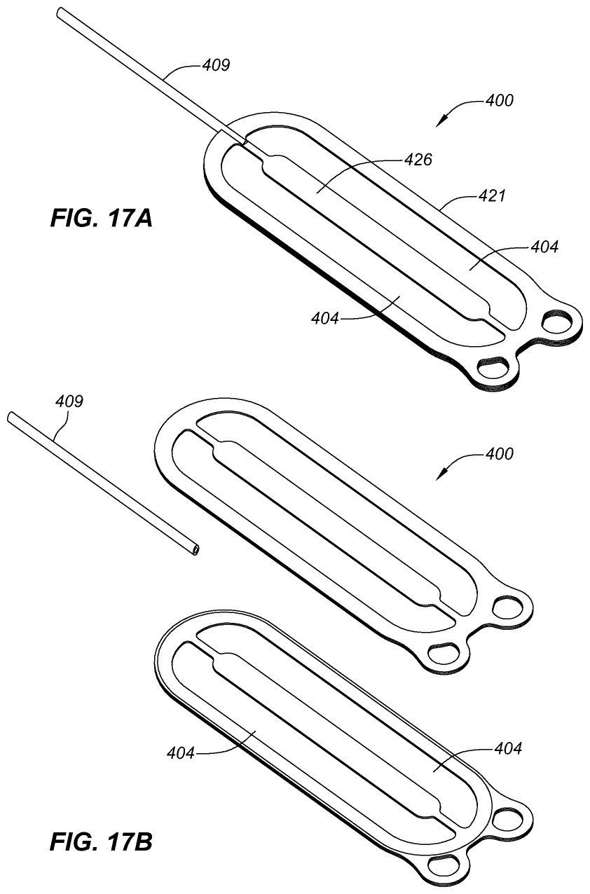

FIG. 15 depicts an exploded view of half of the device 400. The other half of the device 400 is a mirror image of the half shown in FIG. 15. A semi-assembled view of the device 400 is shown in FIG. 17B with the device layers of each half of the device 400 stacked and assembled together, but the two assembled halves shown separated from one another with a device port or device port tubing 409 disposed therebetween. FIG. 17A shows the fully assembled device 400 with the device port tubing 409 extending into the device 400 and sandwiched between the two halves of the device 400. FIG. 16 shows a plan view of the device 400 with the device port tubing 409 removed and an outline of the membrane layer shown in dashed lines to illustrate its recessed outer edge 414 (recessed relative to the outer edge of the remaining layers of the device 400).

The device 400 comprises a plurality of layers that are stacked and bonded (e.g., welded) together to form one or more central chambers 404 (or cavities or lumens) that are configured to receive a therapeutic agent (e.g., cells) therein. The plurality of layers can include two opposing membrane layers 408 that form a membrane with the one or more central chambers 404 defined between the two membrane layers 408 (two chambers 404 are shown in the example of FIGS. 15-17B).

In particular, as shown in the exploded view of FIG. 15, each half of the device 400 can comprise (from top to bottom for the half shown in FIG. 15): a first film ring 405, a mesh layer 406, a second film ring 405, a membrane layer 408, and a third film ring 405. Device port tubing 409 is sandwiched between the two halves, between the middle two film rings 405, as shown in FIGS. 17A and 17B.

As such, the device 400 includes six layers of film rings 405, two mesh layers 406, and two membrane layers 408 that form the membrane with two chambers 404 for receiving the therapeutic agent (e.g., cells) via the device port tubing 409.

In alternative examples, the device 400 can include more or less than two chambers 404 (e.g., one, three, or the like).

The two chambers 404 can be defined by the two membrane layers 408, the outer weld 421, and the internal weld 426 of the device 400.

Similar to the membrane layers 108 of device 100, the membrane layers 408 of the device 400 includes an internal slit 419 extending longitudinally through a middle or center of each membrane layer 408. As a result, the internal slits 419 can align with a central portion 425 of the film rings 405 stacked above and below the membrane layers 408.

Similar to the membrane layers 108 of device 100, the membrane layers 408 of the device 400 have a recessed outer edge 414 (long edges 418 and non-straight edges 424) of the membrane layers 408 which is recessed or offset relative to the outer edge 416 of the remaining device layers (e.g., mesh layers 406 and film rings 405), and thereby forms end caps of the device upon welding of the device layers together.

In some examples the amount of offset 422 between the outer edge 416 of the device and the outer edges (long edges 418 and non-straight edges 424) of the membrane layers 408 (FIG. 16) is 1.0 mm+/−0.5 mm around the entire perimeter of the device 400.

As a result, during welding together of the layers of the device 400, the film rings 405 can melt through the internal slits 419 and across the gaps formed by the recessed outer edge 414 in the membrane layers 408, thereby melting together the stacked film layers 405 and forming end caps and a stronger outer weld 421 and internal weld 426 (relative to a device that does not include end caps and internal slits in the membrane layers).

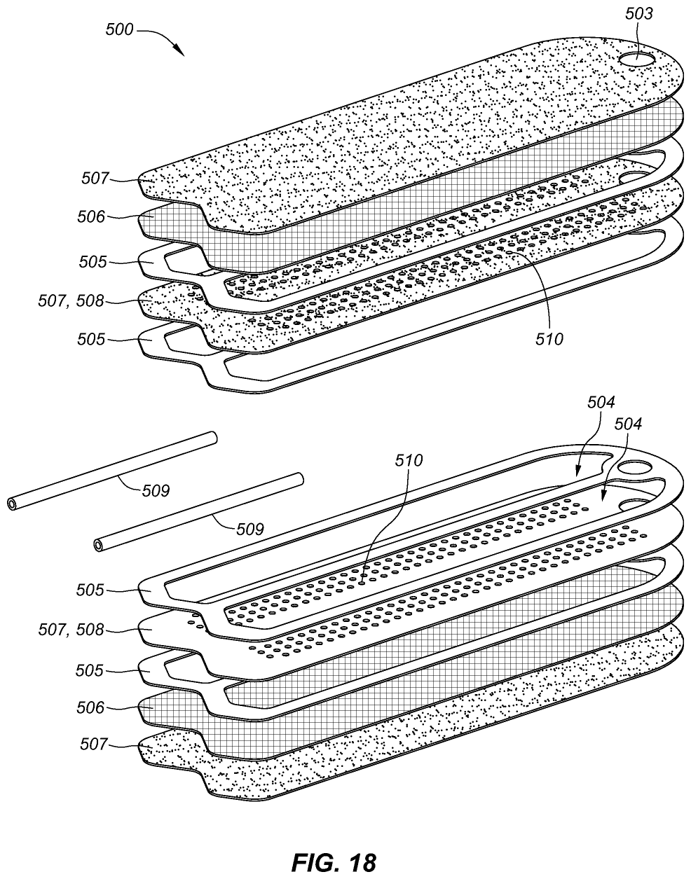

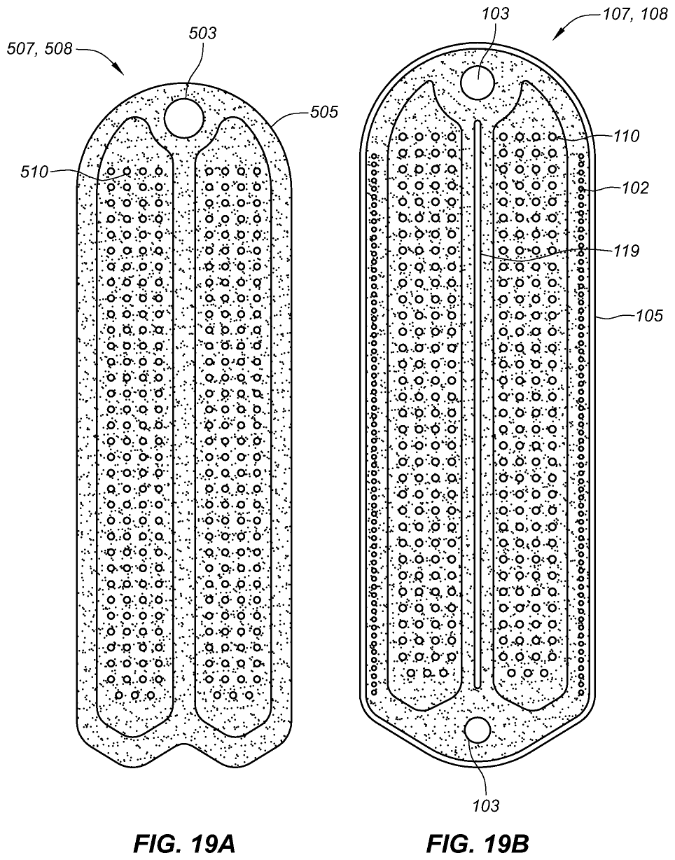

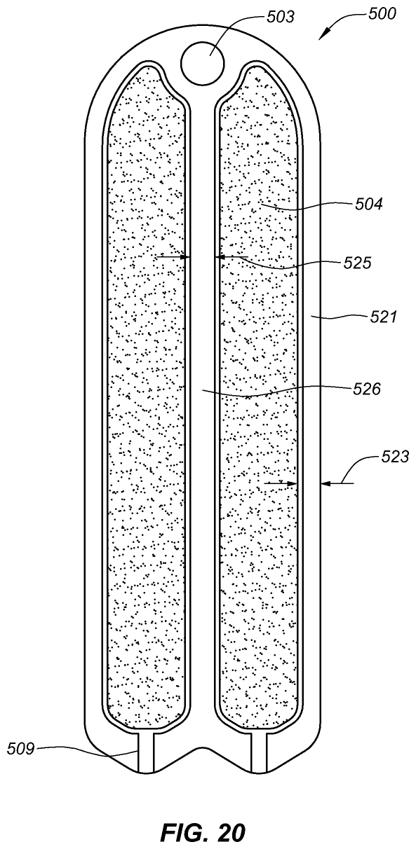

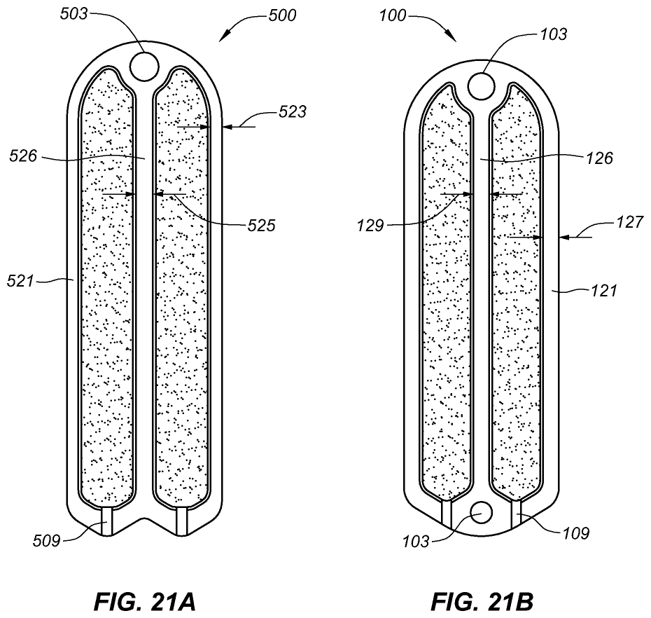

FIGS. 18-21B show an exemplary cell encapsulation device 500 that is similar in stacked structure to the device 100, except its outermost layers are non-woven fabric layers 507 instead of film rings and its membrane layers 508 do not include flow holes, internal slits, or end caps. FIG. 18 shows an exploded view of the device 500, FIG. 19A shows a NWF layer 507 attached to a membrane layer 508 with an outline of the film rings 505 shown for illustration purposes, and FIGS. 20 and 21 show cross-sectional views of the device 500 with alternative weld widths.

The device 500 (which can be referred to as a multi-layered device) comprises a plurality of layers that are stacked and bonded (e.g., welded) together to form one or more central chambers 504 (or cavities or lumens) that are configured to receive a therapeutic agent (e.g., cells) therein. The plurality of layers can include two opposing membrane layers 508 that form a membrane with the one or more central chambers 504 defined between the two membrane layers 508.

In particular, as shown in the exploded view of FIG. 18, the device 500 can comprise (from top to bottom in the view of FIG. 18): a first NWF layer 507, a mesh layer 506, a first film ring 505, a non-woven fabric (NWF) layer 507 that is attached (e.g., heat laminated) to a membrane layer 508 (or the membrane layer 508 is heat laminated to the NWF layer 507), a second film ring 505, device port tubing 509, a third film ring 505, a second membrane layer 508 attached (e.g., heat laminated) to a NWF layer 507, a fourth film ring 505, a second mesh layer 506, and a second NWF layer 507.

In some examples, as shown in FIG. 18, the NWF layer 507 is heat laminated to the membrane layer 508 and the device 500 includes perforations 510 in both layers.

FIGS. 19A and 19B show a side-by-side comparison of the NWF layer 507 attached to the membrane layer 508 of device 500 (FIG. 19A) and the NWF layer 107 attached to the membrane layer 108 of device 100 (FIG. 19B). The outline of the film rings is shown in FIGS. 19A and 19B for illustration.

In some examples, the NWF layer 507 is laser tack welded to the membrane 508. In some examples, the NWF layer 507 is heat laminated to the membrane 508.

In contrast to the device 100, the NWF layer 507 and membrane layer 508 of device 500 do not include flow holes, internal slits, or end caps.

FIG. 20 shows an example of device 500 with different weld widths. In particular, in the example of FIG. 20, the internal weld 526 has an internal weld width 525 of 3 mm. The outer (or external) weld 521 can have an outer weld width 523 of 2 mm.

It should be noted that these weld widths are exemplary and alternate weld width are possible.

For example, FIG. 21A shows an example of device 500 where the internal weld 526 has an internal weld width 525 of 4 mm. The outer (or external) weld 521 can have an outer weld width 523 of 2.5 mm.

In contrast, FIG. 21B shows an example of device 100 where the internal weld 126 has an internal weld width 129 of 4 mm. The outer (or external) weld 121 can have an outer weld width 127 of 3.5 mm. The larger outer weld width 127 for device 100 as compared to the outer weld width 523 of device 500 allows for the perimeter of the membrane layers 108 to be recessed to create the end caps, thereby creating a stronger outer weld 121 (relative to device 500).

As shown in FIG. 21B, device 100 can include two suture holes 103 (instead of the single suture hole 503 of device 100), which can enable the use of an additional alignment pin to improve part alignment during assembly of the device 100.

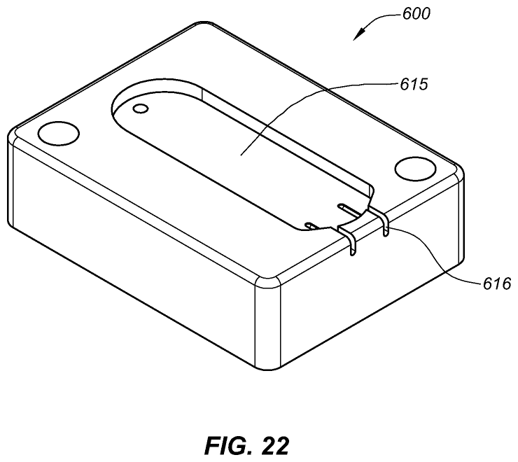

An exemplary welding device, apparatus, or holder 600 for holding the layers of a cell encapsulation device (e.g., any of the cell encapsulation devices described herein, such as the cell encapsulation device 100 or 500) during welding (e.g., ultrasonic welding or heat staking) of all the device layers together (and formation of the device welds) is shown in FIG. 22. The holder 600 can comprise a cavity 615 in which the layers of the device are placed into prior to ultrasonic welding all of the layers together.

In some examples, as shown in FIG. 22, the cavity 615 can include two cavity device port openings 616 in which two tubes (e.g., device port tubing 109 or 509) that are attached to the device can extend through from the inside of the cavity 615 to the outside of the cavity 615.

In some instances, the cavity 615 can be adapted to have more or less than two cavity device port openings 616 (e.g., one, three, or the like), based on the number of the device ports or device port tubing of the cell encapsulation device.

In some instances, the holder 615 can also be used to hold the layers of the device during heat staking (instead of ultrasonic welding) the device layers together to form the assembled cell encapsulation device.

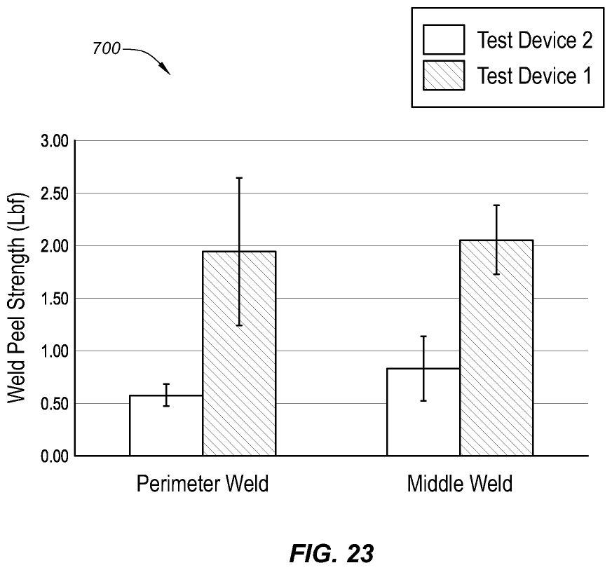

As described further below with reference to FIGS. 23-36, test devices for comparison with one another were constructed. In particular, a device referred to herein as “Test Device 3” was constructed in accordance with the device 100, as described above. However, Test Device 3 did not include perforations 110.

A device referred to herein as “Test Device 1” was constructed in accordance with the device 400, as described above. However, Test Device 1 did not include perforations.

A device referred to herein as “Test Device 2” was constructed in accordance with the device 500, as described above. However, Test Device 2 did not include perforations.

As described above with reference to devices 100, 400, and 500, Test Device 1 comprises a membrane 408, whereas Test Device 2 comprises a NWF layer 507 heat laminated to a membrane 508, and Test Device 3 comprises a NWF layer 107 laser tack welded to a membrane 108.

Test Device 3 has an increased weld width as compared to Test Device 2 around the perimeter (e.g., see FIGS. 21A and 21B). For example, Test Device 3 has an increased weld width from 2 mm to 3 mm (as compared to Test Device 2) to maximize weld strength and provide additional surface area to recess the membrane and increase film on film contact around the perimeter.

In alternate examples, Test Device 3 can have an increased weld width from 2.5 mm to 3.5 mm (as compared to Test Device 2).

FIGS. 23-36 present test data and Scanning Electron Microscope (SEM) images of Test Device 1, Test Device 2, and Test Device 3. A summary of this data is described below and a more detailed description of the various tests and resulting data with these devices is presented further below in the “Examples” section.

FIG. 23 is a bar graph 700 of weld peel strength (LbF) for Test Device 2 versus Test Device 1. The units on the y-axis are weld peel strength (LbF) and the x-axis shows the weld location (perimeter or outer and middle or internal). Graph 700 presents a first bar plot of the peel strength for the perimeter (or outer) weld of Test Device 2, a second bar plot of the peel strength for the perimeter weld of Test Device 1, a third bar plot of the peel strength for the middle (or internal) weld of Test Device 2, and a fourth bar plot of the peel strength for the middle weld of Test Device 1.

As introduced above, Test Device 1 has an end cap and an internal slit in the membrane (or membrane layers), while Test Device 2 does not have an end cap or internal slit.

As shown in graph 700, Test Device 1 has higher weld peel strength (and thus reduced creep failure) at both the perimeter and middle welds than Test Device 2.

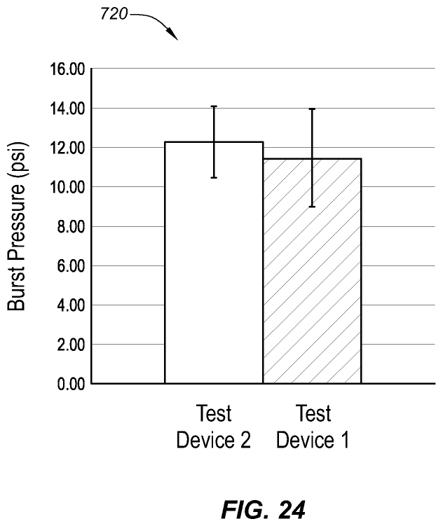

FIG. 24 is a bar graph 720 of burst pressure (psi) for Test Device 2 versus Test Device 1. Burst pressure (in psi) is presented on the y-axis. The graph 720 presents a first bar plot for Test Device 2 and a second bar plot for Test Device 1.

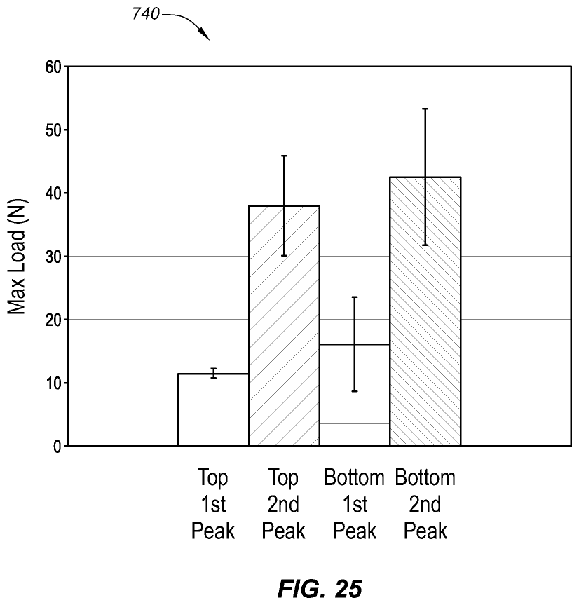

FIG. 25 is a bar graph 740 illustrating mean peel strength (N) of the end cap feature of Test Device 1. The y-axis presents Max Load (N) and the columns along the x-axis depict (from left to right): Top, First Peak; Top, Second Peak; Bottom, First Peak; and Bottom, Second Peak.

The “first peak” and the “second peak” represent the two data points that were taken during the Peel testing: the first peak and second peak. The first peak shows the maximum peel strength of the membrane-to-film bond, and the second peak shows the maximum peel strength of the film-to-film bond, or end cap feature, as explained further below in the discussion of Example 2 in the “Examples” section.

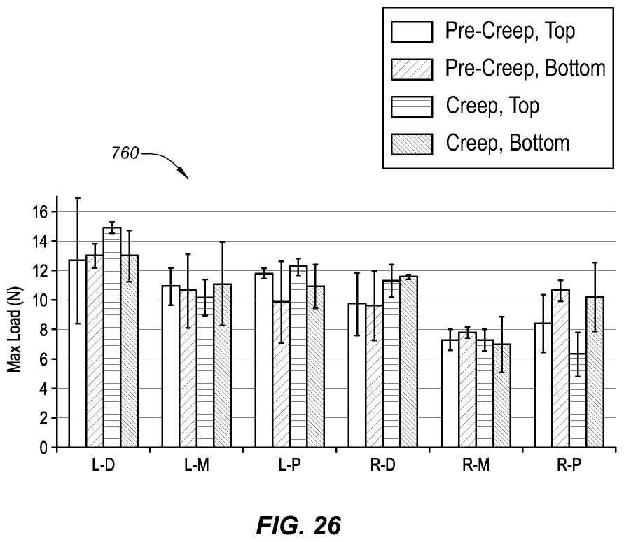

FIG. 26 is a bar graph 760 illustrating peel strength (N) of Test Device 2. The y-axis presents maximum load (N) during peel strength testing. The weld locations that were tested on Test Device 2 are presented on the x-axis. The weld locations include left distal (L-D), left middle (L-M), left proximal (L-P), right distal (R-D), right middle (R-M), and right proximal (R—P). The first bar plot of each cluster of four bars is Pre-Creep, Top; the second bar of each cluster of four bars is Pre-Creep, Bottom; the third bar of each cluster of four bars is Creep, Top; and the fourth bar of each cluster of four bars is Creep, Bottom. Further details on the study used to obtain the data in FIG. 26 is discussed below for Example 1 in the “Examples” Section.

As explained further below in the discussion of Example 2 in the “Examples” Section, from the data, the end cap feature of the Test Device 1 articles (FIG. 25) have stronger peel strength than the membrane-to-film bond in the Test Device 2 articles (FIG. 26).

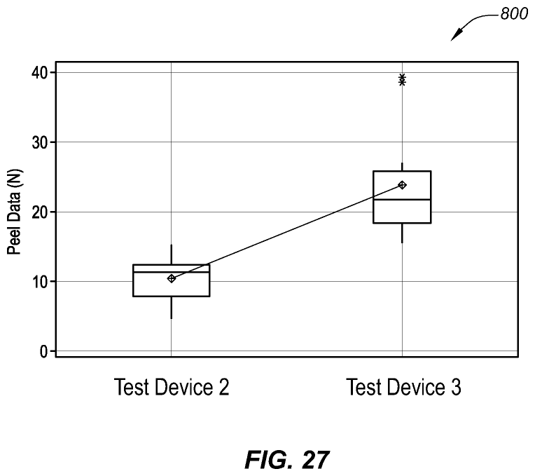

FIG. 27 is a graph 800 of weld peel data (or weld peel strength) (N) of Test Device 2 articles compared to Test Device 3 articles. Weld peel strength (N) of the welds is presented on the y-axis and the test devices are presented on the x-axis. As discussed further below in Example 5 of the “Examples” section, graph 800 shows that Test Device 3 articles had higher peel strength (and thus stronger welds) than the Test Device 2 articles due to the presence of flow holes, end caps, and an internal slit in the NWF layer laser tack welded to the membrane layer. The presence of two additional film rings in the Test Device 3 articles also added to the strength of the welds.

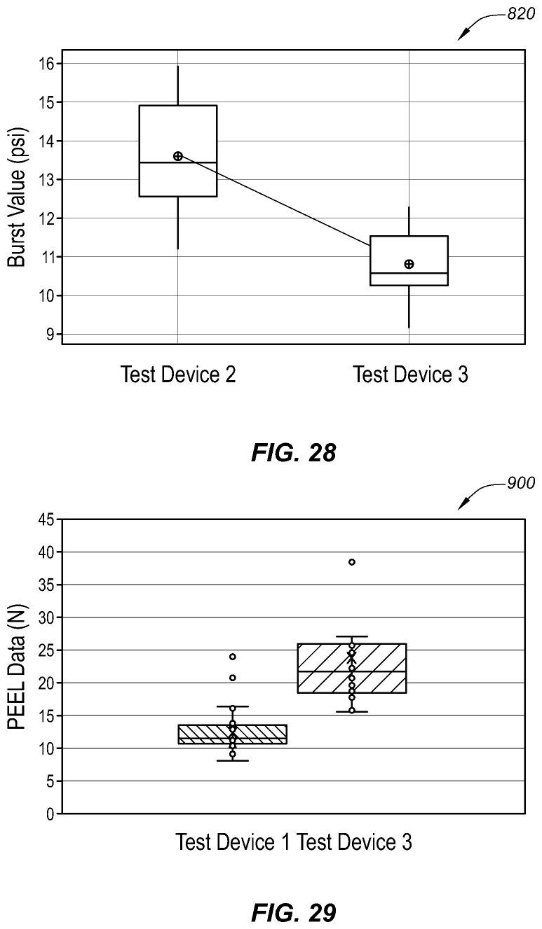

FIG. 28 is a graph 820 of burst pressure (or value) (psi) for Test Device 2 articles compared to Test Device 3 articles. Burt pressure (psi) is on the y-axis and the test devices are presented on the x-axis. As discussed further below in the “Examples” section, both Test Device 2 and Test Device 3 passed set burst strength thresholds for the devices.

FIG. 29 is a graph 900 of weld peel data (or weld peel strength) of Test Device 1 articles compared to Test Device 3 articles. Weld peel strength (N) of the welds is presented on the y-axis and the test devices are presented on the x-axis. As shown in graph 900, Test Device 3 had higher weld peel strength (and thus stronger welds) than Test Device 1 due to the presence of flow holes in the NWF/membrane layers that are tack welded together.

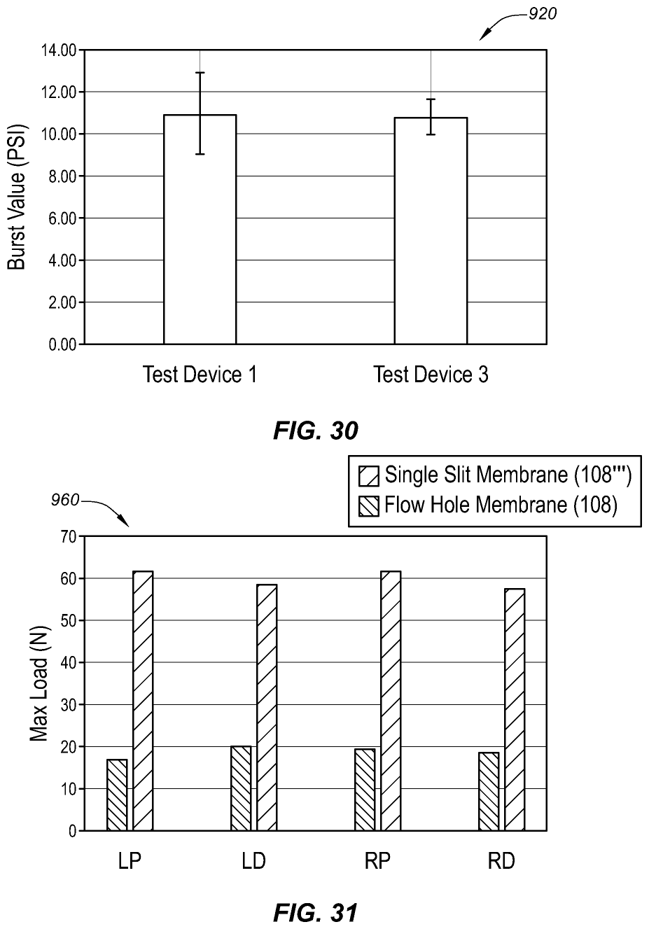

FIG. 30 is a graph 920 of burst pressure (or value) (psi) for Test Device 1 articles compared to Test Device 3 articles. Burt pressure (psi) is on the y-axis and the test devices are presented on the x-axis.

FIG. 31 is a graph 960 of weld peel data (or weld peel strength) for a membrane comprising a single flow slit (such as that shown in FIG. 6D for membrane layer 108″′ with flow slit 112′) compared to a membrane having flow holes (such as membrane layers 108 having flow holes 102, shown in FIG. 5). The max load (N) or weld peel strength of the welds is presented on the y-axis and the weld locations that were tested on the membranes are presented on the x-axis. The weld locations include left proximal (LP), left distal (LD), right proximal (RP), and right distal (RD). As shown in graph 960, the weld peel strength (and thus the strength of the welds) was higher at all locations in the single slit membrane as compared to the flow hole membrane. Thus, a single flow slit, rather than a plurality of spaced apart flow holes, may offer increase weld strength for a cell encapsulation device.

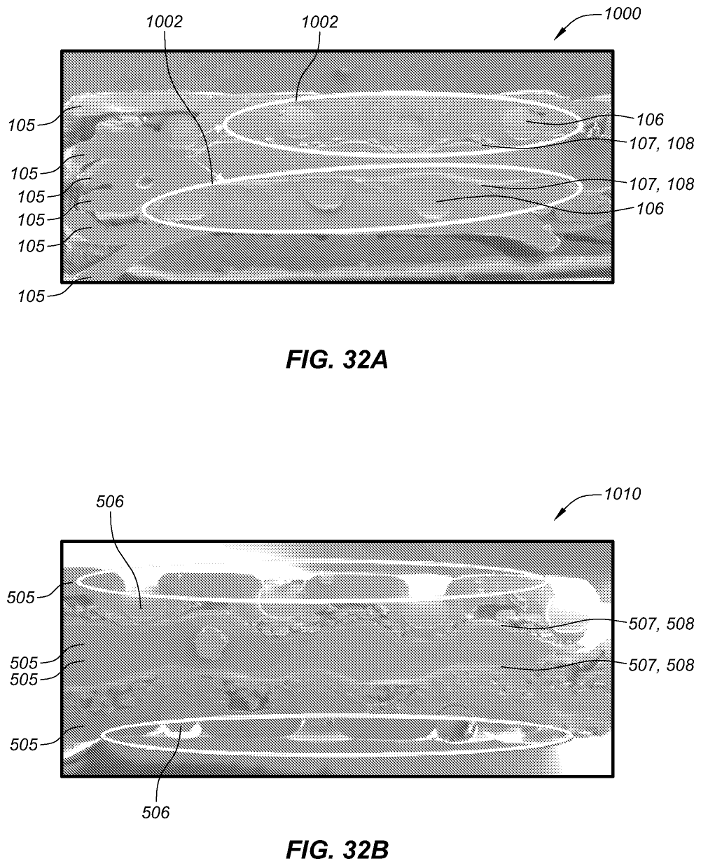

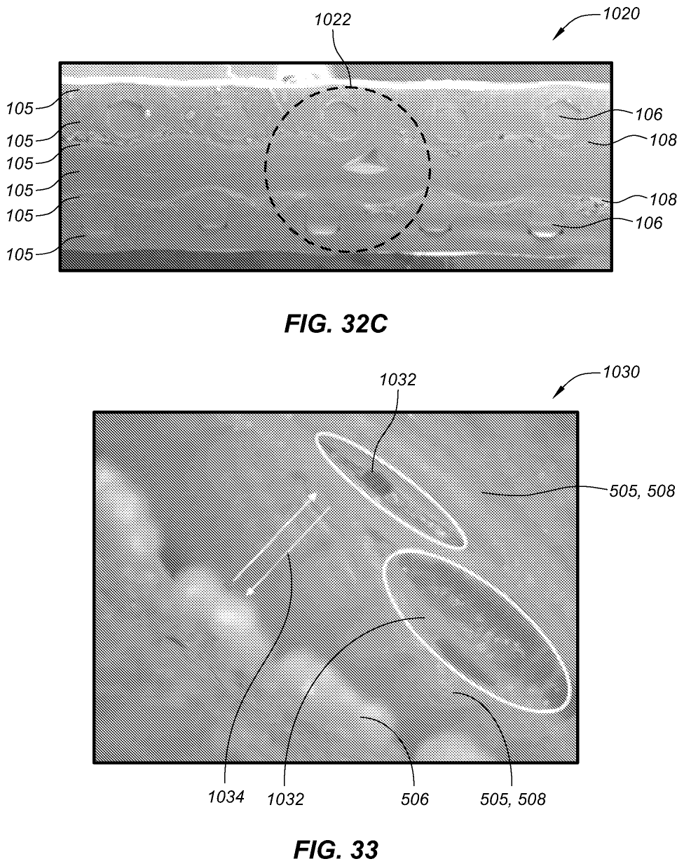

FIG. 32A is a SEM image 1000 of a cross-section of one Test Device 3. The layers for Test Device 3, which correlate to the layers of device 100, are labeled in FIG. 32A. FIG. 32A illustrates a good mesh layer 106/film ring 105/NWF-membrane layer 107, 108 intercalation after ultrasonic welding of the device layers to form the device, as identified with the circles 1002.

FIG. 32B is a SEM image 1010 of a cross-section of a Test Device that is similar to Test Device 2 and device 500 (FIG. 18). The layers for the device shown in SEM image 1010, which correlate to the layers of device 500, are labeled in FIG. 32B. FIG. 32B illustrates a poor mesh layer 506/film ring 505/NWF-membrane 507, 508 intercalation after ultrasonic welding of the device layers to form the device, as indicated by the circles. As shown in FIG. 32B, the film does not surround the mesh fibers of the mesh layers 506, thereby creating a weld with reduced strength as compared to Test Device 3 (FIGS. 32A and 32C).

FIG. 32C is an SEM image 1020 of a cross-section of another Test Device 3. As shown in FIG. 32C, the gap 1022 in the membrane layer 108 created by a flow hole 102 (circled area in FIG. 32C) results in better mesh layer 106/film ring 105/NWF-membrane layer 107, 108 intercalation since the film rings 105 can melt through the flow hole and to one another, thereby creating a strong weld.

FIG. 33 is an SEM image 1030 of a portion of a Test Device 2, which illustrates degradation of the device in the form of broken welds 1032 (circled regions) and mesh delamination 1034 (indicated by two arrows pointing in opposite directions).

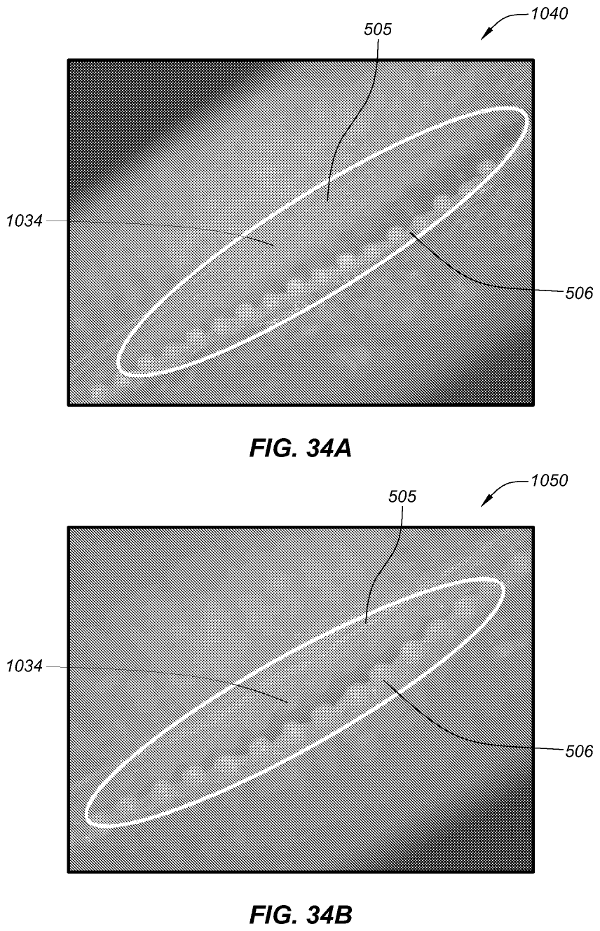

FIGS. 34A and 34B are the first and second SEM images 1040 and 1050, respectively, of a detail portion of Test Device 2, which illustrate degradation of the device by mesh delamination 1034.

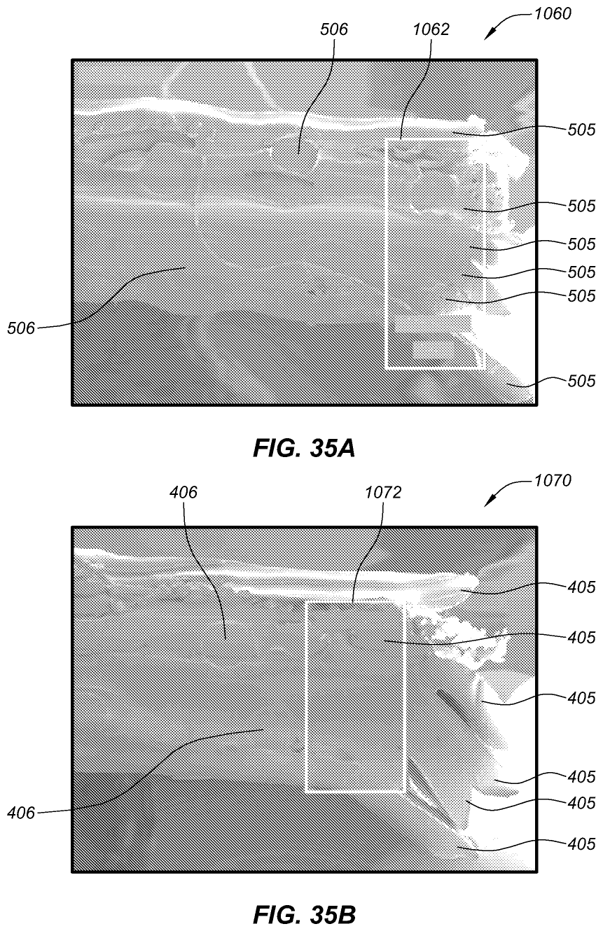

FIG. 35A is an SEM image 1060 of a cross-section of Test Device 2, which does not include the end cap feature. In contrast, FIG. 35B is a SEM image 1070 of a cross-section of Test Device 1, which includes the end cap feature described herein. The region of the outer edge or perimeter of the Test Device 2 which does not include the end cap is outlined by box 1062 in FIG. 35A and the region of the outer edge or perimeter of the Test Device 1 which includes the end cap is outlined by box 1072 in FIG. 35B. There is a better blending of the layers in FIG. 35B, due to the presence of the end caps which allow the film layers 405 to melt together across/cover the end caps.

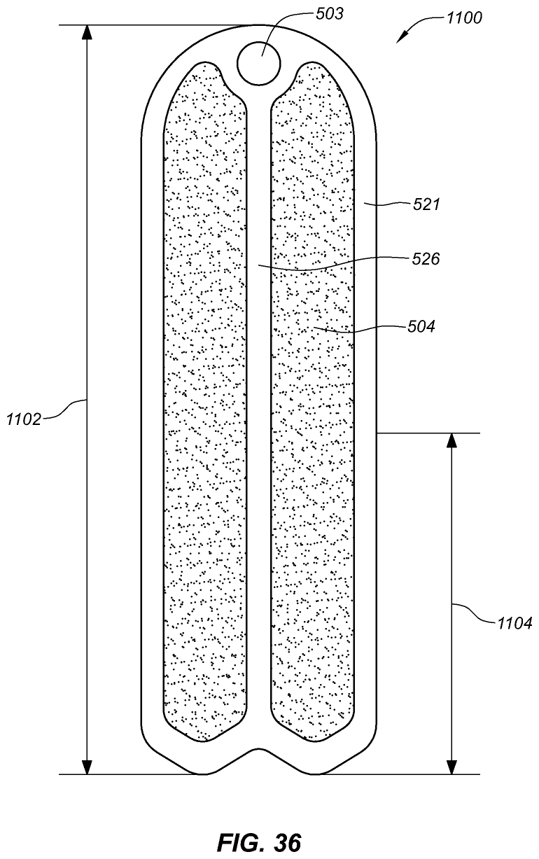

FIG. 36 is a plan view of Test Device 2 1100 which illustrates delamination location measurements for the device. The height 1102 of Test Device 2 1100 is 86.20 mm in the example shown in FIG. 36. The location of the delamination measurement (as described further below for Example 1 of the “Examples” section) is taken at approximately halfway up the right-hand side of the device, as shown by arrow 1104. The bottom of the device is where the device port enters the device. As described above with reference to the device 500, the device has two chambers 504.

Further details on the devices and data described above with reference to FIGS. 1-36 can be found in the following sections.

### Cell Encapsulation Device Layer Configuration Examples

As described above, FIGS. 1-3 are exploded views of certain embodiments of a cell encapsulation device. These figures depict an EN20, which is shorthand for a drug delivery device or cell encapsulation device that has the capacity to support about twenty microliters (20 μl) of implanted cells upon maturation, in an unperforated form. As shown in FIGS. 1-3, the devices can have additional various layers of mesh, film, membrane, and non-woven fabric. Each configuration is manufactured as a “sandwich” that is assembled as a stack of materials and sealed.

Table 1 below describes exemplary cell encapsulation device configurations. Each wall of the device may be comprised of identical number of layers and type of materials, or different number and type of layers depending on the function required and imparted by the layer. The device chamber or housing is created by welding or bonding the periphery and loading the chamber is accomplished by the port tubing. The first row and the bottom row of Table 1 are the layers exposed to or that would be in contact with the host upon implantation. The layers below can be, for example, laminated together, laser tack welded together, ultrasonically welded together or welded together by heat staking. Other methods known to one of skill in the art could also be utilized to weld or bond the layers of the device together.

TABLE 1
Variations in cell encapsulation device materials.

| Device | EN20B1 | EN20B2 | EN20B3 | EN20B4 |
|---|---|---|---|---|
| Material | Non-woven | NWF | Mesh | Mesh |
| Layer | fabric | Film | Film | Film |
| Configuration | Film | Mesh | Heat Laminated | Heat Laminated |
| | Heat Laminated | Heat Laminated | Non-woven | Non-woven |
| | Non-woven | Non-woven | fabric | fabric |
| | fabric (x2) to | fabric | to Cell-Excluding | to Cell-Excluding |
| | Cell-Excluding | to Cell-Excluding | Membrane | Membrane |
| | Membrane | Membrane | Film | Film |
| | Film | Film | Tubing | Tubing |
| | Tubing | Tubing | Film | Film |
| | Film | Film | Heat Laminated | Heat Laminated |
| | Heat Laminated | Heat Laminated | Non-woven | Non-woven |
| | Non-woven | Non-woven | fabric | fabric |
| | fabric x2 | fabric | Membrane | Membrane |
| | Membrane | Membrane | Film | Film |
| | Film | Mesh | Mesh | Mesh |
| | Non-woven | Film | | |
| | fabric | Non-woven | | |
| | | fabric | | |

EN20B1 corresponds to the cell encapsulation device 10 shown in FIG. 1, as described above.
EN20B2 corresponds to the cell encapsulation device 20 shown in FIG. 2, as described above.
EN20B3 corresponds to the cell encapsulation device 30 shown in FIG. 3, as described above.
EN20B4 described in Table 1 has the same configuration as EN20B3 but the density of the NWF layer 7 is different.

In one example, the non-woven fabric and cell-excluding membrane may be laminated, such as using heat lamination or heat press (e.g., an ARB Arbor Press from Plastic Assembly Systems). The press is heated to between about 305-320 Farenheit. A pressure of between 0-6 PSI is applied to the non-woven fabric and membrane at a rate of 3 feet/minute or 10 feet/minute. However, the non-woven fabric and cell-excluding membrane need not be laminated.

In one example, the non-woven fabric layer faces out toward the host while the cell-excluding membrane layer faces in toward the chamber or implanted cells, but a skilled artisan can envision different configurations using the present disclosure, for example, that the non-woven fabric layer can face in toward the chamber or implanted cells while the cell-excluding membrane layer faces out toward the host. In some embodiments, the non-woven polyester fabric is on the outside of the cell-excluding membrane and is laminated or laser tack welded to the membrane.

In some examples, the cell-excluding membrane and/or non-woven fabric are laminated, such as heat laminated, welded, bonded, or laser tack welded together and then perforated. In some examples, the cell-excluding membrane is first perforated and then laminated, such as heat laminated, welded, bonded, or laser tack welded to a non-woven fabric. In some examples, just the non-woven fabric is perforated and then laminated, such as heat laminated, welded, bonded, or laser tack welded to the cell-excluding membrane. When the cell-excluding membrane is perforated the mammalian host is either immunocompromised or treated with immunosuppressant drugs.

The non-woven fabric used in the cell encapsulation device is shown in FIGS. 1-3 as being substantially flat, but it can be further manipulated to increase its thickness or have a variable thickness. For example, the non-woven fabric can be pleated, contoured or embossed. Additionally, fabrics with a plush or pile, such as a looped fabric or tufting as in carpet manufacturing may be utilized to produce a fabric with pile and other three-dimensional structures. See e.g., U.S. Pat. No. 7,754,937 which is herein incorporated in its entirety by reference.

The devices contemplated herein can have many different configurations and different capacities for holding the therapeutic agent. ENCAPTRA EN20 or EN20 or EN20 device or small cell encapsulation device refers to a device with a functional volume of about 20 μl and can contain about 2,500 to 3,500 IEQ of beta cell mass or greater than 80,000 IEQ per kg in a mouse. ENCAPTRA EN250 or EN250 or EN250 device or large cell encapsulation device has a functional volume of about 250 μL and is about 12.5 times (12.5×) greater than the EN20 device and can contain up to about −30,000 to 45,000 IEQ per kg in a mouse. ENCAPTRA EN100 or EN100 or EN100 device has a functional volume of about 100 μL is about 6.5 times (6.5×) greater than the EN20 device and can contain up to about 16,250 to 22,750 IEQ per kg in a mouse. EN-large capacity or EN-LC device is about 48.4 times (48.4×) greater than the EN20. An EN-LC device containing 4 cell chambers, can contain up to about 121,000 to 169,400 IEQ, and so on. Hence, in order for the therapeutic effective dose to be delivered to a patient, it is anticipated that encapsulation using at least about 4, about 5, about 6, about 7, about 8 EN250 devices or about 2 EN-LC devices will be required to deliver sufficient PEC quantities.

In addition to increasing the size of the device to increase the dosing capacity, perforating the device increases the dosing capacity. A perforated EN20 device has a dosing capacity (meaning the beta cell mass achieved at maturation) about 5× that of an unperforated EN20 device. Stated another way dosing of a perforated device is about ⅕ of an intact device.

### Perforated Cell Encapsulation Devices

To promote vascularization shortly after implant, cells are implanted in a perforated cell encapsulation device which provides direct cell-to-cell contact between host vasculature and the encapsulated cells. In some examples, not all the layers of the device are perforated. For example, a perforated cell encapsulation device is provided with perforations in just one layer, for example, the cell-excluding membrane; or, in just the cell-excluding membrane and the non-woven fabric layer. This helps retain the implanted cells/tissue while at the same time allowing exchanges with the host such as ingress of the vasculature, macrophages and the like.

By laser drilling the perforations, the perforation size, number and location can be selected. The perforations are of sufficient size to allow host vascular tissue (such as capillaries) and stromal cells that support pancreatic cell types to enter the device lumen. In one example, the perforations are sized such that host macrophages and other phagocytes can also enter the device and remove necrotic debris from the perforated device lumen. In one embodiment, the perforations are also sized to allow therapeutic agents such as insulin produced by the graft to exit the cell encapsulation device. Perforations allowing for vascular structures to grow into the device lumen help anchor the device to the host and inhibit movement of the device. In one embodiment, the perforations are also sized based on cell aggregate diameter to maximize cell retention.

In some examples, the device includes a cell housing made of a biocompatible material adapted to be implanted in a host, and to substantially contain therapeutic agents which can be immunologically compatible or incompatible with the host, the chamber having a wall comprising cell-excluding membrane and optionally a mesh layer or layers and film weld, said wall having holes traversing just the cell-excluding membrane; where the holes have an inner diameter at the narrowest point large enough to permit a host capillary to traverse the thickness of the wall, and where said holes are numerous enough to permit said host capillary to support the viability of the therapeutic agents which may be contained therein.

In one example, a perforated cell encapsulation device is provided wherein one or more layers of the cell encapsulation device is perforated. In one example, a perforated cell encapsulation device is provided wherein one or more layers of the cell encapsulation device is not perforated. In one example, only the cell-excluding membrane is perforated. In one example, a cell encapsulation device comprises holes which do not traverse each wall of the device is provided. In one example, perforations in the cell encapsulation device consist of holes which do not traverse each wall of the device but host vasculature growth into the inner lumen of the cell encapsulation device still occurs. In one example, a cell encapsulation device that does not comprise a non-woven fabric is disclosed. In one example, a cell encapsulation device that does not comprise a non-woven fabric but the cell-excluding membrane is perforated is disclosed. In such examples, the hole diameter in the cell-excluding membrane is used to retain the cells, i.e., the holes in the device are smaller than the cell aggregates contained therein.

In one example, the cells in the perforated encapsulation device consists of PDX1/NKX6.1 co-positive pancreatic progenitor cells. In one example, the cells in the perforated encapsulation devices consists of immature beta cells expressing insulin (INS) and NKX6.1 or immature beta cells expressing INS, NKX6.1 and MAFB. In one example, the cells in the perforated encapsulation device consists of mature beta cells expressing INS and MAFA or INS, NKX6.1 and MAFA. In one example, the cells in the perforated encapsulation device consists of pancreatic endocrine cells. In one example, the cells in the perforated encapsulation device consists of pancreatic insulin secreting cells. In one example, cells in the perforated encapsulation devices consist of pancreatic beta or insulin cells capable of secreting insulin in response to blood glucose levels.

### Perforated Devices Surrounded by a Non-Woven Fabric

In these examples, the non-woven fabric is on the outside of the cell encapsulation device. Rather than affecting implanted cells, the non-woven fabric enhances host vascularization surrounding the cell housing.

In one example, a cell encapsulation device comprising a non-woven fabric is disclosed. In one example, a cell encapsulation device comprising a non-woven polyester fabric (NWPF) is disclosed. Polypropylene, polyethylene, nylon, polyurethane, polyamide are some examples of a non-woven polyester fabric that can be used. In one example, the cell-excluding membrane is surrounded (or coated) with a non-woven fabric, i.e., the non-woven fabric is external to the cell-excluding membrane. Stated another way, the non-woven fabric faces the host not the implanted cells. In one example, the non-woven fabric forms a jacket around the cell excluding membrane. In one example, only the cell-excluding membrane is perforated, the other layers of the device including the non-woven fabric are not perforated. In one example, just the cell-excluding membrane and the non-woven fabric are perforated, and the other layers of the device are not perforated.

In one example, the holes/perforations are smaller than cell aggregates contained in the device, such as the hPSC-derived aggregates, e.g., definitive endoderm lineage cell aggregates, contained therein. In one example, the holes are smaller than the PDX1-positive pancreatic endoderm cell aggregates contained therein. In one example, the holes are smaller than the pancreatic progenitor cell aggregates contained therein. In one example, the holes are smaller than the pancreatic endocrine cell aggregates contained therein. In one example, the holes are smaller than the mature beta cell aggregates contained therein.

In one example, the hole diameter is small enough to retain the cells but large enough to ensure that the desired therapeutic effect is achieved. For example, in the case of a diabetic patient the hole diameter is determined by the ability of the implanted cells to mature and/or produce insulin in response to blood glucose levels.

In one example, a perforated cell encapsulation device is implanted into a rat or human. In one example, a perforated cell encapsulation device implanted into a rat or human contains perforations in just the cell-excluding membrane and the non-woven polyester fabric (the other layers of the device are not perforated) and wherein the holes are separated by about 2 mm (measuring center to center from the holes) or more and wherein the hole diameter is less than about 100 microns is provided. In one example, a perforated cell encapsulation device implanted into a rat or human contains perforations in just the cell-excluding membrane and the non-woven polyester fabric (the other layers of the device are not perforated) and wherein the holes are separated by about 2 mm or more. In one example, a perforated cell encapsulation device implanted into a rat or human contains perforations in just the cell-excluding membrane and the non-woven polyester fabric (the other layers of the device are not perforated) and wherein the hole diameter is less than about 100 microns is provided.

In one example, a perforated cell encapsulation device implanted into a rat or human contains perforations in just the cell-excluding membrane (the other layers of the device are not perforated) and wherein the holes are separated by about 2 mm or more and wherein the hole diameter is less than about 100 microns is provided.

In one example, a cell encapsulation device comprises a perforated cell-excluding membrane and PDX1-positive pancreatic endoderm cells, which can be implanted into a human patient wherein the PDX1-positive pancreatic endoderm cells mature in vivo to insulin-producing cells. In one example, a cell encapsulation device comprises just a perforated cell-excluding membrane and perforated NWF layer and PDX1-positive pancreatic endoderm cells, wherein the cell encapsulation device can be implanted into a human patient wherein the PDX1-positive pancreatic endoderm cells mature in vivo to insulin-producing cells.

### Diameter of the Perforations

The use of perforated cell encapsulation devices has certain disadvantages such as cellular escape and lesser so, tumorigenicity. The aperture of the perforations should therefore enable the cell-excluding membrane to retain the encapsulated elements, while at the same time allowing exchanges with the host such as ingress of vasculature, macrophages and other phagocytes that can remove necrotic debris from the perforated device lumen and stromal cells that support pancreatic cell types. In one example, the perforations are less than about 100 μm in diameter to allow capillary ingrowth. In one example, the perforations are about 80 μm to about 100 μm, about 80 μm to about 90 m, or about 85 μm to about 90 μm in diameter to allow capillary ingrowth. In one example, the perforations are about 80 m to about 90 μm, about 90 μm to about 100 μm, about 100 μm to about 110 μm, or about 100 μm to about 120 μm in diameter to allow capillary ingrowth. Applicants have previously disclosed that pancreatic progenitor cell aggregates average approximately 180 m in diameter with quartile range approximately 100-200 μm (Schulz et al. (2012) supra), therefore hole diameters of about 100 m or less provide substantial retention of the cell product, while still achieving the other benefits described above and, thus, facilitate both delivery and retrieval of the cells as well as allow capillary ingrowth. Hence, the cells are exposed to the host tissue, e.g., host blood vessels, but due to their larger size, the risk of cell escape is low to de minimus. In one example, the holes have an inner diameter large enough to allow the ingrowth and egress of host capillaries and large enough to allow the hormone produced by the therapeutic agent to exit the device lumen/chamber.

The hole size (diameter) may be varied depending on the cell function. For example, if complete cell containment is not necessary, then there is less restriction with regard to hole diameter and density. The holes in a particular device may have the same diameter or may have different diameters in different parts of the device. For example, if the majority of the encapsulated cells, cell aggregates, organoids, clusters, clumps, and tissues tend to be located approximately in the center of the device, then more holes may be necessary for cell survival in that region of the device as compared to the proximal and distal ends of the device which may have fewer and/or smaller holes. As such, there is a lot of flexibility in the size, density and distribution of perforations, so long as host-implant cell-to-cell vascularization is established shortly after transplantation. Again, FIG. 4, FIG. 7, and FIG. 18, for example, show examples of a perforated device. FIG. 4 shows a double lumen (e.g., two chambers 104) and a double port (e.g., port tubing 109) which reduces areas of cell pooling.

In other examples, pancreatic progenitor cell aggregates which are larger in size as compared to the average hole diameter of the perforation in a device. In one example, the cell encapsulation device is perforated with holes less than about 300 microns, less than about 200 microns, less than about 150 microns, less than about 100 microns, or less than about 75 microns, or less than about 60 microns, or less than about 50 microns in diameter, or less than 40 microns in diameter, or less than 30 microns in diameter. In another example, the cell encapsulation device is perforated with holes with a diameter of about 30 microns to about 500 microns, or about 50 microns to about 300 microns. In one example, the cell encapsulation device is perforated with holes between about 200 to about 50 microns, or about 200 to about 75 microns or about 70 to about 80 microns in diameter. In one example, the hole diameter is greater than about 200 microns. In one example, the hole diameter is about 200 to about 400 microns. In one example, the endoderm lineage cell aggregates are about 50 to about 600 microns in diameter. In another example, the pancreatic lineage cell aggregates are about 50 to about 600 microns in diameter. One of skill in the art would be able to determine the diameter of the aggregates and then determine the required hole diameter of the perforations in a cell encapsulation device such that the aggregates do not escape.

In one example, the perforations have a diameter between about 40 microns to about 150 microns. In one example, the perforations have a uniform diameter. In one example, the perforations do not have a uniform diameter.

### Density of Perforations

In one example, less than 0.4% of the device's surface area is perforated and the holes are separated by about 2 mm (measuring center to center of the holes); however, they can be separated by less or more than 2 mm and still promote host-implant cell-to-cell vascularization. In some examples, less than about 5.0%, less than about 4.0%, less than about 3.0%, less than about 2.0%, less than about 1.0%, less than about 0.8%, less than about 0.3%, less than about 0.2%, less than about 0.1%, less than about 0.05% of the device's surface area is perforated. In some examples about 5.0-0.5%, about 5.0-3.5%, about 4.0-2.0% of the device's surface area is perforated. In some examples, about 0.001% to about 0.2% of the device's surface is perforated. In some examples, about 0.003% to about 0.12% of the device's surface is perforated. In some examples, about 0.55% of the device's surface is perforated. In some examples, about 0.001% to about 0.010%, about 0.010% to about 0.1%, about 0.1% to about 0.2%, or about 0.2% to about 0.3% of the device's surface is perforated.

In one example perforations are avoided by replacing the cell-excluding membrane with a highly permeable membrane. For example, a membrane that consists of 80-120 micron pores in the membrane, such pores occurring at a density much like that described herein.

In one example, a cell encapsulation device comprises a perforated cell-excluding membrane with holes separated by about 0.5 mm, 1.0 mm, 1.5 mm, 2 mm, 4 mm, 8 mm or more (measuring center to center of the holes). In one example, a cell encapsulation device comprises a perforated cell-excluding membrane and perforated NWF layer with holes separated by about 0.5 mm, 1.0 mm, 1.5 mm, 2 mm, 4 mm, 8 mm or more. In one example, a cell encapsulation device comprises a perforated cell-excluding membrane laminated or laser tack welded to a perforated NWF layer with holes separated by about 0.5 mm, 1.0 mm, 1.5 mm, 21 mm, 4 mm, 8 mm or more. In one example, a cell encapsulation device consisting of holes or perforations, wherein the holes are separated by about 0.5 mm-4 mm, or by about 0.5 mm-2 mm, or by about 1.0 mm-2 mm is provided.

The number/density of holes can be from 5-200 or from 20-100 holes per device and will depend in part of the size of the device (lumen surface area). Indeed, the number/density of holes can be from 20 to 50 to 100 holes per device lumen. The number/density of holes can be from 5-200 or from 20-100 holes per device lumen. A skilled artisan can determine the number/density of holes to achieve the desired effect. In the case of a diabetic patient, the number/density of holes is determined by the ability of the implanted cells to mature and/or produce insulin in response to blood glucose levels. The number/density of holes can be from 1 to 200 or from 20 to 135 holes per device.

In one example, a cell encapsulation device comprising holes or perforations, wherein the holes are separated by about 0.5 mm, 1.0 mm, 1.51 mm, 2 mm or more is provided and wherein the hole diameter is less than about 200 microns, less than about 150 microns, less than about 100 microns, or less than about 75 microns. In one example, a cell encapsulation device comprising perforations, wherein the holes are separated by about 2 mm or more and wherein the hole diameter is less than about 200 microns is provided. In one example, a cell encapsulation device comprising holes or perforations, wherein the holes are separated by about 2 mm or more and wherein the hole diameter is less than about 100 microns (measuring center to center of the holes) is provided.

### Large Capacity Devices

Applicants have described various planar and non-planar (e.g., 3-dimensional) implantable devices that are contemplated including but not limited to self-expanding implantable devices, large capacity or macro-encapsulation, planar and non-planar implantable devices, or 3-dimensional macro-encapsulation implantable devices. Other encapsulation implantable devices have been described by Applicant, for example, PCT/US2014/022109, 3-DIMENSIONAL LARGE CAPACITY CELL ENCAPSULATION DEVICE, filed Mar. 7, 2014; and U.S. Design application Numbers: 29/408,366 filed Dec. 12, 2011; Ser. No. 29/408,368 filed Dec. 12, 2011; Ser. No. 29/423,365 filed May 31, 2012; Ser. No. 29/447,944 filed Mar. 13, 2013; Ser. No. 29/484,363, 29/484,359, 29/484,360, 29/484,357; 29/484,356, 29/484,355, 29/484,362 and 29/484,35, titled 3-DIMENSIONAL LARGE CAPACITY CELL ENCAPSULATION DEVICE and filed Mar. 7, 2014.

In one example, the assembly consists of at least 1, 2, 4, 5, 6, 7, 8, 9, 10 or more cell chambers. In another example, the assembly is made such that an assembly can consist of any number of cell chambers (or a modular unit). For example, a modular unit can consist of 1, 2, 3, 4, 5, 6, 7, 8, 9, 10 or more cell chambers, which can depend on the number or dose of cells required for the treatment of the disease. Hence, as used herein, the term “device” can mean a single device consisting of one cell chamber such or one device consisting of multiple cell chambers such as the 3-dimensional device or device assemblies described herein. Thus, in some instances device and assembly can be used interchangeably.

In one example, the devices or assemblies can be fabricated to have a total volume in excess of about 10 μL, 20 μL, 50 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1000 μL or more. The total cell volume can consist of one device with one cell chamber having the desired cell dose or can consist of 1 or more devices or assemblies having any number, or a plurality, of cell chambers which together have the desired cell dose. In one example, the device is improved by creating one or more compartments in the cell chamber as described previously in U.S. Pat. No. 8,425,928.

Devices or assemblies may have certain characteristics which are desirable but are not limited to one or a combination of the following: i) comprises a three-dimensional configuration that allows for delivery of large or high cell doses while at the same time constraining the footprint of the device e.g. space taken up by the device or assembly in the desired anatomical site; ii) comprises folds or bends or angles either in the welds or where the device is sealed or even in the cell chamber, whereby the angle of the folds range from 0 (or 180) to 90 degrees, 0 to 50 degrees, or 0 to 40 degrees.

### Multichamber Modular Devices

In one example, devices or assemblies comprise a plurality or multiplicity of cell chambers interconnected by cell-free zones, e.g., folds and bends. For example, one example comprises multiple porous cell chambers that are laterally connected to each other. In one such example, the multiple porous cell chambers are formed, for example, by ultrasonically welding the top and bottom surfaces of a porous material along a line substantially parallel to a longitudinal axis of the device and forms any of 2, 3, 4, 5, 6, 7, 8, 9, 10, 11, 12, 13, 14, 15, 16, 17, 18 or more cell chambers. Each cell chamber has a fixed volume capacity, e.g., 100 μL, with one or more ports and optionally, an internal matrix scaffold or foam, and, if desirable an internal weld or welds to periodically limit the expansion of the lumen or compartment. In some examples, the cell encapsulation device described herein comprises at least two porous chambers or a sufficient number of chambers to house an adequate human dosage of pancreatic progenitor cells derived from pluripotent stem cells to treat and ameliorate a subject with diabetes once the pancreatic progenitors cells are implanted into the subject. In one example, each chamber has a substantially same inner diameter and can hold about the same number of cells. The availability of multiple chambers allows the use of any number or combination of chambers depending on the volume of cellular preparation required, the disease treatment regimen prescribed, which is within the knowledge and skill of persons skilled in the art to determine. Multichamber devices are disclosed in PCT/US2014/022109, incorporated herein by reference in its entirety.

### Expandable Devices

Conventional implantable devices are commonly made of rigid, non-expandable biocompatible materials. A device described herein can be made of an expandable material or of a non-expandable material. Whether the device is capable of expanding may be an inherent part of the materials employed to make the device, e.g., a polymer sheath which is expandable, or can be designed such that they are expandable or have expandable capabilities.

### Manufacturing Processes

In one example, there is provided a manufacturing process for making one or more devices or assemblies with one or more cell chambers, each chamber can comprise, for example, an outer mesh, a cell-impermeable but porous layer, an adhesive layer or film, and any other component necessary for the device (e.g., the port). Methods of manufacturing devices disclosed herein can include, but are not limited to stamping, welding, casting, molding, extruding, die forming and/or die cutting, and/or cutting (e.g., laser cut, water jet cut, machine tool cut, etc.) each of the layered components of the cell chamber. One or more of the layers can be aligned and stamped or cut together, e.g., by a laser.

Layers of the device can be adhered together by techniques commonly known to one skilled in the art, including but not limited to, thermal caulking, welding (including high frequency or ultrasonic), gluing, taping, pressure heat fusing, and adhesion using pharmaceutically acceptable adhesives, film and the like. In one example, ultrasonic welding is used to join different sheets of a cell chamber or device together, due to its speed, cleanliness (no solvents), production of a thin and narrow seam, and strength.

In another non-limiting manufacturing process, one or more layers of the device can be formed by generating a mechanically drawn and/or computer image of the device or one or more portions of the device. One common commercial software package is AutoCAD, but other drawing engineer software packages are available and can be used.

Manufacturing methods known in the art can be used to produce the disclosed perforated devices. Historically, devices were assembled, loaded with cells, and then a needle was used to manually add perforations to an intact device. As such, all layers of the device were perforated. See U.S. application Ser. No. 12/618,659 and PCT Application No. WO/1993/002635, all of which are incorporated by reference herein in their entireties. Additionally, because the cells were inside the device when the perforations were made some portion of encapsulated cells are in the path of the needle upon perforation and the needle could damage some of the encapsulated cells. This method can also lead to inadvertent contamination (cells leaving the device) as the needle is inserted to make the hole and then removed.

Examples described herein use a laser that provides control over hole size and distribution and does not perforate each layer of the device; and does not perforate the device after the cells are loaded. In this way, no cells are injured or destroyed by forming the perforations, potential contamination is reduced and just the cell-excluding membrane (or just the cell-excluding membrane and non-woven fabric layer) is perforated so that the other layers can help retain the encapsulated cells in the encapsulation device upon implant.

Perforated cell encapsulation devices can be constructed in multiple size configurations such as for preclinical rodent models (with nominal 20 μL capacity) and larger devices for clinical studies (EN250). Perforated and unperforated cell encapsulation devices share identical materials, manufacturing techniques and thickness.

By using lasers instead of a needle, disclosed is the manufacture of perforated cell encapsulation devices wherein only the cell-excluding membrane is perforated. In some examples, the non-woven fabric is laminated, welded, bonded, or laser tack welded to the cell-excluding membrane and only these two layers are perforated. In one example, the manufacture of holes in the device layers is automated.

In one example, the perforations are of circular shape or oval shape or elliptical shape. It should be noted that the perforations can have other shapes such as rectangular or hexagonal or polygonal, or slits. In one example, the perforations have a uniform shape. In one example, the perforations do not have a uniform shape. In one example, the perforations are uniformly distributed on the cell excluding membrane. In one example, the perforations are variably spaced on the cell excluding membrane, for example, they may be clustered at the center of the device or at the ends of the device. In one example, the plurality of perforations is spaced in a series of rows and columns forming a grid arrangement or concentric circles or any other geometric configuration or combinations of such configurations. In one example, the plurality of perforations is randomly distributed. In one example, perforations are not on each cell-excluding membrane but only on one side of the device.

In one example, there are a plurality of different cell populations in the device. In one example, there are a plurality of chambers in the device and each chamber is separated by a cell-free zone or island and each chamber is perforated. In one example, there are a plurality of chambers in the device and each chamber is separated by a cell free zone and not all chambers are perforated. In one example, pancreatic progenitors are encapsulated in one chamber and a different therapeutic agent is encapsulated in another chamber. In this instance, only the chamber comprising the pancreatic progenitors will be perforated.

### Combination Product

The examples described herein disclose a combination product, which refers to a device loaded with cells or therapeutic agent, i.e., each alone may be a candidate medical device or cell product, but used together they make a combination product. In one example, the combination product refers to a perforated device loaded with cells. This is referred to as a “perforated combination product.” The device (perforated or not) can be any macro cell encapsulation device described herein including but not limited to the EN20, EN100, EN250, or the EN-large capacity. The combination product may specify the device size for example VC-01-20 means the EN20 loaded with cells. The cells loaded into the device (perforated or not) may be any cells discussed above including but not limited to definitive endoderm, PDX1-positive endoderm, PDX1-positive foregut endoderm, pancreatic endoderm, pancreatic endoderm cells expressing PDX1 and NKX6.1, endocrine progenitors, endocrine progenitors expressing NKX6.1 and INS, immature beta cell, immature beta cells expressing NKX6.1, INS and MAFB, mature endocrine cells, mature endocrine cells expressing INS, GCG, SST and PP, and mature beta cells and mature beta cells expressing INS and MAFA.

The examples described herein also disclose a combination product, which refers to a device loaded with a therapeutic agent. In one example, the combination product refers to a perforated device loaded with a therapeutic agent. This combination is referred to as a “perforated combination product.” The device (perforated or not) can be any macro cell encapsulation device described herein including but not limited to those cell encapsulation devices as described in U.S. Pat. Nos. 8,278,106 and 9,526,880, PCT Application No. PCT/US2016/0061442 and U.S. Design Patent Nos. D714956, D718472, D718467, D718466, D718468, D718469, D718470, D718471, D720469, D726306, D726307, D728095, D734166, D734847, D747467, D747468, D747798, D750769, D750770, D755986, D760399, D761423, D761424, all of which are incorporated by reference herein in their entireties. The cells loaded into the device (perforated or not) may be any cells discussed herein including but not limited to definitive endoderm, PDX1-positive endoderm, PDX1-positive foregut endoderm, pancreatic endoderm, pancreatic endoderm cells expressing PDX1 and NKX6.1, endocrine progenitors, endocrine progenitors expressing NKX6.1 and INS, immature beta cell, immature beta cells expressing NKX6.1, INS and MAFB, or mature endocrine cells.

A device can be loaded with a cell dose. As used herein, “cell dose” or “dose” generally, is a specific number of cells or cell aggregates or therapeutic agents that are contained in a reservoir or container or vial. A cell encapsulating device can comprise at least one cell chamber, wherein the cell chamber comprises an in vitro population of cells (or a cell dose) comprising, for example, human pancreatic and duodenal homeobox gene 1 (PDX1)-positive pancreatic progenitor cells at a dose capable of producing a graft containing an islet equivalent (IEQ) of 2,500 to 1,000,000. For example, the IEQ produced can be 100,000 to 300,000, 100,000 to 200,000, or 200,000.

As used herein, the term “loading” means filling or putting something into something else, e.g., filling or loading a device with cells or an agent, or filling or loading a tube with cells or an agent.

Perforated encapsulation devices loaded with pancreatic endoderm cells (“perforated combination product”) which mature when implanted in vivo are intended to reduce insulin dependence and/or reduce hypoglycemia in high-risk type I diabetic patients who are hypoglycemia unaware, labile (brittle), or have received an organ transplant and who can tolerate, or are already on, immune suppression therapy. The primary method of action is via human pancreatic endoderm cells (PEC) or pancreatic progenitor cells, contained in a permeable, durable, implantable medical device that facilitates direct host vascularization. The PEC cells differentiate and mature into therapeutic glucose-responsive, insulin-releasing cells after implantation. As such, the perforated combination product supports secretion of human insulin. The perforated combination product limits distribution (egress) of PEC cells in vivo. The perforated combination product will be implanted in a location that permits sufficient vascular engraftment to sustain the population of therapeutic cells within the device and facilitate distribution of insulin and other pancreatic products to the bloodstream. The perforated combination product is intended to be implanted and explanted with conventional surgical tools, and to provide a therapeutic dose for two years or more. The device is intended to retain an adequate dose of the PEC cell product during formulation, shelf-life, handling and surgical implant to achieve clinical efficacy and ensure the cell product is located within the tissue capsule to meet safety requirements.

The perforated combination product is comprised of a Perforated Device (PD) containing a dose of PEC, a human pancreatic progenitor cell therapy product. After implantation into a patient, the perforated combination product is designed to enable device integration and direct vascularization of the implanted cell product, to permit differentiation and maturation of PEC cells into glucose-responsive, insulin-producing cells for treatment of insulin-requiring patients.

A perforated device (PD) is defined as a durable, biocompatible, easily-removable implant device comprised of stacked material layers that are bonded together to form a cell-containing lumen. The device is comprised of biocompatible and biostable materials intended for long-term implantation. A semi-permeable membrane permits diffusion of nutrients to the lumen immediately post-implantation to sustain implanted cell viability, while in parallel, perforations in the membrane enable growth of host blood vessels into the device lumen and directly to the implanted cells, improving perfusion and release of implanted cell products, including insulin, into the bloodstream.

Because the PD contains perforations large enough to allow invasion or ingress of host blood vessels, other host cells will migrate into the device's cell-containing lumen, including immune cells, necessitating the use of immune suppression medications.

The perforated combination product is expected to be implanted for a period of five years, but is required to meet its intended use for at least two years. The design intent of the PD is to provide a defined, protected space for early survival and differentiation/maturation of implanted cells during the period of capsule formation, and retain the bulk of such cells throughout the period of engraftment. Device components must be biocompatible. Two device configurations are being developed for clinical study: a device with sufficient volume to potentially achieve therapeutic dosing, and a smaller unit suitable for easy implant and explant to assess engraftment and host tissue response via histology at intermediate time points (sentinel). The size and number of perforations in the device should be chosen to allow ingress of adequate quantities of host blood vessels directly into the implanted cells, without impairing the ability of the perforated device to retain adequate cell dose to provide efficacy. Further, the perforated device shall ensure that an adequate quantity of implanted cells is removed from the body during product explant, which will include surrounding host tissue capsule, to satisfy safety requirements.

The design of the PD and perforated combination product have additional benefits related to their similarities to the intact (without holes) cell encapsulation device.

The PD leverages the same materials, similar manufacturing processes, and the extensive biocompatibility testing established for the intact cell encapsulation devices previously disclosed by Applicants in U.S. Pat. No. 8,278,106 and U.S. application Ser. No. 14/201,630.

Since the intact and perforated devices share similar geometry and handling characteristics, the surgical procedures for both implant and explant are intended to be the same for both products. In summary, the perforated combination product is designed to leverage existing manufacturing processes of, and clinical experience with, the intact cell encapsulation devices for cell product delivery.

Other embodiments are described with reference to the numbered paragraphs below:

Embodiment 1. A cell encapsulation device comprising a non-woven fabric.

Embodiment 2. The cell encapsulation device of embodiment 1, further comprising a cell-excluding membrane wherein the non-woven fabric is external to the cell-excluding membrane.

Embodiment 3. A cell encapsulation device comprising a cell-excluding membrane and a non-woven fabric external to the cell-excluding membrane wherein only the cell-excluding membrane is perforated.

Embodiment 4. The cell encapsulation device of embodiment 3, wherein host blood vessels come in direct contact with a lumen of the cell encapsulation device.

Embodiment 5. A cell encapsulation device comprising a cell-excluding membrane and a non-woven fabric external to the cell-excluding membrane wherein only the non-woven fabric is perforated.

Embodiment 6. The cell encapsulation device of embodiment 5, wherein host blood vessels come in direct contact with the outer surface of the cell encapsulation device.

Embodiment 7. A cell encapsulation device comprising a cell excluding membrane, a non-woven fabric external to the cell-excluding membrane and either a mesh layer, film weld or both wherein the non-woven fabric and cell-excluding membrane are perforated.

Embodiment 8. The cell encapsulation device of embodiment 7, wherein host blood vessels come in direct contact with a lumen of the cell encapsulation device.

Embodiment 9. A cell encapsulation device comprising a cell-excluding membrane and a non-woven fabric external to the cell-excluding membrane wherein only the non-woven fabric and cell-excluding membrane are perforated.

Embodiment 10. The cell encapsulation device of embodiment 9, wherein host blood vessels come in direct contact with the outer surface of the cell encapsulation device.

Embodiment 11. The cell encapsulation device of embodiment 9, wherein host blood vessels form entirely through the cell encapsulation device and come in direct contact with a therapeutic agent loaded into the cell encapsulation device.

Embodiment 12. The cell encapsulation device of embodiment 9, wherein the non-woven fabric is laminated or tack laser welded to the cell excluding membrane.

Embodiment 13. The cell encapsulation device of embodiment 9, wherein the cell encapsulation device is implanted into a mammalian host treated with at least one immunosuppressant drug.

Embodiment 14. The cell encapsulation device of embodiment 13, wherein the immunosuppressive drug is selected from the group consisting of calcineurin inhibitors, anti-metabolite immunosuppressives, and combinations thereof.

Embodiment 15. The cell encapsulation device of embodiment 13, wherein the immunosuppressive drug is selected from the group consisting of Cyclosporine A (CsA), Mycophenolate Mofetil (MMF), Tacrolimus (TAC) and combinations thereof.

Embodiment 16. A cell encapsulation device comprising a perforated non-woven fabric implanted into a host treated with immunosuppressive drugs.

Embodiment 17. A cell encapsulation device comprising a non-woven fabric outside a cell-excluding membrane implanted into a host treated with immunosuppressive drugs.

Embodiment 18. The cell encapsulation device of embodiment 16 or 17, wherein the non-woven fabric is perforated.

Embodiment 19. The cell encapsulation device of embodiment 17, wherein the cell-excluding membrane is perforated.

Embodiment 20. The cell encapsulation device of embodiment 17, wherein the cell-excluding membrane and non-woven fabric are perforated.

Embodiment 21. The cell encapsulation device of embodiment 16 or 17, wherein the immunosuppressive drug is selected from the group consisting of calcineurin inhibitors, anti-metabolite immunosuppressives, and combinations thereof.

Embodiment 22. The cell encapsulation device of embodiment 21, wherein the immunosuppressive drug is selected from the group consisting of Cyclosporine A (CsA), Mycophenolate Mofetil (MMF), Tacrolimus (TAC) and combinations thereof.

Embodiment 23. A cell encapsulation device, comprising a cell-excluding membrane and a non-woven fabric, wherein the non-woven fabric is laminated, welded, bonded, or tack laser welded to the cell excluding membrane.

Embodiment 24. A method for promoting survival of cells transplanted in vivo in a mammal, said method comprising: a) loading cells into a perforated cell encapsulation device; and b) implanting the perforated device containing cells into a mammalian host thereby promoting cell survival of transplanted cells.

Embodiment 25. The method of embodiment 24, wherein the cells are pancreatic endoderm cells.

Embodiment 26. The method of embodiment 24, wherein the mammal is not a mouse.

Embodiment 27. The method of embodiment 24, wherein the mammal is a human or rat.

Embodiment 28. A method of lowering blood glucose in a mammal comprising: 1) loading cells into a cell encapsulation device wherein the device comprises a perforated cell-excluding membrane and a perforated non-woven fabric external to the cell-excluding membrane and no other perforated layers; b) implanting the cell encapsulation device into a mammalian host; and c) maturing the implanted cells thereby lowering blood glucose in a mammal.

Embodiment 29. A cell encapsulation device comprising a cell-excluding membrane and a non-woven fabric external to the cell-excluding membrane wherein the non-woven fabric is laminated, welded, bonded, or tack laser welded to the cell-excluding membrane.

Embodiment 30. The cell encapsulation device of embodiment 29, wherein the non-woven fabric and the cell-excluding membrane are perforated.

Embodiment 31. A cell encapsulation device comprising a cell-excluding membrane and no NWF wherein only the cell-excluding membrane is perforated.

Embodiment 32. A cell encapsulation device comprising a cell-excluding membrane and no NWF implanted into a mammal treated with an ISD wherein only the cell-excluding membrane is perforated.

Embodiment 33. A cell encapsulation device comprising a cell-excluding membrane and no NWF implanted into a rat or human treated with an ISD wherein only the cell-excluding membrane is perforated.

Embodiment 34. A cell encapsulation device comprising a cell-excluding membrane and no NWF implanted into a mammal not treated with an ISD wherein only the cell-excluding membrane is perforated.

Embodiment 35. A cell encapsulation device comprising a cell-excluding membrane and no NWF implanted into a rat or human not treated with an ISD wherein only the cell-excluding membrane is perforated.

Embodiment 36. A cell encapsulation device comprising a cell-excluding membrane and NWF wherein only the cell-excluding membrane and NWF are perforated.

Embodiment 37. A cell encapsulation device comprising a cell-excluding membrane and NWF implanted into a mammal treated with an ISD wherein only the cell-excluding membrane and NWF are perforated.

Embodiment 38. A cell encapsulation device comprising a cell-excluding membrane and NWF implanted into a rat or human treated with an ISD wherein only the cell-excluding membrane and NWF are perforated.

Embodiment 39. A cell encapsulation device comprising a cell-excluding membrane and NWF implanted into a mammal not treated with an ISD wherein only the cell-excluding membrane and NWF are perforated.

Embodiment 40. A cell encapsulation device comprising a cell-excluding membrane and NWF implanted into a rat or human not treated with an ISD wherein only the cell-excluding membrane and NWF are perforated.

Embodiment 41. A cell encapsulation device comprising an intact cell-excluding membrane and no NWF.

Embodiment 42. A cell encapsulation device comprising an intact cell-excluding membrane and no NWF implanted into a mammal treated with an ISD.

Embodiment 43. A cell encapsulation device comprising an intact cell-excluding membrane and no NWF implanted into a rat or human treated with an ISD.

Embodiment 44. A cell encapsulation device comprising an intact cell-excluding membrane and no NWF implanted into a mammal not treated with an ISD.

Embodiment 45. A cell encapsulation device comprising an intact cell-excluding membrane and no NWF implanted into a rat or human not treated with an ISD.

Embodiment 46. A cell encapsulation device comprising an intact cell-excluding membrane and an intact NWF.

Embodiment 47. A cell encapsulation device comprising an intact cell-excluding membrane and an intact NWF implanted into a mammal treated with an ISD.

Embodiment 48. A cell encapsulation device comprising an intact cell-excluding membrane and an intact NWF implanted into a rat or human treated with an ISD.

Embodiment 49. A cell encapsulation device comprising an intact cell-excluding membrane and an intact NWF implanted into a mammal not treated with an ISD.

Embodiment 50. A cell encapsulation device comprising an intact cell-excluding membrane and an intact NWF implanted into a rat or human not treated with an ISD.

### Pre-Healed or Pre-Vascularized Devices

A cell encapsulation device as disclosed herein can be placed in the host tissue already filled or loaded with a therapeutic agent, e.g., cells. However, a cell encapsulation device as disclosed herein can also be placed in the host tissue, prior to being filled with a therapeutic agent, such a device is called a “pre-vascularized” device or a “pre-healed” device. The device is incubated in the host body for a time sufficient to allow for vascularization. After host vascularization occurs, the device is then loaded with a therapeutic agent, or cells.

For example, an empty device is transplanted into a host body until the device is infiltrated with vascular and connective tissue, then the device is accessed through a surgical incision, and the device is loaded with a desired cell population through, for example, a device port. After the device is loaded the device port is sealed.

### Modes of Administration

The device containing a therapeutic agent (e.g., cells), can be implanted into a subject (e.g., mammal, human, dog, or cat) in a variety of locations. One skilled in the art will recognize what anatomical location(s) will be suitable for the device implants, depending upon the therapeutic agent. For example, cells contained within a device may be implanted subcutaneously anywhere in the body, such as the trunk and limbs, or internally, such as in the omentum, intraperitoneal cavity, peritoneal wall, or pre-peritoneum. The chosen implant site will facilitate therapeutic agent (e.g., cell) survival by providing adequate supply of oxygen and nutrients, while also enabling hormones such as insulin that are secreted by the cells to be delivered to the subject. During and following the implant procedure, anesthesia, paralytics, benzodiazepines, antibiotics, analgesics, anticoagulants, antiemetics, and anti-inflammatory agents may be given to the subject for pain control, infection prevention and to promote healing. Each subject will be monitored for the desired therapeutic outcome; if necessary additional doses of cells contained in a device can be administered to achieve the desired magnitude and duration of effect, or the device implants comprising the cells may be partially or fully retrieved once a therapy is no longer required or in the event of an adverse outcome.

Alternatively, cells used for implant may be genetically modified with one or more kill switches, i.e., genetic manipulations that confer select sensitivity to a particular type of drug that can be administered to the subject to induce toxicity in the implanted cells. Such genetic kill switches are known to one skilled in the art and include the herpes simplex virus I-derived thymidine kinase (HSV-TK) gene and the use of ganciclovir (or analogs) as a pro-drug to activate HSV-TK, or a transgene expressing a proapoptotic molecule such as modified caspase 9 fused to a FK506 binding protein (FKBP) to allow conditional dimerization using a small molecule pharmaceutical.

### Tacking, Sealing or Welding of a Device

In some examples, the cell encapsulation device or assembly comprises at least one, or at least two cell chambers. The cell encapsulation device or assembly is formed by tacking, welding, and/or sealing the device peripherally and/or internally to wholly enclose the cells in the cell chamber made therein. Depending on the context, “tacking”, “welding”, “enclosing” and/or “sealing” are used interchangeably and one skilled in the art will appreciate their meanings based on their use. A number of techniques are used for welding plastics and any of them are contemplated in this disclosure as a means to seal the cell chambers, devices, assemblies, or the layers of the cell chambers, devices, or assemblies together. For example, although the devices herein can use high frequency ultrasonic welding, adhesive, and clamps, other plastic welding methods are contemplated including but not limited to hot gas welding or hot air welding using a heat gun that produces a jet of hot air that softens both the parts to be joined and a plastic filler rod; hot air/gas welding; heat sealing including but not limited to a hot bar sealer, an impulse sealer; freehand welding whereby the hot air (or inert gas) is on the weld area and the tip of the weld rod at the same time; speed tip welding; extrusion welding, particularly, for joining materials over 6 mm thick; contact welding; hot plate welding; radio frequency welding; injection welding; ultrasonic welding; friction welding; spin welding; laser welding; transparent laser plastic welding; and solvent welding. These and other methods for welding plastics are well known in the art and one skilled in the art is able to employ any means to suit the needs of adhering or adjoining materials.

Any suitable method of sealing the cell chambers may be used. In some examples, methods of sealing include the employment of polymer adhesives, crimping, knotting and heat sealing. These sealing techniques are known in the art. In other embodiments, any suitable “dry” sealing method is used, as described in U.S. Pat. No. 5,738,673, which is incorporated by reference herein in its entirety. In such methods, a substantially non-porous fitting can be provided through which a therapeutic agent-containing solution (e.g., medium) is introduced into the chamber. Subsequent to filling, the device is sealed. Methods of sealing the devices are known in the art.

In another example, there is provided a method of closing a cell chamber or device that comprises wetting at least a portion of a permeable polymeric membrane of the device with a liquid and applying heat to at least a portion of a wetted thermoplastic polymer in association with the membrane to create a closure. Such a closure is referred to herein as a “wet seal.” In this “wet sealing” process, the thermoplastic polymer melts at a lower temperature than the polymeric membrane. Once melted, the thermoplastic polymer integrates with the polymeric membrane and flows along surfaces and into available interstices of the membrane. Through passageways become filled with the melted polymer, thereby blocking fluid communication in the polymeric membrane in the region of the closure. When the thermoplastic polymer cools below its melt temperature, a closure is formed in the device. The closure is cell-tight and often liquid-tight. The portion of the device having a closure formed with a wet seal delineates a cell-impermeable region of the device.

One example provides a method of closing a cell encapsulation device that comprises wetting a porous expanded polytetrafluoroethylene (ePTFE) membrane of the device with a liquid, and applying heat to a portion of the membrane in communication with a thermoplastic polymer, such as fluorinated ethylene propylene (FEP), to create a closure. The closure is formed by melting and fusing of the polymer to itself and the membrane in the presence of the liquid.

In one example there is a provided a method of closing a cell chamber or device that comprises applying sufficient heat to a portion of a permeable membrane in association with a thermoplastic polymer to melt and flow the thermoplastic polymer, followed by twisting the membrane/thermoplastic polymer combination in the region of the heating to form a closure. The membrane/thermoplastic polymer combination is also elongated while heating or twisting the materials. After heating, twisting, and elongation a separation region is formed and the membrane is cut in the separation region.

These and other “wet seal” methods of sealing are described in detail in U.S. Pat. No. 6,617,151.

### Ultrasonic Welding

Ultrasonic welding is a process that uses mechanical vibrations above the audible range. The vibrations, produced by a welding sonotrode or horn, as it is generally known, are used to soften or melt the thermoplastic material at the joint line.

### Heat Staking

Heat staking also known as thermoplastic staking is the process of joining two dissimilar materials together. In heat staking local heating and cooling is used to raise the temperature of plastic components and allow plastic reforming to be carried out.

### Welding of a NWF Layer to a Non-NWF Layer

A NWF layer and a non-NWF layer (e.g., a membrane) are, at the same time, cut and welded together. In some examples, a suction (or vacuum) is used to maintain a NWF layer and a non-NWF layer in close contact with each other when these layers are cut and welded to each other. A heat source is also required to melt the two layers together while they are being cut. These requirements result in high temperature precision cutting and welding of the NWF layer to the non-NWF layer; laser tack welding is one exemplary system. Laser tack welding is an example of one type of “vacuum welding” or “suction welding” system in which two layers are welded together and cut. Any other welding system that meets the requirements of precision cutting, melting, and suction (or vacuum) can be used to make the cell encapsulation devices disclosed herein. One of skill in the art would be able to design such a system.

### Laser Tack Welding of a Non-Woven Fabric to a Membrane

Laser tack welding does not compress the materials as seen with traditional lamination processes that use compression and heat, thus, resulting in less damage to the materials. The absence of compression on the membrane during the laser tack process ensures the functionality of the membrane and device has not been compromised.

A membrane can be made of, for example, an expanded polytetrafluoroethylene (ePTFE) or polytetrafluoroethylene (PTFE). The membrane is thin and porous and therefore very delicate, thus making it difficult to handle without damaging the membrane, for example, during manufacturing of the membrane or the cell encapsulation device.

Laser tack welding an ePTFE or PTFE membrane, for example, to a non-woven fabric or material (e.g., polyester) provides the membrane with increased mechanical integrity and reduces the likelihood of damage without impacting the functionality of the membrane. The increased stiffness of the laser welded materials improves device manufacturing by preventing the membrane from folding, wrinkling, or tearing during assembly of the membrane into the cell encapsulation device. The increased stiffness also improves component alignment during additional weld processes as the membrane will not wrinkle, fold, or tear when handled.

The laser tacking process is accomplished by placing the membrane (for example, made out of ePTFE or PTFE) directly on top of the non-woven fabric (polyester) supporting layer at a known distance from a CO₂ laser beam on an open grate surface. The order from the top is laser, membrane, NWF. Laser parameters are adjusted to ensure both adequate cutting and subsequent melting of materials are achieved. The materials are laser cut under suction (or vacuum) to ensure that they maintain direct contact throughout the cutting and tacking process and remove particulate that may be generated. Heat generated from the CO₂ laser cutting process melts the edges and interior portions of the ePTFE or PTFE and non-woven fabric layer creating a mechanical tack between the edges of both materials and creating a stiffer assembly.

Laser tack welding can be performed by developing a laser cutting method to cut both the membrane and a non-woven fabric together with enough laser power to melt the edges of these two layers together and to generate a weld. This weld keeps the two layers together and prevents the membrane from collapse post cutting. Traditional dye cutting methods can cut the layers of the device but do not seal the layers together at the same time.

For this purpose, a Universal Laser System VersaLaser (VLS) 3.5 equipped with a CO₂ laser can be used with the following exemplary program parameters:

| | | Raster | | | Laser | |
|---|---|---|---|---|---|---|
| | | Image | Vector | Laser | ARM | |
| Color | Mode | Density | Performance | Power | Speed | PPI |
| Black | Raster | 6 | N/A | 38% | 10% | 500 |
| Blue | Vector | N/A | +3 | 50% | 30% | 600 |
| Green | Vector | N/A | +3 | 50% | 30% | 600 |
| Red | Vector | N/A | +3 | 50% | 30% | 500 |

The non-woven fabric is placed on the laser grate and the membrane is placed on the NWF. The above parameters were set and cut per an exemplary design as illustrated in FIG. 5. Other laser systems known to one of skill in the art can be used.

### Flow Holes

As introduced above with reference to FIGS. 4-14, flow holes (or flow slits) can be cut into the NWF and non-NWF (e.g., membrane) layers and enable a film layer, located on both sides of the NWF/non-NWF (e.g., membrane) composite layer, when melted, to “flow” through the NWF/non-NWF (e.g., membrane) layer and melt into other film layers. Flow holes are weld-strengthening features of a cell encapsulation device. Flow holes enable the melting of film layers throughout the device resulting in a device with stronger welds.

The size of the flow holes can be determined by the size of the weld of a device. The flow holes desirably are contained within the boundaries of the weld. The size of the flow holes can be, for example, 0.2 mm+/−0.075, or about 0.125 mm, or about 0.01 mm to about 5.0 mm, or about 0.01 to about 9 mm, or about 0.001 mm to about 10 mm, 1 mm to 20 mm, or 0.001 mm to 20 mm. For example, the size of the flow holes can be about 0.1 mm to about 0.2 mm. The size of a flow hole can be, for example, any one of the following sizes in mm: 0.001 to 0.01, 0.01 to 0.02, 0.02 to 0.03, 0.03 to 0.04, 0.04 to 0.05, 0.05 to 0.06, 0.06 to 0.07, 0.07 to 0.08, 0.08 to 0.09, 0.09 to 0.1, 0.1 to 0.2, 0.2, to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 3.0, 3.0 to 4.0, 4.0 to 5.0, 5.0 to 6.0, 6.0 to 7.0, 7.0 to 8.0, 8.0 to 9.0, or 9.0 to 10.0. The sizes listed above, can be the diameter of a circular flow hole or the length or width of the flow hole, depending on the shape of the flow hole, for example a “slit” (see FIGS. 12-14, for example) or a rectangle.

A flow hole can be any size or shape. For example, a flow hole can be circular, non-circular (square, triangular, rectangular), or elongated (such as in the form of a slot or slit). Varying sizes and shapes of flow holes can be present within the area of the device weld.

The flow holes can be located along the perimeter of the device between the inner perimeter 312 and outer perimeter 316 as shown in FIG. 12.

The purpose of the flow holes is to enhance overall device weld strength by allowing a film, e.g., a thermo polycarbonate film (PCU), to flow through the membrane/NWF flow holes during the welding process (e.g., ultrasonic or heat staking) in order to “anchor” the NWF/membrane layer in between the adjacent layers of film. As shown in, for example, FIG. 5, two linear arrays of holes (flow holes 102) can be spaced approximately 1 mm apart in the area of the outer weld 121 (see e.g., FIG. 7). In some examples, these flow holes were added to a laser cut ePTFE membrane/NWF. The flow holes allow the multiple layers of film to flow and melt through the material layers of a device, creating a continuous piece of film that intercalates throughout the device layers. An enlarged area of the membrane/NWF is shown in inset “A” (see FIG. 6A) with flow holes 102.

In some examples, the flow holes 102 can have a diameter or width in a range of 0.001 mm to 20 mm. In some examples, the flow holes 102 can have a diameter of about (e.g., plus or minus a small machining tolerance) 0.20 mm.

Alternate flow hole array designs are shown, for example, in FIGS. 6B-6D and FIGS. 12-14. The shape of the holes can be different sizes, shapes, and the holes can be arranged in different patterns. The flow holes make the weld between the layers of the device stronger. The film that is melted acts as a “glue” to weld the layers of the device together.

An exemplary cell encapsulation device 100 comprising flow holes is shown in FIGS. 4-6A and 7. In some examples, this device consists of a stack-up of laser cut components that includes 6 layers of PCU film. Two of these film layers are adjacent to “sandwich” the membrane/NWF layer (108, 107) and create a seal that defines the lumen area (e.g., chambers 104) of the device when the device if fully assembled. In FIG. 7, for example, two lumens or chambers (chambers 104a, 104b) are shown. As described above, from the top of FIG. 4, the order of layers is: film, woven mesh, film, NWF layer laser tack welded to a membrane, film, device ports, then film, membrane laser tack welded to a NWF layer, film, woven mesh, and film. The device ports are made of, e.g., PCU tubing, each film is made of, e.g., PCU, the mesh is made of, e.g., polyethylene (PE), and the membrane is made of, e.g., ePTFE or PTFE.

In order to achieve optimal weld strength, direct contact in between the PCU film layers is desired to prevent delamination of the mesh components when subjected to continuous pressure. This direct contact is accomplished through melting the film layers during welding (e.g., ultrasonic or heat staking) of all of the other layers of the device (multiple film layers, mesh layers, etc.) with the NWF laser tack welded to the membrane. During the welding, the heat from the welding melts the film through the flow holes located along the perimeter of the NWF/membrane and connects or joins the melted film layers together. Flow holes allow the melted PCU film to flow in and through the membrane/NWF, connecting and melting into the other layers of film stacked through the device. The result of this increased melt strengthens the overall device weld area and produces higher Burst strength values as well as reduces risk of delamination caused by continuous pressure from inside the lumen.

### End Caps

One or more dimensions (e.g., a length and/or width) of a non-NWF (e.g., membrane) layer welded to a NWF layer, can be reduced by, e.g., 0.750 mm, relative to the overall length and width of its surrounding layers. A recessed membrane permits all the film layers to contact and bond to each other on the outer edges of the device creating a solid block of film referred to as an “end cap.”

A combination or composite non-NWF and NWF layer (e.g., a composite layer comprising layers 107, 108) can be reduced or recessed (or offset from the perimeter of the surrounding device layers) by any one of the following distances in mm: 0.001 to 0.01, 0.01 to 0.02, 0.02 to 0.03, 0.03 to 0.04, 0.04 to 0.05, 0.05 to 0.06, 0.06 to 0.07, 0.07 to 0.08, 0.08 to 0.09, 0.09 to 0.1, 0.1 to 0.2, 0.2, to 0.3, 0.3 to 0.4, 0.4 to 0.5, 0.5 to 0.6, 0.6 to 0.7, 0.7 to 0.8, 0.8 to 0.9, 0.9 to 1.0, 1.0 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, or 1.9 to 2.0. For example, about 0.5 to about 1.0, about 1.0 to about 2.0, about 2.0 to about 3.0, about 3.0 to about 4.0, or about 4.0 to about 5.0.

The amount that an end cap is recessed is dependent on the size of the device.

The end cap can be formed around the entire device. This enables the film layers to create a full seal all around the entire device. The end cap can also be around only a portion of the device, for example, along the two ends of the device located at opposing ends of the longest axis of the device.

As described above with reference to FIGS. 35A and 35B, FIG. 35A shows an SEM image of the Test Device 2 without the end cap feature, designated by the rectangle. FIG. 35B shows an SEM image of a Test Device 1 with an end cap feature designated by the rectangle.

### Possible Modifications Made to NWF/Non-NWF (Membrane) Layer

Design changes can be implemented by adding features to the NWF/membrane layer, or just the membrane layer, to promote better film-on-film contact and increase melting of the film layers during ultrasonic welding or heat staking. One of the main factors known to deter melting of a Bionate film ring is a PTFE membrane which has a significantly higher melting point than the film (327° C. (membrane) vs 180° C. (film)). One approach to addressing this issue is to add features to the membrane/NWF layer that allows for the flow of the film through the membrane/NWF layer.

In one example, three flow features were included in a multi-layered cell encapsulation device (e.g., device 100 of FIG. 4) to increase overall weld strength. During the process of laser tack welding the NWF layer to the membrane, the following flow features were formed: 1) flow holes were made to portions of the perimeter that would form the exterior (outer) weld, 2) an interior middle slit along the longest axis of the device was made (e.g., internal slit 119), which would ultimately form an interior weld, and 3) an “end cap”, wherein the NWF layer and the membrane were recessed or offset in relationship to their surrounding layers. In addition, the shape of the device was also cut during the laser tack welding process. Laser tack welding the NWF to the membrane resulted in increased stiffness of the laser tack welded materials, thus reducing the likelihood of damage to the membrane.

In the finished device, the NWF/membrane layer is “sandwiched” between two layers of film. The end cap is made by recessing the NWF/membrane layer around the perimeter, so that the two film layers that sandwich the NWF/membrane melt into each other (film-on-film contact) when the NWF/membrane layer is combined with the remaining layers and welded together.

Any of the three modifications or flow features described above, either together or separately, significantly increase the weld strength of a cell encapsulation device (perforated or not perforated). All three modifications or flow features are not required. During the welding (e.g., ultrasonic of heat staking) of the NWF/membrane layer with the remaining film layers of the device, the heat from the welding melts the film through the flow holes located along the perimeter of the NWF/membrane and also melts the film into the interior middle slit of the NWF/membrane, creating strong welds throughout the cell encapsulation device. The exterior welds are further strengthened by recessing the NWF/membrane layer to allow film-on-film melting during welding. The film (e.g., Bionate) has a melting temperature that is significantly lower than the other materials in the assembly such as the membrane). Combinations of modifications can be tested using the methods disclosed herein, such as burst testing, creep testing, or peel testing, for example, in addition to methods know to one of skill in the art. Any one of these three modifications or flow features can also be made only to the membrane, and then the membrane adhered to, attached to, or placed in contact with a NWF layer.

As set forth in the EXAMPLES section below: the following NWF/membrane modifications were implemented: 1) Added an internal slit (e.g., internal slit 119) feature that forms a middle weld (e.g., internal weld 126). Thus, when the NWF layer (e.g., NWF layer 107) is tack welded to the membrane layer (e.g., membrane layer 108) an internal slit is made in both layers. This internal slit is then filled with melted film when the layers are welded together by, for example, ultrasonic vibration using a sonotrode or heat staking. 2) A recessed NWF/membrane perimeter (e.g., recessed outer edge 114) to create an end cap feature along the weld perimeter (NWF/membrane edge is recessed from film edge to allow better film on film contact). 3) Addition of flow holes (e.g., flow holes 102) in the weld area—for example, 200 μm hole arrays were added to the NWF/membrane along the side weld areas (see FIGS. 7 and 19B). FIG. 7 shows the flow holes 102 in a linear arrangement on both sides of the NWF/membrane layer. A silhouette of the film layer 105 is shown for reference. In comparison, the device of FIG. 19A does not have flow holes.

In addition, two layers of film (see FIG. 4) can be added to the outside of the device, external to the mesh layer. The purpose of these two film ring layers (one on each side or wall of the device) is to enclose mesh fibers and maximize film-mesh intercalation.

### Method of Assembling a Cell Encapsulation Device

The following steps do not necessarily need to be in the order as listed below, unless changing the order would not result in the proper assembly of the cell encapsulation device. Further, not all of the steps listed below may be necessary, in some instances, or additional steps not listed can be used in assembling the device, in some instances. The parts of disclosed cell encapsulation devices as described herein are shown, for example, in FIGS. 1-21B.

### Exemplary Devices can be Made of:

1. 6 film layers
2. 2 mesh layers
3. A 1st NWF layer laser tack welded to a membrane
4. A 2nd NWF layer laser tack welded to a membrane
5. One or more pieces of tubing cut, for example, with a tube cutting fixture. A tube cutting fixture is a fixture with a razor blade to cut tubing to a desired dimension.

The membrane/NWF layer can have perforations or no perforations.

### A First Method of Welding Together Layers of a Device

Step 1. Create 1st NWF laser tack welded to a membrane:

Place membrane on top of non-woven fabric for laser tack welding. The laser is located above the membrane, and cuts through the membrane first and then the NWF layer.

The laser cuts flow holes, suture hole(s), center long hole(s) (internal slit(s) 119) that divide two chambers, and the perimeter in the shape of the device, in both the NWF and the membrane.

Laser cutting the membrane while it is on top of the NWF welds the membrane to the NWF at the following points: flow holes, suture hole(s), center long hole (s) (internal slit 119), and perimeter.

Repeat for 2nd NWF laser tack welded to a membrane.

For example, in FIGS. 4-11, each figure shows the NWF layer laser tack welded to the membrane layer. The “texture”/“appearance” shown by stippling, represents the NWF layer (e.g., NWF layer 107). The stippling is not to be confused with perforations as shown in several of the figures by reference number 110 or 510. Because the NWF is oriented towards the outside of the device, for example, as shown in FIG. 4, there is a difference in appearance of the top- and bottom-most layers of NWF tack welded to a membrane, as to depict this orientation.

Step 2. Using a laser, cut 6 film layers and 2 mesh layers individually in the shape of the device. Each of the 8 layers is cut individually to prevent any two layers from being laser tack welded together.

Step 3. The welding of the 6 film layers, 2 mesh layers, 1st NWF laser tack welded to a membrane, 2nd NWF laser tack welded to a membrane, and tubing, can be performed using, e.g., ultrasonic vibration and a sonotrode (i.e., ultrasonic welding) or by heat staking. A sonotrode is used to weld the layers together; the sonotrode is located above the stack of layers, the sonotrode presses on the layers from the top. In heat staking, an iron presses down on the layers that need to be welded together.

The order of stacking is: film, mesh, film, 1st NWF laser tack welded to a membrane, film, tube (device port), film, 2nd NWF laser tack welded to a membrane (with NWF facing outward), film, mesh, film, as shown in FIG. 4.

Step 4. For either welding technique (ultrasonic or heat staking), the 10 layers of the device can be placed in a cavity of a holder 600 (see e.g., FIG. 22) that is in the shape of the device. The tube (device port) is added to the cavity through an opening 616, and then the 10 layers are welded together. The ten layers are then flipped over in the cavity and welded again.

The center long hole (interior slit 119) in the NWF/membrane, in between two of the chambers, is then filled with the melted film from both layers of film that sandwich the NWF/membrane. The center portion of film melts into the center dividing cut (internal slit 119) of the NWF/membrane forming the barrier between the two chambers.

### A Second Method of Welding Together Layers of a Device

Step 1. Create 1st NWF laser tack welded to a membrane:

Place membrane on top of non-woven fabric for laser tack welding. The laser is located above the membrane, and cuts through the membrane first and then the NWF layer.

The laser cuts flow holes, suture hole(s), center long hole(s) (internal slit(s) 119) that divide two chambers, and the perimeter in the shape of the device, in both the NWF and the membrane.

Laser cutting the membrane while it is on top of the NWF, welds the membrane to the NWF at the following points: flow holes, suture hole(s), center long hole (s), and perimeter.

Repeat for 2nd NWF laser tack welded to a membrane.

For example, in FIGS. 4-11, each figure shows the NWF laser tack welded to the membrane. The “texture”/“appearance” shown by stippling, represents the NWF layer. The stippling is not to be confused with perforations as shown in several of the figures by reference number 110. Because the NWF is oriented towards the outside of the device, for example, as shown in FIG. 4, there is a difference in appearance of the top- and bottom-most layers of NWF tack welded to a membrane, as to depict this orientation.

Step 2. Using a laser, cut 6 film layers and 2 mesh layers individually in the shape of the device. Each of the 8 layers is cut individually to prevent any two layers from being laser tack welded together.

Step 3. A cell encapsulation device can be made in three parts. Unlike the first method described above, instead of 10 layers being stacked and welded together at once, three parts can be pre-welded and then the 3 pre-welded parts can be welded together.

Part 1: Film, mesh, film, 1st NWF laser tack welded to a membrane, film—these parts are welded together using, e.g., ultrasonic vibration or heat staking

Part 2: Film, mesh, film, 2nd NWF laser tack welded to a membrane, film—these parts are welded together using, e.g., ultrasonic vibration or heat staking

Part 3: Tubing

Step 4. The welding of the three parts can be performed using ultrasonic vibration and a sonotrode or by heat staking. A sonotrode is used to weld the layers together; the sonotrode is located above the stack of layers, the sonotrode presses on the layers from the top. In heat staking, an iron presses down on the layers that need to be welded together.

Step 5. Part 1, tubing (Part 3), and Part 2 are placed in a cavity of a holder 600 that is in the shape of the device (see e.g., FIG. 22). The tubes are added to the cavity through an opening 616, and then the three parts are welded together. The ten layers and tubing are then flipped over in the cavity and welded again.

The center long hole (internal slit 119) in the NWF/membrane, in between two of the chambers, is then filled with the melted film from both layers of film that sandwich the NWF/membrane. The center portion of film melts into the center dividing cut (internal slit 119) of the NWF/membrane forming the barrier between the two chambers.

### Creep Study

A creep study, also called “continuous lumen expansion test”, is used to test weld strength of a finished device. A certain pressure inside of the device lumen (or chamber) is applied for a certain period of time, e.g., four months, and the strength of the external weld along the perimeter and the suture holes is measured. A creep study or creep conditioning simulates the maximum pressure generated by a worst-case cell population (highly proliferative stem cells) within the device.

A creep study involves aging materials in the finished device under stress over time to represent the “useful life” of a device after being transplanted into a subject. A device is connected to an air supply with controlled pressure (psi) and submerged in water at a certain elevated temperature (e.g., above physiological temperatures) to simulate accelerated aging. This inflates the wetted device and stimulates stretching of the device over time under stress. For example, accelerated aging for 16 weeks at 55 degrees is equivalent to 3 years at 37 degrees in vivo. A “shelf life” is a time designated for a device to be stored prior to implantation that has been tested and verified by verification protocols.

### Peel Study

A peel study is a characterization study to determine visually how much delamination occurs between a layer of mesh and a layer of film that has been welded together (e.g., by ultrasonic vibration or heat staking). A segment of the weld is cut (e.g., 1 cm). A device is used to grab a layer of mesh at one point and all the other layers at another point. Then the mesh layer is pulled back to test how strong the bonds are that hold the mesh to the rest of the device.

The mesh layer is peeled from the remainder of the device using, for example, a Universal Materials Testing Machine (JD #D03) and NEXYGEN Plus software.

### Burst Study

A burst study is where a tubing from a pressure tester is connected to a device port of a finished device. A pressurized fluid having a predetermined pressure is applied to the device and the device is inflated until it bursts. A burst study tests weld integrity and strength.

A burst study can be performed after a creep study. In this instance, the device is dried and then it is submerged in 100% isopropyl alcohol. Then the device is connected to pressure tester and pressure is applied starting from 0 psi until the weld fails and the device bursts. A minimum 5 psi is required to pass a Burst Study.

### Device Materials

Useful biocompatible polymer devices comprise (a) a core which contains tissue or cells, and (b) a surrounding or peripheral region of biocompatible, semi-permeable membrane (jacket) which does not contain isolated cells.

The “semi-permeable” nature of the device membrane permits molecules produced by the cells (metabolites, nutrients and therapeutic substances) to diffuse from the device into the surrounding host tissue, but is sufficiently impermeable to protect the cells in the core from detrimental immunological attack by the host.

Cell permeable and impermeable membranes of have been described in the art including those previously referred to as TheraCyte cell encapsulation devices including. U.S. Pat. Nos. 6,773,458; 6,520,997; 6,156,305; 6,060,640; 5,964,804; 5,964,261; 5,882,354; 5,807,406; 5,800,529; 5,782,912; 5,741,330; 5,733,336; 5,713,888; 5,653,756; 5,593,440; 5,569,462; 5,549,675; 5,545,223; 5,453,278; 5,421,923; 5,344,454; 5,314,471; 5,324,518; 5,219,361; 5,100,392; and 5,011,494.

In one embodiment, the semi-permeable membrane is comprised of a biocompatible material including, but are not limited to, anisotropic materials, polysulfone (PSF), nanofiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, as well as hydroxylpropyl methyl cellulose (HPMC) membranes. These and substantially similar membrane types and components are manufactured by at least Gore®, Phillips Scientific®, Zeus®, Pall® and Dewal® to name a few.

Various polymers and polymer blends can be used to manufacture the device jacket, including, but not limited to, polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly (acrylonitrile/covinyl chloride), PTFE, as well as derivatives, copolymers and mixtures of the foregoing.

Biocompatible semi-permeable hollow fiber membranes, and methods of making them are disclosed in U.S. Pat. Nos. 5,284,761 and 5,158,881 (see also, WO 95/05452). In one embodiment, the device jacket is formed from a polyether sulfone hollow fiber, such as those described in U.S. Pat. Nos. 4,976,859 and 4,968,733.

### Device Loading Port

The phrases “device loading port” or “device loading tube” or “device port” or “device tube” or “device port tubing” means any tube that can be used to load therapeutic agents, such as cells, into a device. The device port (e.g., device port 109) connects directly with the interior of the device itself. If needed, the device can be primed with a medium or buffer prior to adding the therapeutic agents, by loading a medium or buffer into a device port that is connected to the device. The device port can be sealed using RF after the device is loaded.

More than one device port can be used to load a device. A device can have one chamber and two device ports. Alternatively, if two chambers exist in the device, each chamber will have one device port.

The device port can be directly connected to an aseptic connector that is connected to a reservoir of therapeutic agents, such as cells. Alternatively, the device port can be connected via a fitting to one or more tubes that are connected to an aseptic connector that is connected to a reservoir of therapeutic agents.

The device port can comprise, for example, polycarbonate urethane or a fluoropolymer.

In one embodiment, the device loading port (e.g., device port tubing 109) is made of any biocompatible flexible plastic tubing that responds particularly or selectively to radio frequency (RF) energy and internally heats and cools rapidly. For example, using a modest amount of pressure, the heated device loading port can be compressed shut to create a permanent seal in the tubing without the introduction of any additional sealing material (e.g., no sealant, or adhesive film) or damaging the device loading port. See, for example, FIG. 4, FIG. 1, FIG. 2, FIG. 3, and FIG. 18.

Device loading can be performed by either using a Hamilton syringe or the like plus a blunted appropriately sized gauge sterile needle (size will vary depending on the diameter of the port of the device) or the like, e.g., a 22-gauge needle. The needle is connected to the appropriate Hamilton syringe. The Hamilton syringe contains about 5-10 μL, 10-20 μL, 20-30 μL, 30-40 μL, 40-50 μL, 50-60 μL, 60-70 μL, 70-80 μL, 80-90 μL, 90-100 μL, 100-150 μL, 150-200 μL, 200-250 μL, 250-300 μL, 300-350 μL, 350-400 μL, or more than 400 μL of cell volume, which can reflect a therapeutically effective amount or dose of cells. The needle is then inserted through the at least one port of the device and into the lumen (or chamber) but without touching the walls of device. The entire contents of the syringe can be expelled slowly into the device, this occurs at the same time the needle is being withdrawn.

The device can be loaded via the device port using a pump (e.g., a peristaltic pump) or a pressure driven system using sterile air. For example, the pump, using external forces to move the solution could drive a cell solution from a compressible closed reservoir into the device. In another manufacturing format, sterile air could be applied to the cell solution to drive it into the device. Again, a therapeutically effective amount or dose of cells is used for these methods.

In another embodiment, the loading of the device is fully automated such that from the period the therapeutic agents (e.g., pancreatic endoderm cells) are thawed and cultured, until the cells (e.g., cell aggregates) are counted and loaded into the device, they are contained in closed and sterile environment.

### Therapeutic Agents

The embodiments disclosed herein describe loading therapeutic agents into an implantable device. The phrase “therapeutic agent” and “biologically active agent” are used interchangeably throughout the specification.

A therapeutic agent can be a cell or cells. As used herein, the term “cell”, “cells”, “cell aggregate” or “cell aggregates” or “cell aggregate suspension” or “cell suspension” may be used interchangeably throughout the specification depending on their context.

The term “cell” as used herein can refer to individual cells, cell lines, or cultures derived from such cells. A cell can be part of a tissue or organ. A “culture” or “cell culture” refers to a composition comprising isolated cells of the same or a different type. “Culture,” “population” or “cell population” as used herein can be and are used interchangeably and their meaning will be clear depending on the context. For example, the term “population” can be a cell culture of more than one cell having the same identifying characteristics or it can be a culture of more than one cell type having different identifying characteristics, e.g., a population in one context may be a sub-population in another context. The term “sub-population” refers to a subset of a cell culture or population when used to describe certain cell types within the cell culture or cell population.

As used herein, a “cell suspension” or equivalents thereof refers to a suspension of single cells, cell aggregates, a mixture of single cells and cell aggregates, or cell aggregates and/or clusters and/or spheres, that are contacted within a medium. Such cell suspensions are described in detail in U.S. application Ser. No. 12/264,760, entitled Stem cell Aggregate Suspension Compositions and Methods of Differentiation Thereof, filed on Nov. 8, 2008.

As used herein, the terms “cluster” and “clump” or “aggregate” can be used interchangeably, and generally refer to a group of cells that have not been dissociated into single cells and then aggregated to form clusters or have close cell-to-cell contact. The term “re-aggregated” as used herein refers to when clusters, clumps and/or aggregates are dissociated into smaller clusters, clumps and/or aggregates or single cells and then form new cell-to-cell contacts by re-aggregating into clusters, clumps and/or aggregates. This dissociation is typically manual in nature (such as using a Pasteur pipette), but other means of dissociation are contemplated. Aggregate suspension pluripotent or multipotent cell cultures are substantially as described in International Publications PCT/US2007/062755, titled COMPOSITIONS AND METHODS FOR CULTURING DIFFERENTIAL CELLS and PCT/US2008/082356, titled STEM CELL AGGREGATE SUSPENSION COMPOSITIONS AND METHODS OF DIFFERENTIATION THEREOF.

Although embodiments of the disclosure are described in the context of loading an implantable device with therapeutic agents, such as pancreatic progenitor cells and/or immature beta cells, one of ordinary skill in the art readily appreciates that the present disclosure is applicable for macro-encapsulation of any type of cells including cell aggregate suspensions, therapeutic agents, or mixtures thereof, including but not limited to thyroid cells, parathyroid cells, pancreatic cells, intestinal cells, thymus cells, hepatic cells, endocrine cells, skin cells, hematopoietic cells, bone marrow stem cells, renal cells, muscle cells, neural cells, stem cells, embryonic stem cells, lineage-restricted cells, progenitor cells, precursor cells, genetically engineered cells, tumor cells, and derivatives and combinations thereof for the treatment of one or more disease or disorder, including, but not limited to diabetes mellitus. Also contemplated are cells producing cell-based products such as proteins (e.g., hormones and/or other proteins deficient in human diseases and the like), antibodies, antibiotics, lymphokines and the like for therapeutic indications.

Embodiments of the disclosure are described in the context of loading an implantable cell encapsulation device with therapeutic agents, such as pancreatic progenitor cells, endocrine precursor cells, maturing beta cells, or mature beta cells, for the treatment of one or more disease or disorder, including, but not limited to diabetes mellitus. Diabetes can be type-1 diabetes, type-2 diabetes, or insulin-dependent diabetes.

The disclosure also contemplates using differentiable cells from any source within a mammal, for example, a human. For example, differentiable cells may be harvested from embryos, or any primordial germ layer therein, from placental or chorion tissue, or from more mature tissue such as adult stem cells including, but not limited to adipose, bone marrow, nervous tissue, mammary tissue, liver tissue, pancreas, epithelial, respiratory, gonadal and muscle tissue. In one embodiment, the differentiable cells are embryonic stem cells. In other another embodiment, the differentiable cells are adult stem cells. In still other embodiments, the stem cells are placental- or chorionic-derived stem cells.

The disclosure contemplates using differentiable cells from any mammal or animal capable of generating differentiable cells. The animals from which the differentiable cells are harvested may be vertebrate or invertebrate, mammalian or non-mammalian, human or non-human. Examples of animal sources include, but are not limited to, primates, rodents, canines, felines, equines, bovines and porcines.

The implantable device can comprise a therapeutic agent, a living cell, an endoderm-lineage cell, a definitive endoderm-lineage cell, a pancreatic progenitor cell, a pancreatic progenitor cell differentiated from a pluripotent cell, a progenitor cell differentiated from a stem cells, a human embryonic stem cell including those derived from methods now known or to be discovered in the future, including derivation using non-destruction of a human embryo or fetus, a cord blood stem cell, a fetal stem cell, an induced pluripotent stem cell, a reprogrammed cell, a parthenote cell, a gonadal germ cell, a mesenchymal cell, a hematopoietic stem cell, a pancreatic progenitor cell, a PDX-1 positive pancreatic progenitor cell, an endocrine precursor cell, an endocrine cell, an immature beta cell, or an immature islet cell.

The therapeutic agents can be endoderm lineage cells. General methods for production of endoderm lineage cells derived from hES cells are described in related U.S. applications as indicated herein, and D'Amour et al. 2005 Nat Biotechnol. 23:1534-41, published online Oct. 28, 2005; D'Amour et al. 2006 Nat Biotechnol. 24(11):1392-401, published online Oct. 19, 2006; Kroon et al. (2008) Nat Biotechnol. 26 (4):443-452, published online Feb. 20, 2008; Kelly et al. (2011) Nat. Biotechnol. 29(8):750-6, published online Jul. 31, 2011; Schulz et al. (2012) PLos One, 7(5): e37004, published online May 18, 2012, and Algunick et al. (2015) Stem Cells Transl. Med. Oct. 4 (10):1214-22, doi: 10.5966/sctm.2015-0079, Epub 2015 Aug. 24. D'Amour et al. describe a 5-step differentiation protocol: stage 1 (results in mostly definitive endoderm production), stage 2 (results in mostly PDX1-negative foregut endoderm production), stage 3 (results in mostly PDX1-positive foregut endoderm production), stage 4 (results in mostly pancreatic endoderm or pancreatic endocrine progenitor production) and stage 5 (results in mostly hormone expressing endocrine cell production).

The therapeutic agents can be pancreatic cell lineage cells. Methods for producing pancreatic cell lineages from human embryonic stem (hES) cells are substantially as described in U.S. Pat. No. 7,534,608, entitled METHODS OF PRODUCING PANCREATIC HORMONES, U.S. application Ser. No. 12/264,760, entitled STEM CELL AGGREGATE SUSPENSION COMPOSITIONS AND METHODS OF DIFFERENTIATION THEREOF, filed Oct. 4, 2008; U.S. application Ser. No. 11/773,944, entitled METHODS OF PRODUCING PANCREATIC HORMONES, filed Jul. 5, 2007; U.S. application Ser. No. 12/132,437, GROWTH FACTORS FOR PRODUCTION OF DEFINITIVE ENDODERM, filed Jun. 3, 2008; U.S. application Ser. No. 12/107,020, entitled METHODS FOR PURIFYING ENDODERM AND PANCREATIC ENDODERM CELLS DERIVED FROM HUMAN EMBRYONIC STEM CELLS, filed Apr. 8, 2008; U.S. application Ser. No. 11/875,057, entitled METHODS AND COMPOSITIONS FOR FEEDER-FREE PLURIPOTENT STEM CELL MEDIA CONTAINING HUMAN SERUM, filed Oct. 19, 2007; U.S. application Ser. No. 11/678,487, entitled COMPOSITIONS AND METHODS FOR CULTURING DIFFERENTIAL CELLS, filed Feb. 23, 2007; U.S. Pat. No. 7,432,104, entitled ALTERNATIVE COMPOSITIONS & METHODS FOR THE CULTURE OF STEM CELLS; Kroon et al. (2008) Nature Biotechnology 26(4): 443-452; D'Amour et al. 2005 Nat Biotechnol. 23:1534-41; D'Amour et al. 2006 Nat Biotechnol. 24(11):1392-401; McLean et al., 2007 Stem Cells 25:29-38.

The therapeutic agents can be mesendoderm or definitive endoderm-lineage type cells. Applicants have described in detail mesendoderm and definitive endoderm-lineage type cells in at least U.S. application Ser. No. 12/099,759, entitled METHODS OF PRODUCING PANCREATIC HORMONES, filed Apr. 8, 2008; Ser. No. 12/618,659, entitled ENCAPSULATION OF PANCREATIC LINEAGE CELLS DERIVED FROM HUMAN PLURIPOTENT STEM CELLS, filed Nov. 13, 2009; Ser. No. 14/106,330, entitled IN VITRO DIFFERENTIATION OF PLURIPOTENT STEM CELLS TO PANCREATIC ENDODERM CELLS (PEC) AND IMMATURE BETA CELLS, filed Dec. 12, 2013; Ser. No. 14/201,630, filed Mar. 7, 2014; and PCT/US2014/026529, IN VITRO DIFFERENTIATION OF PLURIPOTENT STEM CELLS TO PANCREATIC ENDODERM CELLS (PEC) AND ENDOCRINE CELLS, filed Mar. 13, 2014.

Other embodiments described herein relate to cell cultures of “PDX1-positive, dorsally-biased, foregut endoderm cells”, “PDX1-positive foregut endoderm cells”, or “PDX1-positive endoderm” or equivalents thereof. In some embodiments, the PDX1-positive foregut endoderm cells express PDX1, HNF6, SOX 9 and PROX 1 markers but do not substantially express NKX6.1, PTF1A, CPA, cMYC, SOX17, HNF1B or HNF4alpha. PDX1-positive foregut endoderm cell populations and methods of production thereof are also described in U.S. application Ser. No. 11/588,693, entitled PDX1-expressing dorsal and ventral foregut endoderm, filed Oct. 27, 2006. An “endocrine progenitor/precursor cell” as used herein refers to a multipotent cell of the definitive endodenn lineage that expresses at least a marker from the list consisting of neurogenin 3 (NEUROG3), PDX1, PTF1A, SOX9, NKX6.1, HNF1b, GATA4, HNF6, FOXA1, FOXA2, GATA6, MYT1, ISLET1, NEUROD, SNAIL2, MNX1, IA1, RFX6, PAX4, PAX6, NKX2.2, MAFA and MAFB which can further differentiate into cells of the endocrine system including, but not limited to, pancreatic islet hormone-expressing cells. Endocrine progenitor/precursor cells cannot differentiate into as many different cell, tissue and/or organ types as compared to less specifically differentiated definitive endoderm lineage cells, such as PDX1-positive pancreatic endoderm cells or definitive endoderm cells or mesendoderm cells. Endocrine progenitor/precursor cells are described in detail in at least Applicant's U.S. Pat. No. 8,129,182.

The therapeutic agents can be pancreatic endoderm cells. As used herein, the term “pancreatic endoderm cell” refers to a therapeutic cell source, according to embodiments of the disclosure, including but not limited to Applicants U.S. application Ser. No. 12/099,759, entitled METHODS OF PRODUCING PANCREATIC HORMONES, filed Apr. 8, 2008; Ser. No. 12/618,659, entitled ENCAPSULATION OF PANCREATIC LINEAGE CELLS DERIVED FROM HUMAN PLURIPOTENT STEM CELLS, filed Nov. 13, 2009; Ser. No. 14/106,330, entitled IN VITRO DIFFERENTIATION OF PLURIPOTENT STEM CELLS TO PANCREATIC ENDODERM CELLS (PEC) AND IMMATURE BETA CELLS, filed Dec. 12, 2013; Ser. No. 14/201,630, filed Mar. 7, 2014; and PCT/US2014/026529, IN VITRO DIFFERENTIATION OF PLURIPOTENT STEM CELLS TO PANCREATIC ENDODERM CELLS (PEC) AND ENDOCRINE CELLS, filed Mar. 13, 2014.

The terms “pancreatic endoderm,” “pancreatic epithelial,” “pancreatic epithelium” (all can be abbreviated “PE”), “pancreatic progenitor,” “PDX-1 positive pancreatic endoderm” or equivalents thereof, such as “pancreatic endoderm cells” (PEC), are all precursor or progenitor pancreatic cells. PEC as described herein is a progenitor cell population after stage 4 differentiation (about day 12-14) and includes at least two major distinct populations: i) pancreatic progenitor cells that express PDX1 and NKX6.1 but do not express CHGA (or CHGA negative, CHGA−), or “non-endocrine multipotent progenitor sub-populations (CHGA−)”, or “non-endocrine (CHGA−) sub-populations” or “non-endocrine (CHGA−) cells” or equivalents thereof; and ii) polyhormonal endocrine cells that express CHGA (CHGA positive, CHGA+), or “endocrine multipotent progenitor sub-populations (CHGA+)”, or “endocrine (CHGA+) sub-populations” or “endocrine (CHGA+) cells” or equivalents thereof. The PEC pancreatic progenitor subpopulation that express PDX1 and NKX6.1 but not CHGA is also referred to as “non-endocrine multipotent pancreatic progenitor sub-population (CHGA−)” or “non-endocrine progenitor sub-population,” “non-endocrine (CHGA−) sub-population,” “non-endocrine (CHGA−) sub-population,” “multipotent progenitor sub-population” and the like. The PEC polyhormonal endocrine cell subpopulation that expresses CHGA is also referred to as “cells committed to the endocrine lineage (CHGA+),” or endocrine cells” or “CHGA+ cells” and the like. Without being bound by theory, the cell population that expresses NKX6.1 but not CHGA is hypothesized to be the more active or therapeutic component of PEC, whereas the population of CHGA-positive polyhormonal endocrine cells is hypothesized to further differentiate and mature in vivo into glucagon-expressing islet cells. See Kelly et al. (2011) Cell-surface markers for the isolation of pancreatic cell types derived from human embryonic stem cells, Nat Biotechnol. 29(8):750-756, published online 31 Jul. 2011 and Schulz et al. (2012), A Scalable System for Production of Functional Pancreatic Progenitors from Human Embryonic Stem Cells, PLos One 7(5): 1-17, e37004.

Sometimes pancreatic endoderm cells are used without reference to PEC as described just above, but to refer to at least stages 3 and 4 type cells in general. The use and meaning will be clear from the context. Pancreatic endoderm derived from pluripotent stem cells, such as human embryonic stem cells and human induced pluripotent stem (IPS) cells, are distinguished from other endodermal lineage cell types based on differential or high levels of expression of markers selected from PDX1, NKX6.1, PTF1A, CPA1, cMYC, NGN3, PAX4, ARX and NKX2.2 markers, but do not substantially express genes which are hallmark of pancreatic endocrine cells, for example, CHGA, INS, GCG, GHRL, SST, MAFA, PCSK1 and GLUT1. Additionally, some “endocrine progenitor cells” expressing NGN3 can differentiate into other non-pancreatic structures (e.g., duodenum). Pancreatic endoderm or endocrine progenitor cell populations and methods thereof are also described in U.S. patent application Ser. No. 11/773,944, entitled Methods of producing pancreatic honnones, filed Jul. 5, 2007, and U.S. patent application Ser. No. 12/107,020, entitled METHODS FOR PURIFYING ENDODERM AND PANCREATIC ENDODERM CELLS DERIVED FORM HUMAN EMBRYONIC STEM CELLS, filed Apr. 21, 2008.

Other embodiments relate to cell cultures of “pancreatic endocrine precursor cells,” “pancreatic endocrine progenitor cells” or equivalents thereof. Pancreatic endocrine progenitor cells are multi potent and give rise to mature endocrine cells including alpha, beta, delta and PP cells. In some embodiments, the pancreatic endocrine progenitor cells express increased levels of NGN3, PAX4, ARX and NKX2.2 as compared to other non-endocrine progenitor cell types. Pancreatic progenitor cells also express low to no levels of INS, GCG, GHRL, SST, and PP.

The term “immature endocrine cell”, specifically an “immature beta-cell,” or equivalents thereof refer to a cell derived from any endocrine cell precursor including an endocrine progenitor/precursor cell, a pancreatic endoderm (PE) cell, a pancreatic foregut cell, a definitive endoderm cell, a mesendoderm cell or any earlier derived cell later described, that expresses at least a marker selected from the group consisting of INS, NKX6.1, PDX1, NEUROD, MNX1, NKX2.2, MAFA, PAX4, SNAIL2, FOXA1 or FOXA2. An immature beta cell described herein can express, INS, NKX6.1 and PDX1, or an immature beta cell can co-expresses INS and NKX6.1. The terms “immature endocrine cell,” “immature pancreatic hormone-expressing cell,” or “immature pancreatic islet” or equivalents thereof refer for example to at least a unipotent immature beta cell, or pre-beta cell, and do not include other immature cells, for example, the terms do not include an immature alpha (glucagon) cell, or an immature delta (somatostatin) cell, or an immature epsilon (ghrelin) cell, or an immature pancreatic polypeptide (PP).

The terms “endocrine cell” or “pancreatic islet hormone-expressing cell,” “pancreatic endocrine cell,” “pancreatic islet cell”, “pancreatic islets” or equivalents thereof refer to a cell, which can be polyhormonal or singly-hormonal. The cells can therefore express one or more pancreatic hormones, which have at least some of the functions of a human pancreatic islet cell. Pancreatic islet hormone-expressing cells can be mature or immature and are further differentiated or are further developmentally committed than an endocrine progenitor/precursor type cell from which they are derived.

As used herein the phrase “properly specified endocrine cells” or “stage 7 cultures” or “immature endocrine cells” including “immature beta cells” refers to endocrine cell populations made in vitro which are capable of functioning in vivo, e.g., immature beta cells when transplanted secrete insulin in response to blood glucose. Properly specified endocrine cells or stage 7 cultures may have additional characteristics including the following: When transplanted, properly specified endocrine cells may develop and mature into functional pancreatic islet cells. Properly specified endocrine cells may be enriched for endocrine cells (or depleted of non-endocrine cells). The properly specified endocrine cell population can be CHGA+. In one embodiment greater than about 50% of the cells in the properly specified endocrine cell population are CHGA+. In another embodiment greater than about 60% or 70% or 80% or 90% or 95% or 98% or 100% of the cells in the properly specified endocrine cell population are CHGA+. In one embodiment less than about 50% of the cells in the properly specified endocrine cell population are CHGA−. In another embodiment less than about 15% of the cells in the properly specified endocrine cell population are CHGA−. In one example less than about 10% or 5% or 3% or 2% or 1% or 0.5% or 0% of the cells in the properly specified endocrine cell population are CHGA−. Further, expression of certain markers may be suppressed in properly specified endocrine cells such as NGN3 expression during stage 3. Properly specified endocrine cells may have increased expression of NGN3 at stage 5. Properly specified endocrine cells may be singly-hormonal (e.g., insulin (INS) only, glucagon (GCG) only or somatostatin (SST) only). Properly specified endocrine cells may co-express other immature endocrine cell markers including NKX6.1 and PDX1. Properly specified endocrine cells may be both singly-hormonal and co-express other immature endocrine cell markers including NKX6.1 and PDX1. Properly specified endocrine cells may have more singly hormone expressing INS cells as a percentage of the total INS population. In another embodiment properly specified endocrine cells have at least 50% singly hormone expressing INS cells as a percentage of the total INS population. Properly specified endocrine cells may be CHGA+/INS+/NKX6.1+(triple positive). In one embodiment, greater than about 25% of the cells in the immature beta cell population are CHGA+/INS+/NKX6.1+(triple positive). In another embodiment, greater than about 30% or 40% or 50% or 60% or 70% or 80% or 90% or 95% 100% of the cells in the immature beta cell population are CHGA+/INS+/NKX6.1+(triple positive).

Still other examples relate to cell cultures of “pancreatic endocrine cells,” “pancreatic hormone secreting cells”, “pancreatic islet hormone-expressing cell,” or equivalents thereof refer to a cell, which has been derived from a pluripotent cell in vitro, e.g., alpha, beta, delta and/or PP cells or combinations thereof. The endocrine cells can be poly-hormonal or singly-hormonal, e.g., expressing insulin, glucagon, ghrelin, somatostatin and pancreatic polypeptide or combinations thereof. The endocrine cells can therefore express one or more pancreatic hormones, which have at least some of the functions of a human pancreatic islet cell. Pancreatic islet hormone-expressing cells can be mature or immature. Immature pancreatic islet hormone-expressing cells can be distinguished from mature pancreatic islet hormone-expressing cells based on the differential expression of certain markers, or based on their functional capabilities, e.g., glucose responsiveness in vitro or in vivo. Pancreatic endocrine cells also express low to no levels of NGN3, PAX 4, ARX and NKX2.2.

Alternatively, other examples relate to cell cultures of “PDX1-positive pancreatic endoderm tip cells,” or equivalents thereof. In some examples, the PDX1-positive pancreatic endoderm tip cells express increased levels of PDX1 and NKX6.1 similar to PDX1-positive pancreatic progenitor cells, but unlike PDX1-positive pancreatic progenitor cells, PDX1-positive pancreatic endoderm tip cells additionally express increased levels of PTF1A, CPA and cMYC. PDX1-positive pancreatic endoderm tip cells also express low to no levels of NGN3, PAX4, ARX and NKX2.2, INS, GCG, GHRL, SST, and PP.

The therapeutic agents can be derived from induced pluripotent stem cells. As used herein, the phrase “induced pluripotent stem cells,” or “iPS cells” or “iPSCs”, refer to any type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by inserting certain genes or gene products, referred to as reprogramming factors. See Takahashi et al., Cell 131:861-872 (2007); Wernig et al., Nature 448:318-324 (2007); Park et al., Nature 451:141-146 (2008). Induced pluripotent stem cells are substantially similar to natural human pluripotent stem cells, such as hES cells, in many respects including, the expression of certain stem cell genes and proteins, chromatin methylation patterns, doubling time, embryoid body formation, teratoma formation, viable chimera formation, and potency and differentiability. Human iPS cells provide a source of pluripotent stem cells without the associated use of embryos. Methods of making iPS cells are described in PCT/US2014/015156, filed Feb. 6, 2014, entitled, CELL COMPOSITIONS DERIVED FROM DEDIFFERENTIATED REPROGRAMMED CELLS.

In one aspect, a method for producing mature beta cells in vivo is provided. The method comprises making human definitive endoderm lineage cells derived from human pluripotent stem cells in vitro with at least a TGFβ superfamily member and/or at least a TGFβ superfamily member and a Wnt family member, preferably a TGFβ superfamily member and a Wnt family member, preferably Activin A, B or GDF-8, GDF-11 or GDF-15 and Wnt3a, preferably Actvin A and Wnt3a, preferably GDF-8 and Wnt3a. The method further comprises making PDX1-positive pancreatic endoderm cells from definitive endoderm cells with at least KGF, a BMP inhibitor and a retinoic acid (RA) or RA analog, and preferably with KGF, Noggin and RA. The method may further differentiate the PDX1-positive pancreatic endoderm cells into immature beta cells or MAFA expressing cells with a thyroid hormone and/or a TGFb-RI inhibitor, a BMP inhibitor, KGF, EGF, a thyroid hormone, and/or a Protein Kinase C activator; preferably with noggin, KGF and EGF, preferably additionally with T3 or T4 and ALK5 inhibitor or T3 or T4 alone or ALK5 inhibitor alone, or T3 or T4, ALK5 inhibitor and a PKC activator such as ILV, TPB and PdBu. Or preferably with noggin and ALK5i and implanting and maturing the PDXt-positive pancreatic endoderm cells or the MAFA immature beta cell populations into a mammalian host in vivo to produce a population of cells including insulin secreting cells capable of responding to blood glucose.

In one aspect, a unipotent human immature beta cell or PDX1-positive pancreatic endoderm cell that expresses INS and NKX6.1 and does not substantially express NGN3 is provided. In one example, the unipotent human immature beta cell is capable of maturing to a mature beta cell. In one example, the unipotent human immature beta cell further expresses MAFB in vitro or in vivo. In one example, the immature beta cells express INS, NKX6.1 and MAFA and do not substantially express NGN3.

In one aspect, pancreatic endoderm lineage cells expressing at least CHGA (or CHGA+) refer to endocrine cells; and pancreatic endoderm cells that do not express CHGA (or CHGA−) refer to non-endocrine cells. In another aspect, these endocrine and non-endocrine sub-populations may be multipotent progenitor/precursor sub-populations such as non-endocrine multipotent pancreatic progenitor sub-populations or endocrine multipotent pancreatic progenitor sub-populations; or they may be unipotent sub-populations such as immature endocrine cells, preferably immature beta cells, immature glucagon cells and the like.

In one aspect, more than 10% preferably more than 20%, 30%, 40% and more preferably more than 50%, 60%, 70%, 80%, 90%, 95%, 98% or 100% of the cells in the pancreatic endoderm or PDX1-positive pancreatic endoderm cell population (stage 4) are the non-endocrine (CHGA−) multipotent progenitor sub-population that give rise to mature insulin secreting cells and respond to glucose in vivo when implanted into a mammalian host.

One example provides a composition and method for differentiating pluripotent stem cells in vitro to substantially pancreatic endoderm cultures and further differentiating the pancreatic endoderm culture to endocrine or endocrine precursor cells in vitro. In one aspect, the endocrine precursor or endocrine cells express CHGA. In one aspect, the endocrine cells can produce insulin in vitro. In one aspect, the in vitro endocrine insulin secreting cells may produce insulin in response to glucose stimulation. In one aspect, more than 10% preferably more than 20%, 30%, 40% and more preferably more than 50%, 60%, 70%, 80%, 90%, 95%, 98% or 100% of the cells in the cell's population are endocrine cells.

Examples described herein provide for compositions and methods of differentiating pluripotent human stem cells in vitro to endocrine cells. In one aspect, the endocrine cells express CHGA. In one aspect, the endocrine cells can produce insulin in vitro. In one aspect, the endocrine cells are immature endocrine cells such as immature beta cells. In one aspect, the in vitro insulin producing cells may produce insulin in response to glucose stimulation.

One examples provides a method for producing insulin in vivo in a mammal, said method comprising: (a) loading a pancreatic endoderm cell or endocrine or endocrine precursor cell population into an implantable semi-permeable device; (b) implanting the device with the cell population into a mammalian host; and (c) maturing the cell population in said device in vivo wherein at least some of the endocrine cells are insulin secreting cells that produce insulin in response to glucose stimulation in vivo, thereby producing insulin in vivo to the mammal. In one aspect the endocrine cell is derived from a cell composition comprising PEC with a higher non-endocrine multipotent pancreatic progenitor sub-population (CHGA−). In another aspect, the endocrine cell is derived from a cell composition comprising PEC with a reduced endocrine sub-population (CHGA+). In another aspect, the endocrine cell is an immature endocrine cell, preferably an immature beta cell.

In one aspect the endocrine cells made in vitro from pluripotent stem cells express more PDX1 and NKX6.1 as compared to PDX-1 positive pancreatic endoderm populations, or the non-endocrine (CHGA−) subpopulations which are PDX1/NKX6.1 positive. In one aspect, the endocrine cells made in vitro from pluripotent stem cells express PDX1 and NKX6.1 relatively more than the PEC non-endocrine multipotent pancreatic progenitor sub-population (CHGA−). In one aspect, a Bone Morphogenic Protein (BMP) and a retinoic acid (RA) analog alone or in combination are added to the cell culture to obtain endocrine cells with increased expression of PDX1 and NKX6.1 as compared to the PEC non-endocrine multipotent progenitor sub-population (CHGA−). In one aspect BMP is selected from the group comprising BMP2, BMP5, BMP6, BMP7, BMP8 and BMP4 and more preferably BMP4. In one aspect the retinoic acid analog is selected from the group comprising all-trans retinoic acid and TTNPB (4-[(E)-2-(5,6,7,8-Tetrahydro-5,5,8,8-tetramethyl-2-naphthalenyl)-1-propenyl]benzoic acid Arotinoid acid), or 0.1-10 μM AM-580 (4-[(5,6,7,8-Tetrahydro-5,5,8,8-tetramethyl-2-naphthalenyl)carboxamido]benzoic acid) and more preferably TTNPB.

One example provides a method for differentiating pluripotent stem cells in vitro to endocrine and immature endocrine cells, preferably immature beta cells, comprising dissociating and re-associating the aggregates. In one aspect the dissociation and re-association occurs at stage 1, stage 2, stage 3, stage 4, stage 5, stage 6 or stage 7 or combinations thereof. In one aspect the definitive endoderm, PDX1-negative foregut endoderm, PDX1-positive foregut endoderm, PEC, and/or endocrine and endocrine progenitor/precursor cells are dissociated and re-associated. In one aspect, the stage 7 dissociated and re-aggregated cell aggregates consist of fewer non-endocrine (CHGA−) sub-populations as compared to endocrine (CHGA+) sub-populations. In one aspect, more than 10% preferably more than 20%, 30%, 40% and more preferably more than 50%, 60%, 70%, 80%, 90%, 95%, 98% or 100% of the cells in the cell population are endocrine (CHGA+) cells.

One example provides a method for differentiating pluripotent stem cells in vitro to endocrine cells by removing the endocrine cells made during stage 4 PEC production thereby enriching for non-endocrine multipotent pancreatic progenitor (CHGA−) sub-population which is PDX1+ and NKX6.1+.

In one example, PEC cultures enriched for the non-endocrine multipotent progenitor sub-population (CHGA−) are made by not adding a Noggin family member at stage 3 and/or stage 4. In one embodiment, PEC cultures which are relatively replete of cells committed to the endocrine lineage (CHGA+) are made by not adding a Noggin family member at stage 3 and/or stage 4. In one aspect the Noggin family member is a compound selected from the group comprising Noggin, Chordin, Follistatin, Folistatin-like proteins, Cerberus, Coco, Dan, Gremlin, Sclerostin, PRDC (protein related to Dan and Cerberus).

One example provides a method for maintaining endocrine cells in culture by culturing them in a media comprising exogenous high levels of glucose, wherein the exogenous glucose added is about 1 mM to 25 mM, about 1 mM to 20 mM, about 5 mM to 15 mM, about 5 mM to 10 mM, about 5 mM to 8 mM. In one aspect, the media is a DMEM, CMRL or RPMI based media.

One embodiment provides a method for differentiating pluripotent stem cells in vitro to endocrine cells with and without dissociating and re-associating the cell aggregates. In one aspect the non-dissociated or the dissociated and re-associated cell aggregates are cryopreserved or frozen at stage 6 and/or stage 7 without affecting the in vivo function of the endocrine cells. In one aspect, the cryopreserved endocrine cell cultures are thawed, cultured and, when transplanted, function in vivo.

Another example provides a culture system for differentiating pluripotent stem cells to endocrine cells, the culture system comprising of at least an agent capable of suppressing or inhibiting endocrine gene expression during early stages of differentiation and an agent capable of inducing endocrine gene expression during later stages of differentiation. In one aspect, an agent capable of suppressing or inhibiting endocrine gene expression is added to the culture system consisting of pancreatic PDX1 negative foregut cells. In one aspect, an agent capable of inducing endocrine gene expression is added to the culture system consisting of PDX1-positive pancreatic endoderm progenitors or PEC. In one aspect, an agent capable of suppressing or inhibiting endocrine gene expression is an agent that activates a TGFbeta receptor family, preferably it is Activin, preferably, it is high levels of Activin, followed by low levels of Activin. In one aspect, an agent capable of inducing endocrine gene expression is a gamma secretase inhibitor selected from a group consisting of N—[N-(3,5-Diflurophenacetyl-L-alanyl)]-S-phenylglycine t-Butyl Ester (DAPT), RO44929097, DAPT (N—[N-(3,5-Difluorophenacetyl-L-alanyl)]-S-phenylglycine t-Butyl Ester), 1-(S)-endo-N-(1,3,3)-Trimethylbicyclo[2.2.1]hept-2-yl)-4-fluorophenyl Sulfonamide, WPE-III31C, S-3-[N′-(3,5-difluorophenyl-alpha-hydroxyacetyl)-L-alanilyl]amino-2,3-dih-ydro-1-methyl-5-phenyl-1H-1,4-benzodiazepin-2-one, (N)—[(S)-2-hydroxy-3-methyl-butyryl]-1-(L-alaninyl)-(S)-1-amino-3-methyl-4,5,6,7-tetrahydro-2H-3-benzazepin-2-one, BMS-708163 (Avagacestat), BMS-708163, Semagacestat (LY450139), Semagacestat (LY450139), MK-0752, MK-0752, YO-01027, YO-01027 (Dibenzazepine, DBZ), LY-411575, LY-411575, or LY2811376. In one aspect, high levels of Activin is meant levels greater than 40 ng/mL, 50 ng/mL, and 75 ng/mL. In one aspect, high levels of Activin are used during stage 3 or prior to production of pancreatic foregut endoderm cells. In one aspect, low levels of Activin means less than 30 ng/mL, 20 ng/mL, 10 ng/mL and 5 ng/mL. In one aspect, low levels of Activin are used during stage 4 or for production of PEC. In one aspect, the endocrine gene that is inhibited or induced is NGN3. In another aspect, Activin A and Wnt3A are used alone or in combination to inhibit endocrine expression, preferably to inhibit NGN3 expression prior to production of pancreatic foregut endoderm cells, or preferably during stage 3. In one aspect, a gamma secretase inhibitor, preferably RO44929097 or DAPT, is used in the culture system to induce expression of endocrine gene expression after production of PEC, or preferably during stages 5, 6 and/or 7.

An in vitro cell culture comprising endocrine cells wherein at least 5% of said human cells express an endocrine marker selected from the group consisting of, insulin (INS), NK6 homeobox 1(NKX6.1), pancreatic and duodenal homeobox 1 (PDX1), transcription factor related locus 2 (NKX2.2), paired box 4 (PAX4), neurogenic differentiation 1 (NEUROD), forkhead box A1 (FOXA1), forkhead box A2 (FOXA2), snail family zinc finger 2 (SNAIL2), and musculoaponeurotic fibrosarcoma oncogene family A and B (MAFA and MAFB), and does not substantially express a marker selected from the group consisting of neurogenin 3 (NGN3), islet 1 (ISLI), hepatocyte nuclear factor 6 (HNF6), GATA binding protein 4 (GATA4), GATA binding protein 6 (GATA6), pancreas specific transcription factor 1a (PTF1A) and SRY (sex determining region Y)-9 (SOX9), wherein said endocrine cells are unipotent and can mature to pancreatic beta cells.

### Universal Donor Cells

The therapeutic agents can be universal donor cells or cells derived or differentiated from universal donor cells. “Hypoimmunogenic cells” or “universal donor cells” or “universal donor cell line” or “mutant cell” or equivalents thereof means a cell with reduced or eliminated expression of at least one HLA-Class I cell surface protein and at least one NK activating ligand. Such a cell is expected to be less prone to immune rejection or graft rejection by a subject into which such cells or graft are transplanted.

A universal donor cell line can be used to overcome graft rejection, in particular allogenic immune graft rejection in a cell-based transplantation therapy. In one embodiment, a cell derived from a human pluripotent stem cell, such as a pancreatic cell, is provided that lack some or all classic HLA-Class I cell surface protein expression and NK activating ligand expression. In one example, there is provided a method of preventing cellular graft rejection by transplanting into a mammalian subject pancreatic cells where the function of at least one MHC gene, such as beta-2-microgobulin (B2M), and at least one NK activating ligand, such as Intercellular Adhesion Molecule 1 (ICAM-1), has been disrupted, deleted, modified, or inhibited. Disruption, deletion, modification, or inhibition of B2M, results in deficiency in all of HLA class I surface expression and function.

A universal donor cell line can be an in vitro cell population comprising pancreatic lineage cells, wherein the function of at least one major histocompatibility complex (MHC)-Class I gene and at least one Natural killer (NK) cell activating ligand is disrupted or inhibited in the pancreatic lineage cells. In one embodiment, the MHC-Class I gene of the pancreatic lineage cells encodes beta-2 microglobulin (B2M) or a human leukocyte antigen (HLA)-ABC cell surface protein. In other embodiments, the NK cell activating ligand of the pancreatic lineage cells is intercellular adhesion molecule (ICAM)1, cluster of differentiation (CD)58, CD155, poliovirus receptor (PVR), carcinoembryonic antigen related cell adhesion molecule (CEACAM)1, cell adhesion molecule (CADM)1, major histocompatibility class I related chain protein (MIC)A, MICB, or a combination thereof. In other embodiments, a combination of NK cell activating ligands is disrupted or inhibited in the pancreatic lineage cells, such as a) CD58 and ICAM1; b) CD58, ICAM1, and CD155; c) CD58 and CADM1; d) CD58 and CD155; e) CD58, ICAM1, CD155, and CADM1; or f) ICAM1, CADM1, and CD155.

Some aspects of the present disclosure relate to cell cultures or cell populations comprising from at least about 5% of a certain cell type as disclosed herein (e.g., pancreatic endoderm cell or a universal donor cell line) to at least about 95% of the cell type. In some examples the cell cultures or cell populations comprise mammalian cells. In preferred examples, the cell cultures or cell populations comprise human cells. For example, certain specific examples relate to cell cultures comprising human cells, wherein from at least about 5% to at least about 95% of the human cells are a certain cell type as disclosed herein (e.g., pancreatic endoderm cell or a universal donor cell line). Other examples of the present invention relate to cell cultures comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or greater than 90% of the human cells are a certain cell type as disclosed herein (e.g. pancreatic endoderm cell or a universal donor cell line).

### Sterilization

Each part of the device, or the entire device can be sterilized by ethylene oxide, electron beam, radiation (e.g., ionizing radiation, gamma irradiation, electron beam irradiation), dry heat exposure or high-temperature moisture (e.g., steam (wet heat exposure)), autoclave, or chemical exposure (e.g., ethylene oxide exposure).

### Adhesives

Conventional lamination and sealing techniques employ, for example, use of a spray adhesive, roll coating, knife over roll coating, wire rod coating, or gravure coating. Suitable adhesives include solvent based, water based, or solvent-less adhesives, including acrylic adhesives, epoxy cured polyester urethanes, moisture cured polyester urethanes, and isocyanate terminated polyester adhesives.

### EXAMPLES

### Example 1: Test Device 2 (a Cell Encapsulation Device with No Flow Holes, No End Caps, No Internal Slit, and Four Film Layers) Failed Creep Testing

As introduced above, Test Device 2 was constructed in accordance with device 500 shown in FIG. 18. However, Test Device 2 was not perforated (no perforations 510). A non-perforated version of the device 500 shown in FIG. 18 was used because air will escape through the perforations during creep and burst testing. A non-perforated version of the device of FIG. 18 was used in order to assess device and weld integrity.

In Test Device 2 there are no flow holes or end caps, and there are four film layers (e.g., film layers 505). There is no internal slit in the middle of the NWF layer (e.g., NWF layer 507) heat laminated to the membrane (e.g., membrane layer 508). The outer most layer is a non-woven fiber. There is also a layer of mesh that provides support/stiffness to the device. The NWF supports anchoring of the device to host cell tissue upon transplantation of the device, through its non-woven design.

Test Device 2 was tested to see if it met the device and weld integrity requirements after creep conditioning. Testing was performed in vitro under aqueous conditions at physiological temperatures for a minimum duration of 16 weeks. Device integrity of Test Device 2 articles is determined by satisfying the visual inspection criteria at 4-week intervals. Test Device 2 meets the weld integrity requirement by satisfying the minimum burst pressure criteria at the final 16-week time point. A total of 59 Test Device 2 articles were attached to a Y-connector, pouched, and sterilized twice via ethylene oxide prior to Creep conditioning.

TABLE 1
Design Specification for Creep Testing

| Design ID and Description | Device Output | Device Specification |
|---|---|---|
| 6.16 - Creep: device must | 6.13 - Weld Burst | Device weld must have a |
| pass the following | | burst pressure ≥5.0 psi |
| specifications after being | 6.19 - Device Integrity | Device mesh and weld must |
| Creep conditioned | | pass visual inspection for |
| | | breaks, tears, and |
| | | delamination |

Test Device 2 did not successfully meet the device and weld integrity requirements after creep conditioning, creep conditioning was stopped prematurely at 2 weeks due to 9 test articles containing mesh delamination, one test article having bubbles coming from the device, and several devices bursting. FIG. 33 shows an image of a broken weld in Test Device 2. FIG. 34A and FIG. 34B show two different images of mesh delamination that occurred in Test Device 2.

The Creep Verification Visual Inspection and Burst Pressure Testing results are summarized in Table 2 below. Since the Test Device 2 articles were removed after 2 weeks of Creep conditioning, the test articles were not visually inspected or tested for Burst at any time point.

TABLE 2
Creep Verification Test Results

| | | Sample | | | |
|---|---|---|---|---|---|
| Test | Acceptance Criteria | Size | # of Passed | # of Failed | # Not Tested |
| Device | Device mesh and weld | 59 | 0 | 9 | 50 |
| Integrity | must pass visual | | | | |
| | inspection for breaks, | | | | |
| | tears, and delamination | | | | |
| Weld Burst | Device weld must have | 59 | N/A | N/A | 59 |
| | burst pressure ≥5.0 psi | | | | |

### Table 2. Creep Verification Test Results

To determine the root cause of the mesh delamination and burst/broken welds of the Test Device 2 articles, an Ishikawa analysis was performed. Investigation of the identified possible root causes was conducted and consisted of burst testing, peel testing, and scanning electron microscopy (SEM) imaging to compare weld and bond strengths of several device configurations including Test Device 2 and Test Device 1 (described below). The analysis determined that the root cause for the mesh delamination and broken welds was a combination of poor weld strength and lack of intercalation of the film layers of the device with the other non-film layers of the device, e.g., a mesh layer.

To further characterize the root causes, the location of the device failure was determined. The general failure location was determined to be on the top-right side, distal from the port. Seven of the nine failed devices were available for delamination location measurements. Measurements were taken from the device port end of the device to the center of the delamination using calipers. See FIG. 36 and Table 3 below for delamination and location measurements.

TABLE 3
Delamination Location Measurements

| | | Location of | |
|---|---|---|---|
| Sample Number | Device Number | Delamination (mm) | % of Device Height |
| 1 | B3 | 35.80 | 41.48 |
| 2 | G7 | 44.34 | 51.38 |
| 3 | A8 | 40.07 | 46.43 |
| 4 | E8 | 38.22 | 44.29 |
| 5 | H3 | 33.37 | 38.67 |
| 6 | C5 | 29.41 | 34.08 |
| 7 | E5 | 34.43 | 39.90 |
| | | Mean: | 42.75 |
| | | Standard Deviation: | 6.490 |

### Table 3. Delamination Location Measurements

The delamination location was determined to be slightly less than halfway up the side of the device weld as shown in FIG. 36. It was concluded that this location of the device had a weaker weld that the rest of the device.

The peel strength was determined for 12 Test Device 2 articles pre- and post-creep conditioning. Several locations of the device were peel tested. FIG. 26 shows the peel strengths at each location. The locations tested were differentiated by the top and bottom (T or B) sides of the device, left and right (L or R), and distal or proximal to the device port (D or P). “Pre-Creep” is a device prior to a Creep study, “Creep” is a device after a Creep study has been conducted.

The conclusion of this study was that the weaker weld on the right-hand side of the middle of the device, can be supported by the peel strength data as the data correlates with the delamination location occurring on the right side in the middle of the device.

### Example 2: Test Device 1 with End Caps has Higher Peel Strength Values Resulting in a Tighter Weld than a Device without End Caps, Due to Film-on-Film Melting

As introduced above, Test Device 1 was constructed in accordance with device 400 shown in FIGS. 15-17B. The end caps were formed around the entire periphery of the device. However, Test Device 1 does not include perforations. PD Test device 1 has perforations. A non-perforated version of PD Test device 1 was used because air will escape through the perforations during Creep and Burst testing. A non-perforated version of the device must be used in order to assess device and weld integrity.

Test Device 1 consists of: a recessed outer edge (e.g., recessed outer edge 414), which are recessed membranes (e.g., membranes 408) between two film ring (e.g., film ring 405) layers, six layers of film rings, and an internal slit (e.g., internal slit 419) in the middle of the membrane.

### End Caps

Reviewing the SEM images of the Test Device 2 articles and the Test Device 1 articles, the membrane component of the Test Device 1 articles was reduced by approximately 0.750 mm relative to the overall device length and width, referred to as a recessed membrane (with end cap). A recessed membrane permits all the film layers to contact and bond to each other on the outer edges of the device creating a solid block of film referred to as an ‘end cap.” FIG. 35A shows an SEM image of the Test Device 2 without the end cap feature, designated by the rectangle. FIG. 35B shows an SEM image of a Test Device 1 with an end cap feature, designated by the rectangle.

To quantify the end cap feature, Test Device 1 articles were tested for peel strength by pulling the membrane and mesh layer from the remainder of the device. Two data points were taken during the peel testing: the first peak and second peak. The first peak shows the maximum peel strength of the membrane-to-film bond, and the second peak shows the maximum peel strength of the film-to-film bond, or end cap feature. FIG. 25 shows the peel strength values of the Test Device 1 articles. From the data, the end cap feature of the Test Device 1 articles have stronger peel strength than the membrane-to-film bond in the Test Device 2 articles (FIG. 27).

### Example 3: Test Device 1 and Test Device 2 Showed Similar Weld Strength when Burst Tested

Burst data of the Test Device 1 articles and the Test Device 2 articles is shown in FIG. 24. FIG. 24 shows that the weld strength of the Test Device 2 articles was comparable to that of the Test Device 1 articles despite having removed two layers of film (See FIG. 15 for Test Device 1 and FIG. 18 for Test Device 2). Based on these results it was surprising that the Test Device 2 articles failed creep testing (See EXAMPLE 1).

Some of the devices with mesh delamination were still able to hold pressure inside the lumen, which meant that the outer film layers were not melting to the same degree as the inner film layers in the stack-up. The rapid inflation that occurred during Burst testing may have resulted in the test lacking the sensitivity to detect partial failures occurring in the outer layers of the stack-up. Although burst testing is an important indicator of the degree of melting between the inner film layers, it became necessary to develop additional test methods that could measure the force needed to cause mesh delamination.

### Example 4: Test Device 2 had Stronger Weld Strength than Test Device 1 when Peel Tested

As mentioned in EXAMPLE 3 above, there was a need to develop and implement a second test, in addition to a Burst test, that would provide a more accurate estimate of the weld strength related to mesh delamination. Peel strength testing was conducted.

A second test was developed where 15 mm width segments were cut from side and middle weld locations of the Test Device 1 articles and Test Device 2 articles, and subsequently Peel tested to failure. Test samples were placed in the tensile tester isolating the mesh in one of the clamps and pulling it from the rest of the weld layers in order to more accurately measure peel strength needed for mesh delamination.

Initial peel data comparing the Test Device 1 articles and Test Device 2 articles, revealed surprising and significant differences in Peel strength between the two device configurations, as shown in FIG. 23 (as introduced above). Test Device 1 articles showed close to a 4-fold increase in weld Peel strength as compared to Test Device 2 articles, along the perimeter weld of the device. In addition, Test Device 1 articles showed greater than a 2-fold increase in weld Peel strength as compared to Test Device 2 along the middle (or interior) weld of the device.

### Example 5: The Presence of Flow Holes, End Caps, and an Internal Weld in Test Device 3 Articles Results in Stronger Welds than Test Device 2 Articles that Lack Flow Holes, End Caps, and an Internal Weld

As introduced above, Test Device 3 was constructed in accordance with device 100 shown in FIGS. 4-6A and 7-8. However, Test Device 3 does not include perforations (e.g., perforations 110).

In Test Device 3 there are flow holes, end caps, and there are six film layers (e.g., film rings 105). There is an internal slit in the middle of the NWF layer laser tack welded to the membrane layer, as described above with reference to FIGS. 4, 5, 7, and 8.

FIG. 27 is a comparison of weld Peel data of Test Device 2 articles versus Test Device 3 articles. FIG. 27 shows that Test Device 3 articles had stronger welds than the Test Device 2 articles due to the presence of flow holes, end caps, and an internal slit in the NWF layer laser tack welded to the membrane. The presence of two additional film rings in the Test Device 3 articles also added to the strength of the welds. The Test Device 2 articles do not have flow holes, end caps, and an internal slit in the NWF layer heat laminated to the membrane. Test Device 2 articles only have four film rings.

### Example 6: Device Component Design Modifications

Based on the above studies, design changes were implemented by adding features to the NWF/membrane layer, to promote better film-on-film contact and increase melting of the film layers during ultrasonic welding. One of the main factors known to deter melting of a Bionate film ring is the PTFE membrane which has a significantly higher melting point than the film (327° C. (membrane) vs 180° C. (film)). One approach to addressing this issue was to add features to the membrane/NWF layer that allows for the flow of the film through the membrane/NWF layer.

The following NWF/membrane modifications were implemented (for example, as shown in device 100): 1) Added an internal slit feature (e.g., internal slit 119) that forms a middle weld. Thus, when the NWF layer is tack welded to the membrane, an internal slit is made in both layers. This internal slit is then filled with melted film when the layers are welded together by, for example, ultrasonic vibration using a sonotrode or heat staking. 2) A recessed membrane perimeter (e.g., recessed outer edge 114) to create an end cap feature along the weld perimeter (membrane edge is recessed from film edge to allow better film on film contact). 3) Addition of flow holes (e.g., flow holes 102) in the weld area—for example, 200 μm hole arrays were added to the membrane along the side weld areas. For example, FIG. 5 and FIG. 7 shows flow holes 102 in a linear arrangement on both sides of the NWF/membrane layer. A silhouette of the film layer is shown for reference in FIG. 7.

The second major change involved adding two layers of film layers on the exterior of the device (one on each side) (see FIG. 4, for example). The purpose of these two additional film ring layers was to enclose mesh fibers and maximize film-mesh intercalation, as shown in the SEM images of FIGS. 32A and 32C.

### Example 7: Test Device 3 Articles with Flow Holes, End Caps, and an Internal Weld Passed Creep Testing

Creep verification of Test Device 3 articles was conducted as described in EXAMPLE 1, with minor deviations, up to the 12-week time point. At the 16-week time point, visual inspection and Burst pressure testing were executed.

59 Test Device 3 articles underwent creep conditioning. Daily verification of the pressure, flow rate, and water bath temperature for the creep test system was recorded (data not shown). Visual inspection data was recorded for the inspection at the 4-week, 8-week, 12-week, and 16-week time points. Burst pressure testing data was recorded (data not shown).

TABLE 4
Design Specification for Creep Testing

| Design ID and Description | Device Output | Device Specification |
|---|---|---|
| 6.16 - Creep: device must | 6.13 - Weld Burst | Device weld must have a |
| pass the following | | burst pressure ≥5.0 psi |
| specifications after being | 6.19 - Device Integrity | Device mesh and weld must |
| Creep conditioned | | pass visual inspection for |
| | | breaks, tears, and |
| | | delamination |

### Table 4. Design Specification for Creep Testing

The creep verification visual inspection and Burst pressure testing results are summarized in Table 5 below.

TABLE 5
Creep Verification Test Results

| | | | 4 weeks | | 8 weeks | | 12 weeks | | 16 weeks | |
|---|---|---|---|---|---|---|---|---|---|---|
| | Acceptance | Sample | # of | # of | # of | # of | # of | # of | # of | # of |
| Test | Criteria | Size | passed | failed | passed | failed | passed | failed | passed | failed |
| Device | Device | 59 | 59 | 0 | 59 | 0 | 59 | 0 | 59 | 0 |
| integrity | Mesh and | | | | | | | | | |
| | Weld must | | | | | | | | | |
| | pass visual | | | | | | | | | |
| | inspection | | | | | | | | | |
| | for breaks, | | | | | | | | | |
| | tears, and | | | | | | | | | |
| | delamination | | | | | | | | | |
| Weld | Device weld | 59 | N/A | N/A | N/A | N/A | N/A | N/A | 59 | 0 |
| Burst | must have | | | | | | | | | |
| | burst | | | | | | | | | |
| | pressure ≥5.0 | | | | | | | | | |
| | psi | | | | | | | | | |

The Test Device 3 articles met the device and weld integrity with 95% confidence and 95% reliability after 16 total weeks of creep conditioning. Following creep conditioning, the Test Device 3 articles, were visually inspected and underwent burst pressure testing.

TABLE 6
Design Specification for Creep Testing Results

| 6.16 Creep | | | |
|---|---|---|---|
| Specification | Specification | Acceptance Criteria | Result |
| Device must pass the | Device Integrity | Device Mesh and | PASS |
| following | | Weld must pass | |
| specifications after | | visual inspection for | |
| being conditioned | | breaks, tears, and | |
| | | delamination | |
| | Weld Burst | Device weld must | PASS |
| | | have burst | |
| | | pressure ≥5.0 psi | |

### REFERENCES

Various aspects of the disclosure are described herein, but still others not described in detail but referred to can be found in Applicant's U.S. patent application Ser. No. 10/486,408, entitled METHODS FOR CULTURE OF HESC ON FEEDER CELLS, filed Aug. 6, 2002; Ser. No. 11/021,618, entitled DEFINITIVE ENDODERM, filed Dec. 23, 2004; Ser. No. 11/115,868, entitled PDX1 EXPRESSING ENDODERM, filed Apr. 26, 2005; Ser. No. 11/165,305, entitled METHODS FOR IDENTIFYING FACTORS FOR DIFFERENTIATING DEFINITIVE ENDODERM, filed Jun. 23, 2005; Ser. No. 11/573,662, entitled METHODS FOR INCREASING DEFINITIVE ENDODERM DIFFERENTIATION OF PLURIPOTENT HUMAN EMBRYONIC STEM CELLS WITH PI-3 KINASE INHIBITORS, filed Aug. 15, 2005; 12/729,084 entitled PDX1-EXPRESSING DORSAL AND VENTRAL FOREGUT ENDODERM, filed Oct. 27, 2005; Ser. No. 12/093,590, entitled MARKERS OF DEFINITIVE ENDODERM, filed Nov. 14, 2005; Ser. No. 11/993,399, entitled EMBRYONIC STEM CELL CULTURE COMPOSITIONS AND METHODS OF USE THEREOF, filed Jun. 20, 2006; Ser. No. 11/588,693, entitled PDX1-EXPRESSING DORSAL AND VENTRAL FOREGUT ENDODERM, filed Oct. 27, 2006; Ser. No. 11/681,687, entitled ENDOCRINE PROGENITOR/PRECURSOR CELLS, PANCREATIC HORMONE-EXPRESSING CELLS AND METHODS OF PRODUCTION, filed Mar. 2, 2007; Ser. No. 11/807,223, entitled METHODS FOR CULTURE AND PRODUCTION OF SINGLE CELL POPULATIONS OF HESC, filed May 24, 2007; Ser. No. 11/773,944, entitled METHODS OF PRODUCING PANCREATIC HORMONES, filed Jul. 5, 2007; Ser. No. 11/860,494, entitled METHODS FOR INCREASING DEFINITIVE ENDODERM PRODUCTION, filed Sep. 24, 2007; Ser. No. 12/099,759, entitled METHODS OF PRODUCING PANCREATIC HORMONES, filed Apr. 8, 2008; Ser. No. 12/107,020, entitled METHODS FOR PURIFYING ENDODERM AND PANCREATIC ENDODERM CELLS DERIVED FORM HUMAN EMBRYONIC STEM CELLS, filed Apr. 21, 2008; Ser. No. 12/618,659, entitled ENCAPSULATION OF PANCREATIC LINEAGE CELLS DERIVED FROM HUMAN PLURIPOTENT STEM CELLS, filed Nov. 13, 2009; Ser. No. 12/765,714 and Ser. No. 13/761,078, both entitled CELL COMPOSITIONS FROM DEDIFFERENTIATED REPROGRAMMED CELLS, filed Apr. 22, 2010 and Feb. 6, 2013; Ser. No. 11/838,054, entitled COMPOSITIONS AND METHODS USEFUL FOR CULTURING DIFFERENTIABLE CELLS, filed Aug. 13, 2007; Ser. No. 12/264,760, entitled STEM CELL AGGREGATE SUSPENSION COMPOSITIONS AND METHODS OF DIFFERENTIATION THEREOF, filed Nov. 4, 2008; 13/259,15, entitled SMALL MOLECULES SUPPORTING PLURIPOTENT CELL GROWTH, filed Apr. 27, 2010; PCT/US11/25628, entitled LOADING SYSTEM FOR AN ENCAPSULATION DEVICE, filed Feb. 21, 2011; Ser. No. 13/992,931, entitled AGENTS AND METHODS FOR INHIBITING PLURIPOTENT STEM CELLS, filed Dec. 28, 2010; and U.S. Design application Numbers: 29/408,366 filed Dec. 12, 2011; Ser. No. 29/408,368 filed Dec. 12, 2011; Ser. No. 29/423,365 filed May 31, 2012; and Ser. No. 29/447,944 filed Mar. 13, 2013; and U.S. Provisional Application No.: 61/774,443, entitled SEMIPERMEABLE MACRO IMPLANTABLE CELLULAR ENCAPSULATION DEVICES, filed Mar. 7, 2013; 61/775,480, entitled CRYOPRESERVATION, HIBERNATION AND ROOM TEMPERATURE STORAGE OF ENCAPSULATED PANCREATIC ENDODERM CELL AGGREGATES, filed Mar. 8, 2013; Ser. No. 14/106,330, entitled IN VITRO DIFFERENTIATION OF PLURIPOTENT STEM CELLS TO PANCREATIC ENDODERM CELLS (PEC) AND ENDOCRINE CELLS, filed Dec. 13, 2013; PCT/US2014/022065, entitled CRYOPRESERVATION, HIBERNATION AND ROOM TEMPERATURE STORAGE OF ENCAPSULATED PANCREATIC ENDODERM CELL, filed Mar. 7, 2014; PCT/US2014/022109, SEMIPERMEABLE MACRO IMPLANTABLE CELLULAR ENCAPSULATION DEVICES, filed Mar. 7, 2014; Ser. No. 14/201,630, filed Mar. 7, 2014; and PCT/US2014/026529, IN VITRO DIFFERENTIATION OF PLURIPOTENT STEM CELLS TO PANCREATIC ENDODERM CELLS (PEC) AND ENDOCRINE CELLS, filed Mar. 13, 2014.

Applicants have described both a renewable cell source and macro-encapsulation drug delivery system suitable for at least the purpose of pancreatic progenitor cell delivery for production of insulin in vivo in response to glucose stimulation. See, for example, at least U.S. Application Ser. No. 12/099,759, entitled METHODS OF PRODUCING PANCREATIC HORMONES, filed Apr. 8, 2008; Ser. No. 12/618,659, entitled Encapsulation of pancreatic lineage cells derived from human pluripotent stem cells, filed Nov. 13, 2009; Ser. No. 14/106,330, entitled IN VITRO DIFFERENTIATION OF PLURIPOTENT STEM CELLS TO PANCREATIC ENDODERM CELLS (PEC) AND IMMATURE BETA CELLS, filed Dec. 12, 2013; Ser. No. 14/201,630, filed Mar. 7, 2014; and PCT/US2014/026529, IN VITRO DIFFERENTIATION OF PLURIPOTENT STEM CELLS TO PANCREATIC ENDODERM CELLS (PEC) AND ENDOCRINE CELLS, filed Mar. 13, 2014; PCT/US2014/034425, TOOLS AND INSTRUMENTS FOR USE WITH IMPLANTABLE ENCSAPSULATION DEVICES, filed Apr. 16, 2014; U.S. application Ser. No. 14/254,844, TOOLS AND INSTRUMENTS FOR USE WITH IMPLANTABLE ENCSAPSULATION DEVICES, filed Apr. 16, 2014; and 29/488,209 CASE FOR AN ENCAPSULATION DEVICE, filed Apr. 16, 2014 and U.S. Design application No. 29/488,204, DEPLOYMENT TOOL FOR AN ENCAPSULATION DEVICE, filed Apr. 16, 2014; U.S. Design application No. 29/488,191, SIZING TOOL FOR AN ENCAPSULATION DEVICE, filed Apr. 16, 2014; and U.S. Design application No. 29/488,217, FILL POUCH ASSEMBLY FOR ENCAPSULATION DEVICE, filed Apr. 16, 2014.

### Further Examples

The examples disclosed herein include the following.

For Examples 1 to 110 below, a non-NWF layer is described as a “membrane,” however, any other non-NWF can be used in place of a “membrane”, as defined herein.

Example 1: A multi-layered cell encapsulation device comprising: a non-woven fabric (NWF) layer laser tack welded to a membrane, wherein the membrane defines a chamber of the device that is configured to receive cells therein; and a first film layer located on a first side of the NWF layer laser tack welded to the membrane, and a second film layer located on the opposite side of the NWF layer laser tack welded to the membrane; wherein the non-woven fabric (NWF) layer laser tack welded to the membrane comprises a device perimeter, flow holes spaced apart along a portion of the device perimeter, and an internal slit along a central longitudinal axis of the non-woven fabric (NWF) layer laser tack welded to the membrane, and wherein the perimeter of the non-woven fabric (NWF) layer laser tack welded to the membrane is recessed toward the central longitudinal axis relative to the first and second film layers.

Example 2: The device of any example herein, particularly example 1, wherein the membrane is a semi-permeable membrane.

Example 3: The device of any example herein, particularly example 1, wherein the membrane is perforated in an area that defines the chamber and that is interior to the device perimeter.

Example 4: The device of any example herein, particularly example 1, wherein the membrane and the non-woven fabric layers are perforated in an area that defines the chamber and that is interior to the device perimeter.

Example 5: The device of any example herein, particularly example 1, wherein the membrane is made of polytetrafluoroethylene (PTFE) or expanded polytetrafluoroethylene (ePTFE).

Example 6: The device of any example herein, particularly example 1, wherein cells are loaded into the device.

Example 7: The device of any example herein, particularly example 6, wherein the cells are definitive endoderm-lineage cells, pancreatic and duodenal homeobox 1 (PDX1)-positive pancreatic endoderm cells, pancreatic progenitor cells, pancreatic endocrine cells, or pancreatic beta cells.

Example 8: A cell encapsulation device, comprising: a) a first non-woven fabric (NWF) layer welded to a first membrane, wherein the first NWF layer is external to the first membrane; b) a first film layer located on a first side of the first NWF layer welded to the first membrane, and a second film layer located on the other side of the first NWF layer welded to the first membrane, wherein the first film layer is external to the second film layer; c) a second non-woven fabric (NWF) layer welded to a second membrane, wherein the second NWF layer is external to the second membrane; and d) a third film layer located on a first side of the second NWF layer welded to the second membrane, and a fourth film layer located on the other side of the second NWF layer welded to the second membrane, wherein the third film layer is external to the fourth film layer; wherein the first non-woven fabric (NWF) layer welded to the first membrane comprises a device perimeter, flow holes along a portion of the device perimeter, and an internal slit along the longest axis of the first non-woven fabric (NWF) layer welded to the first membrane, and the first non-woven fabric (NWF) layer welded to the first membrane is recessed in relationship to the first and second film layers; wherein the second non-woven fabric (NWF) layer welded to a second membrane comprises a device perimeter, flow holes along a portion of the device perimeter, and an internal slit along the longest axis of the second non-woven fabric (NWF) layer welded to the second membrane, and the second non-woven fabric (NWF) layer welded to the second membrane is recessed in relationship to the third and fourth film layers; wherein the first and second membranes and the first, second, third, and fourth film layers form at least one cell chamber.

Example 9: The device of any example herein, particularly example 8, wherein the welding is laser tack welding.

Example 10: The device of any example herein, particularly example 8, wherein the first and second membranes are semi-permeable membranes.

Example 11: The device of any example herein, particularly example 8, wherein the first and second membranes are perforated.

Example 12: The device of any example herein, particularly example 8, wherein the first and second membranes and the first and second non-woven fabric layers are perforated.

Example 13: The device of any example herein, particularly example 8, wherein the first and second membranes are made of polytetrafluoroethylene (PTFE) or expanded polytetrafluoroethylene (ePTFE).

Example 14: The device of any example herein, particularly example 8, wherein cells are loaded into the device.

Example 15: The device of any example herein, particularly example 14, wherein the cells are definitive endoderm-lineage cells, pancreatic and duodenal homeobox 1 (PDX1)-positive pancreatic endoderm cells, pancreatic progenitor cells, pancreatic endocrine cells, or pancreatic beta cells.

Example 16: The device of any example herein, particularly example 8, wherein the first and second membranes and the first, second, third, and fourth film layers form two or more cell chambers.

Example 17: The device of any example herein, particularly example 8, further comprising at least one loading port.

Example 18: A method of producing insulin in a mammal, said method comprising: implanting the device of any example herein, particularly example 15 into a mammalian host and maturing the cells in vivo into insulin producing pancreatic beta cells, thereby producing insulin in the mammal.

Example 19: The method of any example herein, particularly example 18, wherein the cells are cell aggregates.

Example 20: A method of producing hormone secreting cells, said method comprising implanting the device of any example herein, particularly example 15 into a mammalian host and maturing the cells in vivo into hormone secreting cells.

Example 21: A cell encapsulation device for use in a method of producing insulin in a mammal, comprising: the device of example 15; and wherein the method comprises loading definitive endoderm-lineage cells, pancreatic and duodenal homeobox 1 (PDX1)-positive pancreatic endoderm cells, pancreatic progenitor cells, pancreatic endocrine cells, or pancreatic beta cells into the at least one cell chamber of the device; implanting the device into the mammal; and maturing the implanted cells in the mammal into insulin producing pancreatic beta cells, thereby producing insulin in the mammal.

Example 22: A cell encapsulation device comprising, in order: a) a first film layer; b) a first mesh layer; c) a second film layer; d) a first NWF layer laser tack welded to a first membrane; e) a third film layer; f) at least one device port; g) a fourth film layer; h) a second NWF layer laser tack welded to a second membrane; i) a fifth film layer; j) a second mesh layer; and k) a sixth film layer; wherein the first NWF layer is external to the first cell-excluding membrane and the second NWF is external to the second cell-excluding membrane, and wherein the first NWF layer laser tack welded to the first cell-excluding membrane is recessed from the second and third film layers and the second NWF layer laser tack welded to the second cell-excluding membrane is recessed from the fourth and fifth film layers, and wherein the first NWF layer laser tack welded to the first cell-excluding membrane comprises a device perimeter, flow holes along a portion of the device perimeter, and an internal slit along the longest axis of the first NWF layer laser tack welded to the first cell-excluding membrane, and the second NWF layer laser tack welded to the second cell-excluding membrane comprises a device perimeter, flow holes along a portion of the device perimeter, and an internal slit along the longest axis of the second NWF laser tack welded to the second cell-excluding membrane, and wherein the first and second membranes and the second, third, fourth, and fifth film layers form at least one cell chamber.

Example 23: A method of making a multilayer cell encapsulation device, comprising: placing a first membrane on top of a first NWF layer; using a laser, laser tack welding the first membrane to the first NWF layer while cutting a device perimeter, flow holes along a portion of the device perimeter, and an internal slit along the longest axis of the first membrane and the first NWF layer; placing a second membrane on top of a second NWF layer; using a laser, laser tack welding the second membrane to the second NWF layer while cutting a device perimeter, flow holes along a portion of the device perimeter, and an internal slit along the longest axis of the second membrane and the second NWF layer; using the laser, individually cutting a first, second, third, fourth, fifth and sixth layers of film and a first and second layer of mesh in the shape of the device; placing in a cavity, in the following order: first film layer, first mesh layer, second film layer, first membrane layer laser tacked welded to a first NWF layer wherein the NWF is placed face down; third film layer, at least one device port, fourth film layer, second membrane layer laser tack welded to a second NWF wherein the membrane is placed face down, a fifth film layer, a second mesh layer and a sixth film layer; and welding the layers together in the cavity.

Example 24: The method of any example herein, particularly example 23, wherein the welding of step g) is ultrasonic welding or heat staking.

Example 25: The method of any example herein, particularly example 23, further comprising, wherein the welded product is removed from the cavity, flipped over, placed back into the cavity and welded again.

Example 26: The method of any example herein, particularly example 25, wherein the welding is ultrasonic or heat staking.

Example 27: A method of making a multilayer cell encapsulation device, comprising: a) place a first membrane on top of a first NWF layer; b) using a laser, laser tack weld the first membrane to the first NWF layer while cutting a device perimeter, flow holes along a portion of the device perimeter, and an internal slit along the longest axis of the first membrane and the first NWF layer; c) place a second membrane on top of a second NWF layer; d) using a laser, laser tack weld the second membrane to the second NWF layer while cutting a device perimeter, flow holes along a portion of the device perimeter, and an internal slit along the longest axis of the second membrane and the second NWF layer; e) using the laser, individually cut a first, second, third, fourth, fifth and sixth layers of film and a first and second layer of mesh in the shape of the device; f) place in a first cavity, in the following order: first film layer, first mesh layer, second film layer, first membrane layer laser tacked welded to a first NWF layer wherein the NWF is placed face down; and third film layer; g) weld the layers of step f) together in the first cavity; h) place in a second cavity, in the following order fourth film layer, second mesh layer, fifth film layer, second membrane layer laser tack welded to a second NWF layer wherein the NWF layer is placed face down, and sixth film layer; i) weld the layers of step h) together in the second cavity; j) place in a third cavity, the welded products of steps g), a device port, and the welded product of step i), wherein the first NWF and the second NWF layers are external to the center of the device; and k) weld the layers of step j) together in the third cavity, wherein the first, second, and third cavities optionally can be the same cavity if the welded products of steps g) and i) are each removed prior to step j).

Example 28: The method of any example herein, particularly example 27, wherein the welding of step g), step i), and step k) is ultrasonic or heat staking.

Example 29: The method of any example herein, particularly example 27, further comprising an additional step 1) wherein the welded product of step k) is removed from the cavity, flipped over, placed back into the cavity and welded again.

Example 30: The method of any example herein, particularly example 29, wherein the welding of step g), step i), step k), and step 1) is ultrasonic welding or heat staking.

Example 31: A multi-layered cell encapsulation device, comprising: a non-non-woven fabric (NWF) layer defining at least one chamber configured to receive cells therein; and a non-woven fabric (NWF) layer arranged external to the non-NWF layer, wherein the NWF layer and the non-NWF layer are welded together, and wherein at least a portion of a perimeter of at least the non-NWF layer which forms an outer weld of the device comprises a plurality of spaced apart flow holes extending along the perimeter.

Example 32: The multi-layered cell encapsulation device of any example herein, particularly example 31, further comprising a first film layer and a second film layer, the first film layer arranged exterior to the NWF layer and the second film layer arranged internal to non-NWF layer, and wherein perimeters of the first and second film layers, the non-NWF layer, and the NWF layer are welded together to form the outer weld.

Example 33: The multi-layered cell encapsulation device of any example herein, particularly example 32, wherein the at least one chamber is further defined inside the outer weld.

Example 34: The multi-layer cell encapsulation device of any example herein, particularly either example 32 or example 33, wherein the perimeter of the non-NWF layer and the perimeter of the NWF layer are recessed relative to a perimeter of the first film ring and the second film ring.

Example 35: The multi-layered cell encapsulation device of any example herein, particularly any one of examples 31-34, wherein the non-NWF layer is a first non-NWF layer and the NWF layer is a first NWF layer, and further comprising a second non-NWF layer and a second NWF layer arranged external to the second non-NWF layer, wherein the second non-NWF layer and NWF layer are welded together, and wherein the at least one chamber is defined between the first non-NWF layer and second non-NWF layer and inside the outer weld.

Example 36: The multi-layered cell encapsulation device of any example herein, particularly any one of examples 31-35, wherein the non-NWF layer comprises a plurality of spaced apart perforations in an area of the non-NWF layer that defines the at least one chamber and is internal to the outer weld.

Example 37: The multi-layered cell encapsulation device of any example herein, particularly any one of examples 31-36, wherein the NWF layer and the non-NWF layer comprise a plurality of spaced apart perforations in an area that defines the at least one chamber and is internal to the outer weld.

Example 38: The multi-layer cell encapsulation device of any example herein, particularly any one of examples 31-37, wherein the NWF layer and non-NWF layer are laser tack welded together.

Example 39: The multi-layer cell encapsulation device of any example herein, particularly any one of examples 31-38, wherein the flow holes have a length or a diameter in a range of 0.001 mm to 20 mm.

Example 40: The multi-layer cell encapsulation device of any example herein, particularly any one of examples 31-39, wherein the perimeter of the non-NWF layer is defined by opposing long edges that are connected together by two opposing non-straight edges, and wherein the flow holes are spaced apart from one another along the long edges.

Example 41: The multi-layer cell encapsulation device of any example herein, particularly example 40, wherein the plurality of flow holes are arranged in a linear array of spaced apart flow holes in each of the long edges.

Example 42: The multi-layer cell encapsulation device of any example herein, particularly any one of examples 31-41, wherein the non-NWF layer is a semi-permeable membrane.

Example 43: The multi-layer cell encapsulation device of any example herein, particularly example 42, wherein the semi-permeable membrane is made of polytetrafluoroethylene (PTFE) or expanded polytetrafluoroethylene (ePTFE).

Example 44: The multi-layer cell encapsulation device of any example herein, particularly any one of examples 31-41, wherein the non-NWF layer is a cell-excluding membrane.

Example 45: The multi-layer cell encapsulation device of any example herein, particularly any one of examples 31-41, wherein the non-NWF layer is a vascularizing membrane.

Example 46: The multi-layer cell encapsulation device of any example herein, particularly any one of examples 31-41, wherein the non-NWF layer is a mesh.

Example 47: The multi-layer cell encapsulation device of any example herein, particularly any one of examples 31-46, further comprising cells loaded inside the at least one chamber.

Example 48: The multi-layer cell encapsulation device of any example herein, particularly example 47, wherein the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells.

Example 49: The multi-layer cell encapsulation device of any example herein, particularly example 48, wherein the cells are aggregates.

Example 50: A multi-layered cell encapsulation device, comprising: a NWF layer and a non-NWF layer that are welded together, wherein the NWF layer and non-NWF layer comprise a plurality of spaced apart flow holes that extend along longitudinally extending edges of a perimeter of the welded NWF layer and non-NWF layer; a first film layer welded to the NWF layer on a first side of the welded NWF layer and non-NWF layer; a second film layer welded to the non-NWF layer on an opposite, second side of the welded NWF layer and non-NWF layer, wherein the welded together first film layer, second film layer, NWF layer, and non-NWF layer form an outer weld around a perimeter of the device, and wherein the plurality of spaced apart flow holes are disposed within the outer weld; and at least one chamber defined by the non-NWF layer, on the second side of the welded NWF layer and non-NWF layer, and inside the outer weld.

Example 51: A multi-layered cell encapsulation device, comprising: a non-NWF layer defining at least one chamber configured to receive cells therein; a NWF layer arranged external to the non-NWF layer, wherein the NWF layer and the non-NWF layer are welded together and are placed between and welded to a first film layer and a second film layer, wherein a perimeter of the NWF layer and non-NWF layer is recessed relative to a perimeter of the first and second film layers, and wherein the perimeter of the NWF layer and non-NWF and the perimeter of the first and second film layers form an outer weld of the device.

Example 52: The multi-layered cell encapsulation device of any example herein, particularly example 51, wherein the at least one chamber is further defined inside the outer weld.

Example 53: The multi-layered cell encapsulation device of any example herein, particularly either example 51 or example 52, wherein the non-NWF layer is a first non-NWF layer and the NWF layer is a first NWF layer, and further comprising a second non-NWF layer and a second NWF layer arranged external to the second non-NWF layer, wherein the second non-NWF layer and NWF layer are welded together, and wherein the at least one chamber is defined between the first non-NWF layer and second non-NWF layer and inside the outer weld.

Example 54: The multi-layered cell encapsulation device of any example herein, particularly any one of examples 51-53, wherein the perimeter of the NWF layer and the non-NWF layer comprise a plurality of spaced apart flow holes.

Example 55: The multi-layered cell encapsulation device of any example herein, particularly any one of examples 51-54, wherein the non-NWF layer comprises a plurality of spaced apart perforations in an area of the non-NWF layer that defines the at least one chamber and is internal to the outer weld.

Example 56: The multi-layered cell encapsulation device of any example herein, particularly any one of example 51-55, wherein the NWF layer and the non-NWF layer comprise a plurality of spaced apart perforations in an area that defines the at least one chamber and is internal to the outer weld.

Example 57: The multi-layered cell encapsulation device of any example herein, particularly any one of examples 51-56, wherein the NWF layer and non-NWF layer are laser tack welded together.

Example 58: The multi-layered cell encapsulation device of any example herein, particularly any one of examples 51-57, wherein the perimeter of the NWF layer and non-NWF layer is recessed from the perimeter of the first and second film layers by 1.0 mm+/−0.5 mm around the entire perimeter of the device.

Example 59: The multi-layer cell encapsulation device of any example herein, particularly any one of examples 51-58, wherein the non-NWF layer is a semi-permeable membrane.

Example 60: The multi-layer cell encapsulation device of any example herein, particularly any one of examples 51-58, wherein the non-NWF layer is a cell-excluding membrane.

Example 61: The multi-layer cell encapsulation device of any example herein, particularly any one of examples 51-58, wherein the non-NWF layer is a vascularizing membrane.

Example 62: The multi-layer cell encapsulation device of any example herein, particularly any one of examples 51-61, wherein the non-NWF layer is a mesh.

Example 63: The multi-layer cell encapsulation device of any example herein, particularly any one of examples 51-62, further comprising cells loaded inside the at least one chamber.

Example 64: The multi-layer cell encapsulation device of any example herein, particularly example 63, wherein the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells.

Example 65: A multi-layered cell encapsulation device, comprising: pancreatic lineage cells and a NWF layer and a non-NWF layer, wherein the NWF layer and the non-NWF layer are suction welded together and comprise device perimeters comprising flow-holes.

Example 66: A multi-layered cell encapsulation device, comprising: a non-woven fabric (NWF)/non-NWF layer comprising a NWF welded to an exterior of a non-NWF layer; a first film layer welded to a NWF side of the NWF/non-NWF layer; a second film layer welded to a non-NWF side of the NWF/non-NWF layer, wherein the welded together NWF/non-NWF layer, first film layer, and second film layer form an outer weld around the perimeter of the device and a longitudinally extending internal weld, and wherein in an area of the outer weld and/or internal weld, the NWF/non-NWF layer comprises one or more flow features that create one or more gaps in the NWF/non-NWF layer for the first film layer and the second film layer to bond together therethrough; and a first chamber defined by the non-NWF side of the NWF/non-NWF layer and between the outer weld and a first side of the internal weld; and a second chamber defined by the non-NWF side of the NWF/non-NWF layer and between the outer weld and a second side of the internal weld, wherein the first and second chambers are configured to receive cells therein.

Example 67: The multi-layered cell encapsulation device of any example herein, particularly example 66, wherein the one or more flow features includes a plurality of spaced apart flow holes extending along at least a portion of a perimeter of the NWF/non-NWF layer in an area of the outer weld.

Example 68: The multi-layered cell encapsulation device of any example herein, particularly either example 66 or example 67, wherein the one or more flow features includes an end cap formed by a perimeter of the NWF/non-NWF layer being offset, inward toward a center of the device, from a perimeter of the first and second film layers.

Example 69: The multi-layered cell encapsulation device of any example herein, particularly example 68, wherein the perimeter of the NWF/non-NWF layer is offset from the perimeter of the first and second film layers by 1.0 mm+/−0.5 mm around the entire perimeter of the device.

Example 70: The multi-layered cell encapsulation device of any example herein, particularly any one of examples 66-69, wherein the one or more flow features includes a longitudinally extending internal slit in an area of the internal weld.

Example 71: The multi-layered cell encapsulation device of any example herein, particularly example 70, wherein the longitudinally extending slit extends along a majority portion of the internal weld.

Example 72: The multi-layered cell encapsulation device of any example herein, particularly any one of examples 66-71, further comprising a first port extending into an interior of the first chamber for loading cells into the first chamber and a second port extending into an interior of the second chamber for loading cells into the second chamber.

Example 73: The multi-layered cell encapsulation device of any example herein, particularly any one of examples 66-72, wherein the NWF/non-NWF layer comprises a plurality of spaced apart perforations in an area that defines the first chamber and in an area that defines the second chamber.

Example 74: The multi-layered cell encapsulation device of any example herein, particularly any one of examples 66-73, wherein the NWF and non-NWF layer of the NWF/non-NWF layer are laser tack welded together.

Example 75: The multi-layer cell encapsulation device of any example herein, particularly any one of examples 66-74, wherein the non-NWF layer is a semi-permeable membrane.

Example 76: The multi-layer cell encapsulation device of any example herein, particularly any one of examples 66-74, wherein the non-NWF layer is a cell-excluding membrane.

Example 77: The multi-layer cell encapsulation device of any example herein, particularly any one of examples 66-74, wherein the non-NWF layer is a vascularizing membrane.

Example 78: The multi-layer cell encapsulation device of any example herein, particularly any one of examples 66-74, wherein the non-NWF layer is a mesh.

Example 79: The multi-layer cell encapsulation device of any example herein, particularly any one of examples 66-78, further comprising cells loaded inside the first chamber and the second chamber.

Example 80: The multi-layer cell encapsulation device of any example herein, particularly example 79, wherein the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells.

Example 81: A method for assembling a multi-layered cell encapsulation device, comprising: suction welding a NWF layer and a non-NWF layer together to form a NWF/non-NWF layer, the suction welding including cutting a perimeter of the NWF/non-NWF layer of the NWF/non-NWF layer to be recessed relative to remaining layers of the device when arranged together with the remaining layers; and welding the remaining layers of the device together with the NWF/non-NWF layer, the remaining layers including a first film layer arranged on an exterior, NWF side of the NWF/non-NWF layer and a second film layer arranged on an interior, non-NWF side of the NWF/non-NWF layer, wherein the welded together device comprises an outer weld around a perimeter of the device and at least one chamber defined inside the outer weld and by the non-NWF side of the NWF/non-NWF layer.

Example 82: The method of any example herein, particularly example 81, wherein the suction welding and cutting is laser tack welding.

Example 83: The method of any example herein, particularly either example 81 or example 82, wherein welding the remaining layers of the device together with the NWF/non-NWF layer includes welding via ultrasonic welding or heat staking.

Example 84: The method of any example herein, particularly any one of examples 81-83, wherein welding the remaining layers of the device together with the NWF/non-NWF layer includes melting and bonding together the first film layer and the second film layer across a gap formed in the NWF/non-NWF layer by the recessed perimeter of the NWF/non-NWF layer relative to the perimeter of the first and second film rings.

Example 85: The method of any example herein, particularly any one of examples 81-84, wherein the suction welding further includes cutting flow holes in the perimeter of the NWF/non-NWF layer, wherein the flow holes are spaced apart from one another along one or more edges defining the perimeter.

Example 86: The method of any example herein, particularly example 85, wherein welding the remaining layers of the device together with the NWF/non-NWF layer includes melting and bonding together the first film layer and the second film layer through the flow holes.

Example 87: The method of any example herein, particularly any one of examples 81-86, wherein the at least one chamber of the device includes a first chamber and a second chamber, wherein the welded together device comprises an interior weld separating the first chamber and the second chamber, and wherein the suction welding further includes cutting a longitudinally extending internal slit in the NWF/non-NWF layer in an area of the internal weld.

Example 88: The method of any example herein, particularly example 87, wherein welding the remaining layers of the device together with the NWF/non-NWF layer includes melting and bonding together the first film layer and the second film layer through the internal slit.

Example 89: The method of any example herein, particularly any one of examples 81-88, wherein the non-NWF layer is a semi-permeable membrane.

Example 90: The method of any example herein, particularly example 89, wherein the semi-permeable membrane is made of polytetrafluoroethylene (PTFE) or expanded polytetrafluoroethylene (ePTFE).

Example 91: The method of any example herein, particularly any one of examples 81-88, wherein the non-NWF layer is a cell-excluding membrane.

Example 92: The method of any example herein, particularly any one of examples 81-88, wherein the non-NWF layer is vascularizing membrane.

Example 93: The method of any example herein, particularly any one of examples 81-88, wherein the non-NWF layer is a mesh.

Example 94: The method of any example herein, particularly any one of examples 81-93, wherein the non-NWF layer is perforated.

Example 95: The method of any example herein, particularly any one of examples 81-94, wherein the NWF layer and the non-NWF layer are perforated.

Example 96: A multi-layered cell encapsulation device, comprising: a first non-woven fabric (NWF) layer welded to a first non-NWF layer, wherein the first NWF layer is external to the first non-NWF layer; a first film layer arranged external to the first NWF layer welded to the first non-NWF layer; a second film layer arranged internal to the first non-NWF layer of the welded together first NWF layer and first non-NWF layer; a second NWF layer welded to a second non-NWF layer, wherein the second NWF layer is external to the second non-NWF layer; a third film layer arranged external to the second NWF layer welded to the second non-NWF layer; a fourth film layer arranged internal to the second non-NWF layer of the welded together second NWF layer and second non-NWF layer; and at least one chamber configured to receive cells therein, the at least one chamber defined between the first non-NWF layer and the second non-NWF layer and by the second film layer and fourth film layer; wherein perimeters of the first NWF layer welded to the first non-NWF layer, the second NWF layer welded to the second non-NWF layer, and the first, second, third, and fourth film layers are welded together to form an outer weld of the device; and wherein at least a portion of the perimeters of the first NWF layer welded to the first non-NWF layer and the second NWF layer welded to the second non-NWF layer comprise a plurality of spaced apart flow holes extending along the at least the portion of the perimeters.

Example 97: The multi-layered cell encapsulation device of any example herein, particularly example 96, wherein the perimeter of the first NWF layer welded to the first non-NWF layer and the perimeter of the second NWF layer welded to the second non-NWF layer are recessed relative to the perimeters of the first, second, third, and fourth film rings such that end caps are formed in the first and second NWF layers welded to the first and second non-NWF layers, respectively.

Example 98: The multi-layered cell encapsulation device of any example herein, particularly either example 96 or example 97, wherein the first and second non-NWF layers comprise a plurality of spaced apart perforations in an area of the first and second non-NWF layers that define the at least one chamber and is internal to the outer weld.

Example 99: The multi-layer cell encapsulation device of any example herein, particularly any one of examples 96-98, wherein the first NWF layer and first non-NWF layer are laser tack welded together, and wherein the second NWF layer and second non-NWF layer are laser tack welded together.

Example 100: The multi-layer cell encapsulation device of any example herein, particularly any one of examples 96-99, wherein the flow holes have a length or a diameter in a range of 0.001 mm to 20 mm.

Example 101: The multi-layer cell encapsulation device of any example herein, particularly any one of examples 96-100, wherein the perimeter of the first NWF layer welded to the first non-NWF layer and the perimeter of the second NWF layer welded to the second non-NWF layer are each defined by opposing long edges that are connected together by two opposing non-straight edges, and wherein the flow holes are spaced apart from one another along the long edges.

Example 102: The multi-layer cell encapsulation device of any example herein, particularly example 101, wherein the plurality of flow holes are arranged in a linear array of spaced apart flow holes in the long edges.

Example 103: The multi-layer cell encapsulation device of any example herein, particularly any one of examples 96-102, wherein the non-NWF layer is a semi-permeable membrane.

Example 104: The multi-layer cell encapsulation device of any example herein, particularly example 103, wherein the semi-permeable membrane is made of polytetrafluoroethylene (PTFE) or expanded polytetrafluoroethylene (ePTFE).

Example 105: The multi-layer cell encapsulation device of any example herein, particularly any one of examples 96-102, wherein the non-NWF layer is a cell-excluding membrane.

Example 106: The multi-layer cell encapsulation device of any example herein, particularly any one of examples 96-102, wherein the non-NWF layer is a vascularizing membrane.

Example 107: The multi-layer cell encapsulation device of any example herein, particularly any one of examples 96-102, wherein the non-NWF layer is a mesh.

Example 108: The multi-layer cell encapsulation device of any example herein, particularly any one of examples 96-107, further comprising cells loaded inside the at least one chamber.

Example 109: The multi-layer cell encapsulation device of any example herein, particularly example 108, wherein the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells.

Example 110: The multi-layer cell encapsulation device of any example herein, particularly example 109, wherein the cells are aggregates.

The terms and expressions employed herein are used as terms and expressions of description and not of limitation, and there is no intention, in the use of such terms and expressions, of excluding any equivalents of the features shown and described or portions thereof. In addition, having described certain examples of the disclosure, it will be apparent to those of ordinary skill in the art that other examples incorporating the concepts disclosed herein may be used without departing from the spirit and scope of the disclosure. In particular, examples of the disclosure need not include all of the features nor have all of the advantages described herein. Rather, they may possess any subset or combination of features and advantages. Accordingly, the described examples are to be considered in all respects as only illustrative and not restrictive.

While certain examples have been shown and described herein, it will be obvious to those skilled in the art that such examples are provided by way of example only. Numerous variations, changes, and substitutions will now occur to those skilled in the art without departing from the disclosure. It should be understood that various alternatives to the examples of the disclosure described herein might be employed in practicing the disclosure. It is intended that the following claims define the scope of the disclosure and that methods and structures within the scope of these claims and their equivalents be covered thereby.

1. A multi-layered cell encapsulation device, comprising:
a non-non-woven fabric (non-NWF) layer defining at least one chamber configured to receive cells therein; and
a non-woven fabric (NWF) layer arranged external to the non-NWF layer, wherein the NWF layer and the non-NWF layer are welded together, and wherein at least a portion of a perimeter of at least the non-NWF layer which forms an outer weld of the device comprises a plurality of spaced apart flow holes extending along the perimeter.

2. The multi-layered cell encapsulation device of claim 1, further comprising a first film layer and a second film layer, the first film layer arranged exterior to the NWF layer, and the second film layer arranged internal to non-NWF layer, and wherein perimeters of the first and second film layers, the non-NWF layer, and the NWF layer are welded together to form the outer weld.

3. The multi-layered cell encapsulation device of claim 2, wherein the at least one chamber is further defined inside the outer weld.

4. The multi-layer cell encapsulation device of claim 2, wherein the perimeter of the non-NWF layer and the perimeter of the NWF layer are recessed relative to a perimeter of the first film ring and the second film ring.

5. The multi-layered cell encapsulation device of claim 1, wherein the non-NWF layer is a first non-NWF layer and the NWF layer is a first NWF layer, and further comprising a second non-NWF layer and a second NWF layer arranged external to the second non-NWF layer, wherein the second non-NWF layer and second NWF layer are welded together, and wherein the at least one chamber is defined between the first non-NWF layer and second non-NWF layer and inside the outer weld.

6. The multi-layered cell encapsulation device of claim 1, wherein the non-NWF layer comprises a plurality of spaced apart perforations in an area of the non-NWF layer that defines the at least one chamber and is internal to the outer weld.

7. The multi-layer cell encapsulation device of claim 1, wherein the NWF layer and non-NWF layer are laser tack welded together.

8. The multi-layer cell encapsulation device of claim 1, wherein the perimeter of the non-NWF layer is defined by opposing long edges that are connected together by two opposing non-straight edges, and wherein the flow holes are spaced apart from one another along the long edges.

9. The multi-layer cell encapsulation device of claim 8, wherein the flow holes of the plurality of flow holes are arranged in a linear array of spaced apart flow holes in each of the long edges.

10. The multi-layer cell encapsulation device of claim 1, wherein the non-NWF layer is a cell-excluding membrane.

11. The multi-layer cell encapsulation device of claim 1, further comprising cells loaded inside the at least one chamber.

12. The multi-layer cell encapsulation device of claim 11, wherein the cells are definitive endoderm-lineage cells, pancreatic lineage cells, pancreatic and duodenal homeobox 1 (PDX1)-positive pancreatic endoderm cells, pancreatic progenitor cells, pancreatic endocrine cells, or pancreatic beta cells.

13. A multi-layered cell encapsulation device, comprising:
a non-woven fabric (NWF)/non-NWF layer comprising a NWF welded to an exterior of a non-NWF layer;
a first film layer welded to a NWF side of the NWF/non-NWF layer;
a second film layer welded to a non-NWF side of the NWF/non-NWF layer, wherein the welded together NWF/non-NWF layer, first film layer, and second film layer form an outer weld around a perimeter of the device and a longitudinally extending internal weld, and wherein in an area of the outer weld and/or internal weld, the NWF/non-NWF layer comprises one or more flow features that create one or more gaps in the NWF/non-NWF layer for the first film layer and the second film layer to bond together therethrough;
a first chamber defined by the non-NWF side of the NWF/non-NWF layer and between the outer weld and a first side of the internal weld; and
a second chamber defined by the non-NWF side of the NWF/non-NWF layer and between the outer weld and a second side of the internal weld, wherein the first and second chambers are configured to receive cells therein.

14. The multi-layered cell encapsulation device of claim 13, wherein the one or more flow features includes a plurality of spaced apart flow holes extending along at least a portion of a perimeter of the NWF/non-NWF layer in an area of the outer weld.

15. The multi-layered cell encapsulation device of claim 13, wherein the one or more flow features includes a perimeter of the NWF/non-NWF layer being offset, inward toward a center of the device, from a perimeter of the first and second film layers.

16. The multi-layered cell encapsulation device of claim 13, wherein the one or more flow features includes a longitudinally extending internal slit in an area of the internal weld.

17. The multi-layered cell encapsulation device of claim 13, further comprising a first port extending into an interior of the first chamber for loading cells into the first chamber and a second port extending into an interior of the second chamber for loading cells into the second chamber.

18. The multi-layered cell encapsulation device of claim 13, wherein the NWF/non-NWF layer comprises a plurality of spaced apart perforations in an area that defines the first chamber and in an area that defines the second chamber.

19. (canceled)

20. A method for assembling a multi-layered cell encapsulation device, comprising:
suction welding a NWF layer and a non-NWF layer together to form a composite NWF/non-NWF layer, the suction welding comprising cutting a perimeter of the NWF/non-NWF layer to be recessed relative to remaining layers of the device when arranged together with the remaining layers; and
welding the remaining layers of the device together with the NWF/non-NWF layer, the remaining layers including a first film layer arranged on an exterior, NWF side of the NWF/non-NWF layer and a second film layer arranged on an interior, non-NWF side of the NWF/non-NWF layer, wherein the welded together device comprises an outer weld around a perimeter of the device and at least one chamber defined inside the outer weld and by the non-NWF side of the NWF/non-NWF layer.

21. The method of claim 20, wherein the suction welding and cutting is laser tack welding.

22-25. (canceled)
